(19) United States
(12) Patent Application Publication
(10) Pub. No.: US 2011/0097261 A1
(43) Pub. Date: Apr. 28, 2011

(54) AMIGO-2-INHIBITORS FOR TREATING, DIAGNOSING OR DETECTING CANCER

(76) Inventor: Mary J. Janatpour, Castro Valley, CA (US)

(21) Appl. No.: 12/309,436

(22) PCT Filed: Jul. 19, 2007
(86) PCT No.: PCT/US2007/073895

§ 371 Date: Jul. 2, 2009

Related U.S. Application Data
(60) Provisional application No. 60/807,885, filed on Jul. 20, 2006.
(60) Provisional application No. 60/870,025, filed on Dec. 14, 2006.

(51) Int. Cl. A61K 51/10; A61K 39/395; A61K 31/7088; A61K 38/00; G01N 33/53; C12N 5/09; A61K 49/00; A61K 51/04; A61K 51/08; A61K 51/06; C07K 16/18; C12N 5/16; A01K 67/00; C07K 2/00; C07H 21/02; A61K 31/7105; C07H 21/00; A61P 35/00

(52) U.S. Cl. 424/1.49; 424/139.1; 514/44 R; 514/19.3; 424/133.1; 424/135.1; 436/94; 436/86; 435/325; 424/9.1; 424/1.65; 424/1.85; 424/1.89; 424/1.69; 435/7.2; 530/387.9; 435/326; 800/13; 530/300; 536/23.1

(57) ABSTRACT

The invention provides, inter alia, methods for treating cancer, compositions for treating cancer, and methods and compositions for diagnosing and/or detecting cancer. In particular, the present invention provides compositions and methods for treating, diagnosing and detecting cancers associated with AMIGO-2 overexpression.

### FIELD OF THE INVENTION

The present invention relates generally to the field of oncology. More particularly, the invention relates to methods for treating cancer, compositions for treating cancer, and methods and compositions for diagnosing and/or detecting cancer.

### BACKGROUND OF THE INVENTION

Cancer is the second leading cause of death in the United States. Although “cancer” is used to describe many different types of cancer, i.e. breast, prostate, lung, colon, pancreas, each type of cancer differs both at the phenotypic level and the genetic level. The unregulated growth characteristic of cancer occurs when the expression of one or more genes becomes dysregulated due to mutations, and cell growth can no longer be controlled.

Genes are often classified in two classes, oncogenes and tumor suppressor genes. Oncogenes are genes whose normal function is to promote cell growth, but only under specific conditions. When an oncogene gains a mutation and then loses that control, it promotes growth under all conditions. However, it has been found that for cancer to be truly successful the cancer must also acquire mutations in tumor suppressor genes. The normal function of tumor suppressor genes is to stop cellular growth. Examples of tumor suppressors include p53, p16, p21, and APC, all of which, when acting normally, stop a cell from dividing and growing uncontrollably. When a tumor suppressor is mutated or lost, that brake on cellular growth is also lost, allowing cells to now grow without restraints.

AMIGO-2 (also known as Alivin 1 and DEGA) is a member of the AMIGO (amphoterin-induced gene and ORF) family (Kuja-Panula et al., JCB 160:963, 2003). The AMIGO family is involved in cell motility and family members have both homophilic and heterophilic interactions. AMIGO-2 also inhibits apoptosis and promotes survival of neurons (Ono et al., J Neurosci 23:5887, 2003). AMIGO-2 is up-regulated in gastric cancer (Rabenau et al., Oncogene 23:5056, 2004), and stable inhibition of AMIGO-2 can inhibit tumor cell chromosomal stability, migration and growth.

To date, however, the role of AMIGO-2 in cancer and other diseases and disorders has not been fully elucidated. Accordingly there is a need to identify compositions and methods that modulate AMIGO-2. The present invention is directed to these, as well as other, important needs.

### SUMMARY OF THE INVENTION

In some aspects, the present invention provides compositions comprising an AMIGO-2 modulator and one or more pharmaceutically acceptable carriers.

In one aspect, the invention features a composition comprising an AMIGO-2 inhibitor and one or more pharmaceutically acceptable carriers, wherein the AMIGO-2 inhibitor is an isolated double-stranded RNA (dsRNA); an isolated oligonucleotide comprising at least 10 consecutive nucleotides of a sequence selected from the group consisting of SEQ ID NOs:7-16; an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain; a small molecule; a mimetic; a soluble receptor; or a decoy.

In another aspect, the invention features a purified antibody that specifically binds to an epitope of an AMIGO-2 polypeptide, wherein the epitope is in the signal peptide domain, the LRRNT domain, LRR1 domain, LRR2 domain, LRR3 domain, LRR4 domain, LRR5 domain, LRR6 domain, LRRCT domain, Ig V-set domain, or Ig domain.

In yet another aspect, the invention features an isolated cell that produces an antibody that specifically binds to an epitope of an AMIGO-2 polypeptide, wherein the epitope is in the signal peptide domain, the LRRNT domain, LRR1 domain, LRR2 domain, LRR3 domain, LRR4 domain, LRR5 domain, LRR6 domain, LRRCT domain, Ig V-set domain, or Ig domain.

In another aspect, the invention features a hybridoma that produces an antibody that specifically binds to an epitope of an AMIGO-2 polypeptide, wherein the epitope is in the signal peptide domain, the LRRNT domain, LRR1 domain, LRR2 domain, LRR3 domain, LRR4 domain, LRR5 domain, LRR6 domain, LRRCT domain, Ig V-set domain, or Ig domain.

In yet another aspect, the invention features a non-human transgenic animal that produces an antibody that specifically binds to an epitope of an AMIGO-2 polypeptide, wherein the epitope is in the signal peptide domain, the LRRNT domain, LRR1 domain, LRR2 domain, LRR3 domain, LRR4 domain, LRR5 domain, LRR6 domain, LRRCT domain, Ig V-set domain, or Ig domain.

In another aspect, the invention features an isolated epitope-bearing fragment of the polypeptide of SEQ ID NO:2, wherein the fragment comprises one or more epitopes selected from the group consisting of SEQ ID NOs:3-6 and 25-62.

In yet another aspect, the invention features a polynucleotide that encodes an isolated epitope-bearing fragment of the polypeptide of SEQ ID NO:2, wherein the fragment comprises one or more epitopes selected from the group consisting of SEQ ID NOs:3-6 and 25-62.

In yet another aspect, the invention features a purified AMIGO-2 antibody which is obtained by immunization of a subject with the epitope-bearing fragment of the polypeptide of SEQ ID NO:2, wherein the fragment comprises one or more epitopes selected from the group consisting of SEQ ID NOs:3-6 and 25-62.

In another aspect, the invention features an isolated dsRNA molecule comprising a first strand of nucleotides comprising at least 19 consecutive nucleotides of a sequence set forth in SEQ ID NOs:17-24, and a second strand of nucleotides comprising a sequence substantially complementary to the first strand, wherein the dsRNA molecule is less than 3769 nucleotides long.

In yet another aspect, the invention features an isolated dsRNA molecule comprising a first strand of nucleotides comprising at least 19 consecutive nucleotides of a sequence set forth in SEQ ID NOs:17-24, and a second strand of nucleotides comprising a sequence fully complementary to the first strand, wherein the dsRNA molecule is less than 3769 nucleotides long.

In another aspect, the invention features an isolated nucleic acid comprising at least 10 consecutive nucleotides of a sequence set forth in SEQ ID NOs:7-16.

In another aspect, the invention features a method of treating cancer or a cancer symptom in a patient in need thereof comprising administering to the patient a therapeutically effective amount of an AMIGO-2 inhibitor, where the inhibitor is an isolated double-stranded RNA (dsRNA); an isolated oligonucleotide comprising at least 10 consecutive nucleotides of a sequence selected from the group consisting of SEQ ID NOs:7-16; an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain; a small molecule; a mimetic; a soluble receptor; or a decoy.

In yet another aspect, the invention features a method of modulating an AMIGO-2 activity in a patient, the method comprising administering to the patient an amount of the AMIGO-2 inhibitor effective to modulate the AMIGO-2 activity.

In yet another aspect, the invention features a method of identifying a patient susceptible to AMIGO-2 therapy comprising: (a) detecting the presence or absence of evidence of AMIGO-2 expression in the sample, wherein the presence of evidence of AMIGO-2 expression in the sample is indicative of a patient who is a candidate for AMIGO-2 therapy and the absence of evidence of AMIGO-2 expression in the sample is indicative of a patient who is not a candidate for AMIGO-2 therapy; (b) administering a therapeutically effective amount of a composition comprising an AMIGO-2 inhibitor to the patient if the patient is a candidate for AMIGO-2 therapy; and (c) administering a traditional cancer therapeutic to the patient if the patient is not a candidate for AMIGO-2 therapy.

In another aspect, the invention features a method of inhibiting growth of cancer cells comprising contacting the cancer cells with an amount of an AMIGO-2 inhibitor effective to inhibit growth of the cells by at least 20% as compared to a control.

In another aspect, the invention features a method of inhibiting a cancer cell phenotype in a patient in need thereof, the method comprising administering to the patient a therapeutically effective amount of an AMIGO-2 inhibitor.

In another aspect, the invention features a method of inhibiting cancer cell growth, the method comprising administering to a patient having a cancer comprising one or more cells expressing AMIGO-2 a compound that modulates of one or more downstream markers of AMIGO-2. The one or more downstream markers of AMIGO-2 can be c-MYC, c-Jun, FosL1, and Extracellular signal-Regulated Kinase (ERK). In some embodiments, modulation of the one or more downstream markers can be inhibition of expression of the one or more downstream markers, e.g., inhibition of protein or mRNA expression. In some embodiments, modulation can also include inhibition of the activity of the one or more downstream markers of AMIGO-2. In some embodiments, modulation of ERK includes modulation of the phosphorylation of ERK. In some embodiments, modulation of ERK includes modulation of ERK kinase activity towards one or more of ERK substrates.

In yet another aspect, the invention features a method for detecting a tumor in a patient comprising administering to the patient a composition comprising an AMIGO-2 inhibitor linked to an imaging agent and detecting the localization of the imaging agent in the patient.

In another aspect, the invention features a method for inhibiting the interaction of two or more cells in a patient comprising administering a therapeutically effective amount of an AMIGO-2 inhibitor to the patient.

In another aspect, the invention features a method of expressing an AMIGO-2 antibody in a cell wherein the AMIGO-2 antibody specifically binds to an epitope comprising a sequence selected from the group consisting of SEQ ID NOs:3-6 and 25-62. The method includes expressing a nucleic acid encoding the AMIGO-2 antibody in the cell.

In another aspect, the invention features a method of identifying a cancer inhibitor, where the cancer is characterized by overexpression of AMIGO-2 compared to a control. The method includes contacting a cell expressing AMIGO-2 with a candidate compound and determining whether an AMIGO-2 activity is modulated, wherein modulation of the AMIGO-2 activity is indicative of a cancer inhibitor.

In another aspect, the invention features a method of identifying a cancer inhibitor, where the cancer is characterized by overexpression of AMIGO-2 compared to a control. The method includes contacting a cell expressing AMIGO-2 with a candidate compound and an AMIGO-2 ligand, and determining whether an activity of a downstream marker of AMIGO-2 is modulated, wherein modulation of the downstream marker is indicative of a cancer inhibitor.

In another aspect, the invention features a method for determining the susceptibility of a patient to an AMIGO-2 inhibitor comprising detecting evidence of differential expression of AMIGO-2 in a cancer sample of the patient, wherein evidence of differential expression of AMIGO-2 is indicative of the patient's susceptibility to the AMIGO-2 inhibitor.

In another aspect, the invention features a method of purifying AMIGO-2 protein from a sample comprising: (a) providing an affinity matrix comprising an anti-AMIGO-2 antibody bound to a solid support; (b) contacting the sample with the affinity matrix to form an affinity matrix-AMIGO-2 protein complex; (c) separating the affinity matrix AMIGO-2 protein complex from the remainder of the sample; and (d) releasing AMIGO-2 protein from the affinity matrix.

In another aspect, the invention features a method of delivering a cytotoxic agent or a diagnostic agent to one or more cells that express AMIGO-2. The method includes: (a) providing the cytotoxic agent or the diagnostic agent conjugated to an anti-AMIGO-2 antibody or fragment thereof; and (b) exposing the cell to the antibody-agent or fragment-agent conjugate.

In another aspect, the invention features a method for determining the effectiveness of a candidate AMIGO-2 inhibitor comprising contacting AMIGO-2-expressing cells with the candidate AMIGO-2 inhibitor and determining whether a level or activity of a downstream AMIGO-2 marker is decreased, wherein a decrease in the level or activity of the downstream marker indicates that the candidate AMIGO-2 inhibitor is an effective anti-cancer medication.

In yet another aspect, the invention features a method for determining the effectiveness of a candidate AMIGO-2 inhibitor comprising contacting AMIGO-2-expressing cells with the candidate AMIGO-2 inhibitor and determining whether PARP1 cleavage is increased, wherein an increase in PARP1 cleavage indicates that the candidate AMIGO-2 inhibitor is an effective anti-cancer medication.

In yet another aspect, the invention features a method of determining whether a cancer is an AMIGO-2-related cancer comprising comparing AMIGO-2 expression in cancer and control cells, wherein upregulated AMIGO-2 expression in the cancer cells as compared to the control cells indicates that the cancer is an AMIGO-2 related cancer.

In another aspect, the invention features a method of determining whether a cancer is an AMIGO-2-related cancer comprising contacting a cancer sample and a control sample with an AMIGO-2 inhibitor, and comparing a level or activity of an AMIGO-2 downstream marker in the cancer sample and in the control sample, wherein decreased level or activity of the AMIGO-2 downstream marker in the cancer sample compared to the control sample indicates that the cancer is an AMIGO-2 related cancer.

In another aspect, the invention features a method of determining whether a cancer is an AMIGO-2-related cancer comprising contacting a cancer sample and a control sample with an AMIGO-2 inhibitor, and comparing PARP1 cleavage in the cancer sample and in the control sample, wherein increased PARP1 cleavage in the cancer sample compared to the control sample indicates that the cancer is an AMIGO-2 related cancer.

In yet another aspect, the invention features a method of treating a cancer patient comprising determining whether a cancer is an AMIGO-2-related cancer, and administering to the patient a composition comprising an AMIGO-2 inhibitor if the patient has an AMIGO-2-related cancer, and administering to the patient a traditional cancer therapeutic if the patient does not have an AMIGO-2-related cancer.

In another aspect, the invention features a method of treating a cancer patient comprising comparing AMIGO-2 expression in a cancer sample from the patient to AMIGO-2 expression in a control sample and (1) treating the patient with a composition comprising an AMIGO-2 inhibitor if AMIGO-2 expression is upregulated in the cancer sample as compared to the control sample; and (2) performing a secondary assay if AMIGO-2 expression is unchanged or downregulated in the cancer sample as compared to the control sample.

In another aspect, the invention features a method for diagnosing cancer in a patient comprising assaying for AMIGO-2 localization in candidate cancer cells, wherein when the ratio of AMIGO-2 localized to the cell membrane to AMIGO-2 localized to other areas of the cancer cells not including the cell membrane is at least 2:1, the patient is diagnosed as having an AMIGO-2-related cancer.

In another aspect, the invention features a method of detecting modulation of AMIGO-2 activity in a sample comprising cells which express AMIGO-2. The method includes: (a) contacting the sample with an AMIGO-2 inhibitor for a time sufficient to modulate AMIGO-2 activity; (b) immunoprecipitating AMIGO-2 with an anti-AMIGO-2 antibody; and (c) comparing AMIGO-2 serine/threonine phosphorylation in the sample to a control using a phospho-serine/threonine antibody. An alteration of serine/threonine phosphorylation of AMIGO-2 in the sample compared to the control is an indication of the modulation of AMIGO-2 activity.

In another aspect, the invention features a method of detecting modulation of AMIGO-2 activity in a sample comprising cells which express AMIGO-2. The method includes: (a) overexpressing AMIGO-2 in the sample for a time sufficient to modulate AMIGO-2 activity; (b) immunoprecipitating AMIGO-2 with an anti-AMIGO-2 antibody; and (c) comparing AMIGO-2 serine/threonine phosphorylation in the sample to a control using a phospho-serine/threonine antibody. An alteration of serine/threonine phosphorylation of AMIGO-2 in the sample compared to the control is an indication of the modulation of AMIGO-2 activity.

In yet another aspect, the invention features a method of detecting modulation of AMIGO-2 activity in a sample comprising cells which express AMIGO-2. The method includes: (a) contacting the sample with an AMIGO-2 inhibitor for a time sufficient to modulate AMIGO-2 activity; (b) immunoprecipitating AMIGO-2 with an anti-phospho-serine/threonine antibody, and (c) comparing the level of phosphorylated AMIGO-2 in the sample to a control using an anti-AMIGO-2 antibody. Alteration of the level of phosphorylated AMIGO-2 in the sample compared to the control is an indication of the modulation of AMIGO-2 activity.

In another aspect, the invention features a method of detecting modulation of AMIGO-2 activity in a sample comprising cells which express AMIGO-2. The method includes: (a) overexpressing AMIGO-2 in the sample for a time sufficient to modulate AMIGO-2 activity; (b) immunoprecipitating AMIGO-2 with an anti-phospho-serine/threonine antibody; and (c) comparing the level of phosphorylated AMIGO-2 in the sample to a control using an anti-AMIGO-2 antibody. Alteration of the level of phosphorylated AMIGO-2 in the sample compared to a control is an indication of the modulation of AMIGO-2 activity.

In another aspect, the invention features a method of identifying an AMIGO-2 modulator comprising comparing phosphorylation of AMIGO-2 in a sample comprising one or more cells expressing AMIGO-2 in the presence and absence of a candidate compound, wherein modulation of phosphorylation of AMIGO-2 in the sample in the presence of the candidate compound as compared to phosphorylation of AMIGO-2 in the sample in the absence of the candidate compound indicates that the candidate compound is an AMIGO-2 modulator.

These and other aspects of the present invention will be elucidated in the detailed description of the invention.

### BRIEF DESCRIPTION OF THE DRAWINGS

FIGS. 1A and 1B depict gene expression data generated from Affymetrix GeneChip® ((Human Genome U133 Plus 2.0 Array, Affymetrix, Inc.)) oligonucleotide arrays (FIG. 1A) and cDNA microarrays synthesized in-house (FIG. 1B).

FIG. 2 depicts a graphical representation of relative AMIGO-2 mRNA levels in normal tissues and in colon, breast, and prostate tissue samples.

FIG. 3 depicts a graphical representation of oligonucleotide array data (Human Genome U133 Plus 2.0 Array, Affymetrix, Inc.) from AMIGO-2 mRNA isolated from cancerous and normal tissues. Normal and cancerous tissue types are described along the x-axis. Each spot on the vertical axes represents a tissue sample from a single patient, and the height of each spot on the vertical axes (linear) represents the relative expression level of the probeset. Filled circles represent samples with expression levels in the linear detection range. Open circles represent an upper limit on gene expression in samples where the gene was below the probeset's detection limit. Open squares represent a lower limit on gene expression in samples where the probeset was saturated.

FIG. 4 depicts a graphical representation as shown in FIG. 3 except that the y-axis is log 2 based. Normal and cancerous tissue types are described along the horizontal axis. The names of cancerous tissues are preceded with ‘c_’ and the names of normal tissues are preceded with ‘n_’. ‘ns’ indicates a nonspecified tissue subtype.

FIG. 5 depicts a Western blot analysis showing AMIGO-2 protein isolated from six different cell lines. Relative semi-quantitative RT-PCR Ct levels are indicated in parentheses adjacent to names of four of the cell lines.

FIG. 6 depicts a graphical representation of AMIGO-2 mRNA levels in SW620 cells following administration of siRNAs. The y-axis is a relative scale and the numbers are arbitrary. UT=untransfected; Eg5=siRNA targeting Eg5 (an irrelevant gene); Neg Control=an siRNA sequence not homologous with any known gene; C315-1.2 through C315-4.3 are a panel of AMIGO-2 siRNAs.

FIG. 7A depicts a graphical representation of AMIGO-2 mRNA levels in Colo320 cells following administration of siRNAs. The y-axis is a relative scale and the numbers are arbitrary. Eg5=siRNA targeting Eg5 (an irrelevant gene); Neg Control=an siRNA sequence not homologous with any known gene; C315-1.2 and C315-4.3 are AMIGO-2 specific siRNAs.

FIG. 7B depicts a graphical representation of AMIGO-2 mRNA levels in HCT116 cells following administration of siRNAs. The y-axis is a relative scale and the numbers are arbitrary. Eg5=siRNA targeting Eg5 (an irrelevant gene); Neg Control=an siRNA sequence not homologous with any known gene; C315-1.2 and C315-4.3 are AMIGO-2 specific siRNAs.

FIG. 8A depicts a graphical representation of the effect of AMIGO-2-specific siRNAs on cell death of SW620 cells. The “Pos. Control” is an Eg5 siRNA targeting Eg5. The “Neg. control” is a siRNA sequence not homologous with any known gene. The y-axis measures the luminescence level, which is proportional to the number of dead cells.

FIG. 8B depicts a graphical representation of the effect of AMIGO-2-specific siRNAs on cell death of MRC9 cells. The “Pos. Control” is an Eg5 siRNA targeting Eg5. The “Neg. control” is a siRNA sequence not homologous with any known gene. The y-axis measures the luminescence level, which is proportional to the number of dead cells.

FIG. 9 depicts a panel of Western blots showing the effect of AMIGO-2-specific siRNAs on the expression and processing of AMIGO-2, PARP, M30, and tubulin proteins in AGS cells. The “Pos. Control” represents lysates from cells transfected with Eg5 siRNA. The “Neg. control” represents lysates from cells transfected with an siRNA sequence not homologous with any known gene.

FIG. 10A depicts a panel of Western blots showing the effect of AMIGO-2-specific siRNAs on the expression and processing of AMIGO-2, ERK, c-Myc, and tubulin proteins in SW620 cells. pERK is phosphorylated ERK protein. The “Neg. control” is as described in FIG. 9.

FIG. 10B depicts a graphical representation of the effect of AMIGO-2-specific siRNAs on c-MYC mRNA levels in SW620 cells. The “Pos. Control” is an Eg5 siRNA targeting Eg5. The “Neg. control” is a siRNA sequence not homologous with any known gene. The y-axis measures the relative expression level of mRNA as determined by qPCR.

FIG. 11 depicts a panel of Western blots showing the effect of AMIGO-2-specific siRNAs on the expression and processing of AMIGO-2, ERK, c-Myc, cyclin D1, and tubulin proteins in AGS cells. pERK is phosphorylated ERK protein. The lane labeled “anti-mitotic gene” represents lysates from cells transfected with Eg5 siRNA. The “Neg. control” represents lysates from cells transfected with a siRNA sequence not homologous with any known gene.

FIGS. 12A-12C depict effects of AMIGO-2 modulation on cJun expression. FIG. 12A is a panel of Western blots showing that cJun is downregulated in cells transfected with AMIGO-2 siRNA. “UT” represents an untransfected control sample, “DharmNeg” is a negative control siRNA sequence not homologous with any known gene, and C315-1.3si is an AMIGO-2 specific siRNA. FIG. 12B is a panel of Western blots showing that cJun expression is upregulated in cell lines stably transfected with AMIGO-2. FIG. 12C is a Western blot showing upregulation of cJun following exposure of AGS cells to the agonist anti-AMIGO-2 antibody MAB2080. “ISO” represents an isotype control.

FIG. 13 depicts a panel of Western blots depicting the effects of AMIGO-2 knockdown on cyclin B1 and cFosL1 expression in AGS and SW620 cells. “UT” represents an untransfected control sample, “DharmNeg” is a negative control siRNA sequence not homologous with any known gene, and C315-1.3si and C315-4.3si are AMIGO-2 specific siRNAs.

FIGS. 14A-C depict effects of AMIGO-2 modulation on cMyc expression. FIGS. 14A and 14B are panels of Western blots showing down-regulation of c-Myc following exposure of SW620 (FIG. 14A) and AGS (FIG. 14B) cells to AMIGO-2 siRNAs C315-1.3si and C315-4.3si. “UT” is an untransfected cell culture. “Neg.” represents a sample transfected with a siRNA sequence not homologous with any known gene. “Eg5” represents a sample transfected with siRNA targeting Eg5. FIG. 14C represents Western blots showing upregulation of cMyc in SW620 and AGS cells following exposure of cells to the anti-AMIGO-2 antibody MAB2080.

FIGS. 15A-15B depict genes concordantly down-regulated (FIG. 15A) and up-regulated (FIG. 15B) by AMIGO-2 modulators.

FIG. 16 depicts a panel of Western blots showing the effect of AMIGO-2-specific antibody MAB2080 (A) on the expression cJUN, cFosL1, and tubulin proteins in whole-cell lysate from SW620 cells (upper two panels). The series of Western blots in the lower panel depicts the effect MAB2080 (A) on the phosphorylation of ERK (PERK) as compared to total ERK in the cells. “I” or the “isotype control” represents a treatment of SW620 cells with non-specific mouse IgG.

### DETAILED DESCRIPTION

The inventors of the present application have discovered, inter alia, that AMIGO-2 is overexpressed in several cancers, including lung and colon cancer, and has restricted expression in normal tissues. Surprisingly, inhibition of AMIGO-2 inhibits cancer cell survival. Further, it has been found that inhibition of AMIGO-2 modulates levels of downstream markers including, for example, cyclin D1, cyclin B1, c-Myc, cJun, FosL1, VEGF, urokinase or ERK. Accordingly, the present invention provides methods and compositions for the treatment, diagnosis and imaging of cancer, in particular for the treatment, diagnosis and imaging of AMIGO-2-related cancers, as well as for the treatment of other diseases and disorders associated with aberrant expression of AMIGO-2. These and other aspects of the present invention are provided in the present application.

### DEFINITIONS

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook et al., Molecular Cloning: A Laboratory Manual (2nd Edition, 1989).

As used herein, the singular forms “a,” “an” and “the” include plural references unless the content clearly dictates otherwise. Thus, for example, reference to “an antibody” includes a mixture of two or more such antibodies.

As used herein, the term “about” refers to +/−30%, +/−20%, +/−10%, or +/−5% of a value.

As used herein, the term “AMIGO-2”, also known as Alivin 1 (ALI1) and DEGA, refers to an adhesion molecule with Ig-like domain 2. A nucleotide sequence of AMIGO-2 is set forth as SEQ ID NO:1 (GenBank Accession No. NM_{—}181847), and an amino acid sequence of AMIGO-2 is set forth as SEQ ID NO:2 (GenBank Accession No. NM_{—}181847). The term “AMIGO-2” also includes homologs, both nucleic acids and amino acids. In some embodiments, such AMIGO-2 nucleic acid and amino acids retain one or more activities of a native AMIGO-2 nucleic acid or amino acid.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some embodiments the subject is a human.

As used herein, “cancer” refers to primary or metastatic cancers. The term “cancer cells” refers to cells that are transformed. These cells can be isolated from a patient who has cancer, or be cells that are transformed in vitro to become cancerous. Cancer cells can be derived from many types of samples including any tissue or cell culture line. In some embodiments the cancer cells are hyperplasias, tumor cells, or neoplasms. In some embodiments, the cancer cells are isolated from lung tissue, bladder tissue, kidney tissue, colon tissue, breast tissue, uterine tissue, ovarian tissue, or pancreatic tissue. In some embodiments, the cancer cells are taken from established cell lines that are publicly available. In some embodiments, cancer cells are isolated from pre-existing patient samples or from libraries comprising cancer cells. In some embodiments, cancer cells are isolated and then implanted in a different host, e.g., in a xenograft. In some embodiments cancer cells are transplanted and used in a SCID mouse model. In some embodiments, the cancer is lung or colon cancer. In some embodiments, the cancer is a cancer other than gastric cancer.

As used herein, the term “transformed” refers to any alteration in the properties of a cell that is stably inherited by its progeny. In some embodiments, “transformed” refers to the change of normal cell to a cancerous cell, e.g., one that is capable of causing tumors. In some embodiments, a transformed cell is immortalized. Transformation can be caused by a number of factors, including overexpression of a receptor in the absence of receptor phosphorylation, viral infection, mutations in oncogenes and/or tumor suppressor genes, and/or any other technique that changes the growth and/or immortalization properties of a cell.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, or the like.

As used herein, the term “metastasis” refers to a cancer which has spread to a site distant from the origin of the cancer, e.g. from the primary tumor. Sites of metastasis include without limitation, the bone, lymph nodes, lung, liver, and brain.

As used herein, the term “angiogenesis” refers to the development of blood vessels in a patient.

As used herein, the term “clinical endpoint” refers to a measurable event indicative of cancer. Clinical endpoints include without limitation, time to first metastasis, time to subsequent metastasis, size and/or number of metastases, size and/or number of tumors, location of tumors, aggressiveness of tumors, quality of life, pain and the like. Those skilled in the art are credited with the ability to determine and measure clinical endpoints. Methods of measuring clinical endpoints are known to those of skill in the art.

As used herein, the term “sample” refers to biological material from a patient. The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

As used herein, the term “biological molecule” includes, but is not limited to, polypeptides, nucleic acids, and saccharides.

As used herein, the term “modulating” refers to a change in the quality or quantity of a gene, protein, or any molecule that is inside, outside, or on the surface of a cell. The change can be an increase or decrease in expression or level of the molecule. The term “modulates” also includes changing the quality or quantity of a biological function/activity including, without limitation, cell signaling activity, cell cycle regulation, a kinase activity, a serine/threonine kinase activity, a cell-cell interaction activity, an activity affecting ploidy, chromosomal stability, tumorigenicity, cell motility, metastasis, cancer cell survival, cancer cell growth, proliferation, progression through the cell cycle, anchorage-independent growth, localization of AMIGO-2 protein to the cell-membrane, cell-to-cell interactions including interactions between AMIGO-2 and one or both of AMIGO-1 (GenBank Accession No. NM_{—}020703; amino acid set forth as SEQ ID NO:63) or AMIGO-3 (GenBank Accession No. NM_{—}198722, amino acid sequence set forth as SEQ ID NO:64), levels of cytoplasmic phosphorylated AMIGO-2 protein, angiogenesis, neuronal outgrowth or cell death.

As used herein, the term “modulator” refers to a composition that modulates one or more physiological or biochemical events associated with cancer. In some embodiments the modulator inhibits one or more biological activities associated with cancer. In some embodiments the modulator is a small molecule, an antibody, a mimetic, a decoy or an oligonucleotide. In some embodiments the modulator acts by blocking ligand binding or by competing for a ligand-binding site. In some embodiments the modulator acts independently of ligand binding. In some embodiments the modulator does not compete for a ligand binding site. In some embodiments the modulator blocks expression of a gene product involved in cancer. In some embodiments the modulator blocks a physical interaction of two or more biomolecules involved in cancer. In some embodiments modulators of the invention inhibit one or more AMIGO-2 activities selected from the group consisting of cell signaling activity, cell cycle regulation, a kinase activity, a serine/threonine kinase activity, a cell-cell interaction activity, an activity affecting ploidy, chromosomal stability, tumorigenicity, cell motility, metastasis, cancer cell survival, cancer cell growth, proliferation, progression through the cell cycle, anchorage-independent growth, localization of AMIGO-2 protein to the cell-membrane, cell-to-cell interactions including interactions between AMIGO-2 and one or both of AMIGO-1 or AMIGO-3, levels of cytoplasmic phosphorylated AMIGO-2 protein, angiogenesis or neuronal outgrowth. In some embodiments, the AMIGO-2 modulator, inhibits AMIGO-2 expression. In some embodiments, the modulator inhibits progression of dividing cells into the G2/M stage of the cell cycle.

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term are biopolymeric products that are made using an RNA gene product as a template (i.e. cDNA of the RNA). A gene product may be made enzymatically, recombinantly, chemically, or within a cell to which the gene is native. In some embodiments, if the gene product is proteinaceous, it exhibits a biological activity. In some embodiments, if the gene product is a nucleic acid, it can be translated into a proteinaceous gene product that exhibits a biological activity.

“Modulation of AMIGO-2 activity”, as used herein, refers to an increase or decrease in an AMIGO-2 activity that can be a result of, for example, interaction of an agent with an AMIGO-2 polynucleotide or polypeptide, inhibition of AMIGO-2 transcription and/or translation (e.g., through antisense or siRNA interaction with the AMIGO-2 gene or AMIGO-2 gene expression product, through modulation of transcription factors that facilitate AMIGO-2 expression), and the like. For example, modulation of an AMIGO-2 activity refers to an increase in a biological activity or a decrease in a biological activity. Modulation of AMIGO-2 activity also refers to increasing or decreasing one or more AMIGO-2 phenotypes. AMIGO-2 activity can be assessed by means including, without limitation, assessing AMIGO-2 polypeptide levels, or by assessing AMIGO-2 transcription levels. Comparisons of AMIGO-2 activity can also be accomplished by measuring levels of an AMIGO-2 downstream marker, measuring chromosomal stability, measuring kinase activity, measuring tumorigenicity, measuring metastasis, measuring AMIGO-2 signaling, measuring AMIGO-2 mediated cell adhesion, measuring AMIGO-2 mediated cancer cell apoptosis, measuring ERK phosphorylation, measuring cancer cell growth, measuring tumor formation, measuring cyclin production, measuring cell proliferation, measuring cancer cell growth, measuring anchorage-independent growth, measuring cell cycle regulation, measuring neuronal guidance, measuring cancer cell motility, measuring localization of AMIGO-2 protein to the cell membrane, measuring cell-to-cell interactions including interactions between AMIGO-2 and one or both of AMIGO-1 or AMIGO-3, measuring levels of cytoplasmic phosphorylated AMIGO-2 protein, measuring angiogenesis, and measuring cell death, among others.

In some embodiments, inhibition of AMIGO-2 activity is of particular interest. As used herein, the term “inhibit” refers to a reduction, decrease, inactivation or down-regulation of an activity or quantity. For example, in the context of the present invention, AMIGO-2 modulators may inhibit one or more of tumorigenicity; cancer cell motility; cell adhesion; metastasis; cancer cell survival; kinase activity; proliferation; anchorage-independent growth; cancer cell motility; localization of AMIGO-2 protein to the cell-membrane; interactions between AMIGO-2 and one or both of AMIGO-1 or AMIGO-3; neuronal guidance; levels of cytoplasmic phosphorylated AMIGO-2 protein; levels of phosphorylated ERK; and angiogenesis. Inhibition of such activities may be at least 25%, at least 50%, at least 75%, at least 80%, at least 90%, at least 95%, at least 97%, at least 98%, at least 99%, or 100%, as compared to a control. Those of skill in the art are credited with the ability to measure AMIGO-2 modulation; a non-limiting list of exemplary assays is set forth below.

Accordingly, as used herein, the term “inhibition of AMIGO-2” refers to a reduction, decrease, inactivation or down-regulation of one or more AMIGO-2-mediated biological activities. Inhibition of an “AMIGO-2 biological activity” refers to a reduction, decrease, inactivation, or down-regulation of, for example, tumorigenicity; cancer cell motility; cell adhesion; metastasis; cancer cell survival; kinase activity; proliferation; anchorage-independent growth; cancer cell motility; localization of AMIGO-2 protein to the cell-membrane; interactions between AMIGO-2 and one or both of AMIGO-1 or AMIGO-3; neuronal guidance; levels of cytoplasmic phosphorylated AMIGO-2 protein; levels of phosphorylated ERK; or angiogenesis. Inhibition of such activities may be at least 25%, at least 50%, at least 75%, at least 80%, at least 90%, at least 95%, at least 97%, at least 98%, at least 99%, or 100%, as compared to a control.

In some embodiments, modulation of AMIGO-2 activities that activate or result in an increase of AMIGO-2 activity is of particular interest. AMIGO-2 modulators may inhibit one or more of ploidy and cell death. Activation, upregulation or increases in the AMIGO-2 activity may be at least 125%, at least 150%, at least 200%, at least 250%, at least 300%, at least 500% as compared to a control. For example, an AMIGO-2 modulator that increases cell death 200% has increased cell death two-fold as compared to a control lacking the AMIGO-2 modulator.

As used herein, the term “differentially expressed in a cancer cell” and “a polynucleotide that is differentially expressed in a cancer cell” are used interchangeably herein, and refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source, or between one cell in situ and a second cell removed from its tissue source. In some embodiments, the gene is upregulated in the cancer gene as compared to the normal cell.

An AMIGO-2 associated-cancer is “inhibited” if at least one symptom of the cancer is alleviated, terminated, slowed, or prevented. As used herein, an AMIGO-2 associated-cancer is also “inhibited” if recurrence or metastasis of the cancer is reduced, slowed, delayed, or prevented.

As used herein, the phrase “inhibits AMIGO-2 mediated cell adhesion” refers to a decrease, reduction, or abolition of cell-to-cell adhesion in the presence of an AMIGO-2 modulator wherein at least one cell differentially expresses AMIGO-2. In this context, AMIGO-2 mediated cell adhesion can be decreased by an AMIGO-2 modulator at least 25%, at least 50%, at least 75%, at least 85%, at least 90%, at least 95%, up to 100% relative to AMIGO-2 mediated cell adhesion in the absence of an AMIGO-2 modulator. Comparisons of AMIGO-2 mediated cell adhesion can be accomplished by measuring, for example, by labeling the cells of interest, incubating them with a population of unlabeled cells adhering to a substrate, and washing to separate the adherent from the non-adherent populations. In this manner, cell adhesion is determined by measuring the amount of label retained on the substrate. Examples of assay systems include, but are not limited to labeling with fluorescent probes such as calcein AM, CFMDA (5-chloromethylfluorescein diacetate), 5(6)-CFDA-SE [5-(and-6)-carboxyfluorescein diacetate, succinimidyl ester] and measuring fluorescence in a fluorescence plate reader or via flow cytometry.

As used herein, the phrase “inhibits cancer cell growth” refers to a decrease, reduction, or abolition of cancer cell growth in the presence of an AMIGO-2 modulator wherein the cell expresses AMIGO-2. In some embodiments the cells differentially express AMIGO-2 relative to other normal cells and/or relative to other cancer cells. In this context, cell growth can be decreased by an AMIGO-2 modulator at least 25%, at least 50%, at least 75%, at least 85%, at least 90%, at least 95%, up to 100% relative to cancer cell growth in the absence of an AMIGO-2 modulator. Comparisons of cancer cell growth can be accomplished using, for example, MTT assay (for example, the Vybrant® MTT Cell Proliferation Assay Kit (Invitrogen)); BrdU incorporation (for example, the Absolute-S SBIP assay (Invitrogen)); measuring intracellular ATP levels (for example using ATPLite™-M, 1,000 Assay Kit (PerkinElmer) or ATP Cell Viability Assay Kit (BioVision)); DiOc18 assay, a membrane permeable dye (Invitrogen); Glucose-6-phosphate dehydrogenase activity assay (for example, the Vibrant cytotoxicity assay (Invitrogen)); or measuring cellular LDH activity.

As used herein, the phrase “inhibits tumor formation” refers to a decrease, reduction, or abolition of tumor formation in the presence of an AMIGO-2 modulator wherein the tumor comprises cells that differentially express AMIGO-2. In this context, tumor formation can be decreased by an AMIGO-2 modulator at least 25%, at least 50%, at least 75%, at least 85%, at least 90%, at least 95%, and up to 100% relative to tumor formation in the absence of an AMIGO-2 modulator. Comparisons of tumor formation can be accomplished using, for example, cell based assays (for example colony formation in soft agar); in vivo models of tumor formation typically relying upon injecting the cells of interest into animals (for example, athymic mice or rats, irradiated mice or rats; inoculation into immunologically privileged sites such as brain, cheek pouch or eye; inoculation of syngeneic animals), and monitoring the size of the mass after a defined time period.

As used herein, the phrase “inhibits cyclin D1” refers to a decrease, reduction, or abolition of AMIGO-2 mediated cyclin production. In this context, AMIGO-2 mediated cyclin production can be decreased by an inhibitory agent at least 25%, at least 50%, at least 75%, at least 85%, at least 90%, at least 95%, up to 100% relative to AMIGO-2 mediated cyclin production in the absence of an AMIGO-2 modulator. Comparisons of cyclin production can be accomplished by measuring, for example, cyclin mRNA levels via RT-PCR or northern blotting; cyclin polypeptide levels via immunoblotting, immunoprecipitation or ELISA; or using functional assays, including co-immunoprecipitation assays to measure levels of cyclin that are complexed with cyclin regulators such as cyclin-dependent kinases (CDK's) using for example antibodies that target CDK, p21WAF1, p27 KIP-1; and measuring phosphorylation of cyclins by the CDK's can be assayed through radiolabeling and immunoprecipitation analysis or FRET-based methods, for example, CDK2/Cyclin A Assay Kit (Molecular Devices).

As used herein, the phrase “inhibits cyclin B1” refers to a decrease, reduction, or abolition of AMIGO-2 mediated cyclin production. In this context, AMIGO-2 mediated cyclin production can be decreased by an inhibitory agent at least 25%, at least 50%, at least 75%, at least 85%, at least 90%, at least 95%, up to 100% relative to AMIGO-2 mediated cyclin production in the absence of an AMIGO-2 modulator. Comparisons of cyclin production can be accomplished according to methods described above for cyclin D1.

As used herein, an “inhibition of FosL1” refers to a decrease, reduction, or abolition of AMIGO-2-mediated FosL1 production. In this context, AMIGO-2-mediated FosL1 production can be decreased by an inhibitory agent at least 25%, at least 50%, at least 75%, at least 85%, at least 90%, at least 95%, up to 100% relative to AMIGO-2-mediated FosL1 production in the absence of an AMIGO-2 modulator. Comparisons of FosL1 production can be accomplished by measuring, for example, FosL1 mRNA levels via RT-PCR or northern blotting; or FosL1 polypeptide levels via immunoblotting, immunoprecipitation, ELISA, or immunohistochemistry. Examples of suitable methods of measuring FosL1 expression (e.g., mRNA or protein expression) are set forth in the present Examples and also described in, e.g., Matsuo et al. (2000) Nature Genet. 24:184-187; and Sahin et al. (2005) Pancreas 30(2):158-167.

As used herein, “inhibition of c-Myc” refers to a decrease, reduction, or abolition of AMIGO-2-mediated c-Myc production. In this context, AMIGO-2-mediated c-Myc production can be decreased by an inhibitory agent at least 25%, at least 50%, at least 75%, at least 85%, at least 90%, at least 95%, up to 100% relative to AMIGO-2-mediated c-Myc production in the absence of an AMIGO-2 modulator. Comparisons of c-Myc production can be accomplished by measuring, for example, c-Myc mRNA levels via RT-PCR or northern blotting; or c-Myc polypeptide levels via immunoblotting, immunoprecipitation, ELISA, or immunohistochemistry. Alternatively, or in addition, c-Myc can be measured “functionally,” for example, by the ability of c-Myc transcription factor to promote transcription of a target gene. The target gene can be an endogenous gene or can be an exogenous transgene (i.e., a reporter gene). Measuring expression of the target gene mRNA or protein can be accomplished using any of the methods described herein. In some instances, the reporter gene can be an enzyme with measurable activity (e.g., luciferase, chloramphenicol acetyltransferase, alkaline phosphatase, or horseradish peroxidase) or a detectable protein (e.g., green fluorescent protein or red fluorescent protein). Examples of suitable methods of measuring c-Myc expression (e.g., mRNA or protein expression) are both known in the art and amply set forth in the present Examples. Methods of monitoring reporter gene expression, particularly enzyme or detectable reporter genes as described above, are well known in the art and described in, e.g., Cziepluch et al. (1993) Oncogene 8(10):2833-8.

As used herein, “inhibition of c-Jun” refers to a decrease, reduction, or abolition of AMIGO-2-mediated c-Jun production. In this context, AMIGO-2-mediated c-Jun production can be decreased by an inhibitory agent at least 25%, at least 50%, at least 75%, at least 85%, at least 90%, at least 95%, up to 100% relative to AMIGO-2-mediated c-Jun production in the absence of an AMIGO-2 modulator. Comparisons of c-Jun production can be accomplished by measuring, for example, c-Jun in RNA levels via RT-PCR or northern blotting; or c-Jun polypeptide levels via immunoblotting, immunoprecipitation, ELISA, or immunohistochemistry. Alternatively, or in addition, c-Jun can be measured “functionally,” for example, by the ability of c-Jun transcription factor to promote transcription of a target gene. The target gene can be an endogenous gene or can be an exogenous transgene (i.e., a reporter gene). Measuring expression of the target gene mRNA or protein can be accomplished using any of the methods described herein. Examples of suitable methods of measuring c-Jun expression (e.g., mRNA or protein expression) are both known in the art, amply set forth in the present Examples, and further described in, e.g., Kharbanda et al. (1991) Biochemistry 30:7947-7952 and Kayahara et al. (2005) Mol. Cell. Biol. 25(9):3784-3792. Methods of monitoring reporter gene expression, particularly enzyme or detectable reporter genes as described above, are well known in the art and described above.

As used herein, the phrase “inhibits ERK phosphorylation” refers to a decrease, reduction, or abolition of AMIGO-2 mediated ERK phosphorylation. In this context, AMIGO-2 mediated ERK phosphorylation can be decreased by an AMIGO-2 modulator at least 25%, at least 50%, at least 75%, at least 85%, at least 90%, at least 95%, up to 100% relative to AMIGO-2 mediated ERK phosphorylation in the absence of an AMIGO-2 modulator. Comparisons of ERK phosphorylation can be assessed using phosphorylation assays known to those of skill in the art.

While not limited by any particular theory or mechanism of action, since phosphorylation of ERK, generally leads to its activation, the phrase “inhibits ERK phosphorylation” can, in some embodiments, also refer to a decrease, reduction, or abolition of AMIGO-2 mediated phosphorylation of one or more ERK substrates by phosphorylated ERK. ERK substrates include, but are in no way limited to, TEX-1, ELK1, Paxillin, Bcl-2, SOS, or SP1. Methods for monitoring the kinase activity of ERK on its substrates are known in the art and are described in, e.g., Mechant et al. (1999) BBRC 254:454-461; Chemiack et al. (1994) J. Biol. Chem. 269:4717-4720; Cano et al. (1995) J. Cell Sci. 108:3599-3609; Garcia et al. (2004) EMBO J. 21:5151-5163; and Tamura et al. (2004) FEBS Lett. 569:249-255.

As used herein, the phrase “inhibits cancer cell survival” refers to a decrease or reduction of survival of cancer cells that express AMIGO-2. In some embodiments the term “inhibits cancer cell survival” refers to effecting apopotosis of cancer cells that express AMIGO-2. In some embodiments the cancer cells differentially express AMIGO-2 relative to other normal cells and/or relative to other cancer cells. In this context, AMIGO-2 expressing cancer cell survival can be decreased by an inhibitory agent at least 25%, at least 50%, at least 75%, at least 85%, at least 90%, at least 95%, up to 100% relative to cancer cell survival in the absence of an AMIGO-2 modulator and/or in a normal cell.

As used herein, the phrase “inhibits AMIGO-2 signaling” refers to decreasing, reducing, or abolishing the effect of AMIGO-2 on downstream members of cellular signaling cascades that include AMIGO-2. Cellular signaling cascades that include AMIGO-2 include pathways that mediate cell growth and survival. In some embodiments the pathways that mediate cell growth and survival are downstream of activated growth factor pathways, such as the EGFR pathway or the beta-catenin pathway. In some embodiments the pathway is a mutated beta-catenin pathway which results in stabilization of beta-catenin, among others. In some embodiments, inhibition of AMIGO-2 signaling up-regulates one or more downstream markers. In some embodiments, inhibition of AMIGO-2 signaling down-regulates one or more downstream markers.

Inhibition of AMIGO-2 signaling can be determined by measuring polypeptide or polynucleotide levels of downstream members of the cellular signaling pathway. Those of skill in the art are credited with the ability of measuring AMIGO-2 polypeptide and/or polynucleotide levels. The art-skilled can also measure levels of AMIGO-2 downstream markers.

As used herein, the phrase “inhibits cell-cell interaction” refers to reducing or eliminating an interaction between two or more cells that express AMIGO-2. In some embodiments, the interaction between the cells leads to a cell signal. Cell-cell interaction can be detected via a number of methods known to those of skill in the art, including, without limitation, the observation of membrane exchange between co-cultured, pre-labeled cells, labeled, for example, with different fluorescent membrane stains including PKH26 and PKH67 (Sigma).

As used herein, the phrases “inhibits proliferation” refers to reducing or eliminating AMIGO-2-mediated proliferation and can be measured via a number of methods known to those of skill in the art. Cell proliferation assays include, without limitation, MTT assays (for example, the Vybrant® MTT Cell Proliferation Assay Kit (Invitrogen)); BrdU incorporation assays (for example, the Absolute-S SBIP assay (Invitrogen)); measuring intracellular ATP levels (commercial versions of the assay include ATPLite™-M, 1,000 Assay Kit (PerkinElmer) and ATP Cell Viability Assay Kit (BioVision)); DiOc18 assay, a membrane permeable dye (Invitrogen); Glucose-6-phosphate dehydrogenase activity assay (for example, the Vibrant cytotoxicity assay (Invitrogen)); measuring cellular LDH activity; and ³H-thymidine incorporation and the Cell Titer Glo Assay (Promega).

As used herein, the phrase “inhibits angiogenesis” refers to reducing or eliminating AMIGO-2-mediated angiogenesis. Angiogenesis can be detected via a number of methods known to those of skill in the art, including, without limitation, cell proliferation assays, cell migration assays, cell differentiation assays, organ culture (ex vivo) assays, chick chorioallantoic membrane (CAM) assays, corneal angiogenesis assays, Matrigel plug assays, and tumor volume assays in SCID mice, nude mice, or C57BL mice.

Cell migration assays include, without limitation, blind-well chemotaxis chamber, e.g., modified Boyden chamber and the Phagokinetic track assay. Cell differentiation assays include, without limitation, tube formation in collagen, fibrin clots, or Matrigel, followed by electron microscopy. Organ culture (ex vivo) assays include, without limitation, rat aortic ring assay and chick aortic arch assay.

As used herein, the phrase “inhibits progression through the cell cycle” refers to slowing or stalling the cell division. Cell-cycle progression can be assayed by bromodeoxyuridine (BRDU) incorporation. Such assays identify a cell population undergoing DNA synthesis by incorporation of BRDU into newly synthesized DNA. Newly-synthesized DNA may then be detected using an anti-BRDU antibody (Hoshino et al., 1986, int. J. Cancer 38, 369; Campana et al., 1988, J. Immunol. Meth. 107, 79), or by other means. Cell proliferation can also be assayed by phospho-histone H3 staining, which identifies a cell population undergoing mitosis by phosphorylation of histone H3. Phosphorylation of histone H3 at serine 10 is detected using an antibody specific to the phosphorylated form of the serine 10 residue of histone H3. (Chadlee, D. N. 1995, J. Biol. Chem 270:20098-105). Cell proliferation can also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA synthesis. In this assay, cells synthesizing DNA will incorporate [³H]-thymidine into newly synthesized DNA. Incorporation can then be measured by standard techniques such as by counting of radioisotope in a scintillation counter (e.g., Beckman L S 3800 Liquid Scintillation Counter). Another proliferation assay uses the dye Alamar Blue (available from Biosource International), which fluoresces when reduced in living cells and provides an indirect measurement of cell number (Voytik-Harbin S L et al., 1998, In Vitro Cell Dev Biol Anim 34:239-46). Yet another proliferation assay, the MTS assay, is based on in vitro cytotoxicity assessment of industrial chemicals, and uses the soluble tetrazolium salt, MTS. MTS assays are commercially available and include the Promega CellTiter 96® AQueous Non-Radioactive Cell Proliferation Assay (Cat.# G5421). Cell proliferation can also be assayed by colony formation in soft agar (Sambrook et al., Molecular Cloning, Cold Spring Harbor (1989)). Cell proliferation may also be assayed by measuring ATP levels as indicator of metabolically active cells. Such assays are commercially available and include Cell Titer-Glo™ (Promega). Cell cycle proliferation can also be assayed by flow cytometry (Gray J W et al. (1986) Int J Radiat Biol Relat Stud Phys Chem Med 49:237-55). Cells may be stained with propidium iodide and evaluated in a flow cytometer to measure accumulation of cells at different stages of the cell cycle.

An “AMIGO-2 downstream marker”, as used herein, is a gene or activity which exhibits altered level of expression in a cancer tissue or cancer cell compared to its expression level in normal or healthy tissue, or is a property altered in the presence of an AMIGO-2 modulator. In some embodiments, the downstream markers exhibit altered levels of expression when AMIGO-2 is perturbed with an AMIGO-2 modulator of the present invention. AMIGO-2 downstream markers include, without limitation, cyclin D1, cyclin B1, c-Myc, VEGF, urokinase, cJun, FosL1, or ERK. In some embodiments cleavage of PARP1 is a downstream marker of AMIGO-2 activity.

As used herein, the phrase “increasing cancer cell apoptosis” refers to increasing apoptosis of cancer cells that express AMIGO-2 in the presence of an AMIGO-2 modulator. In this context, cancer cell apoptosis can be increased by an AMIGO-2 modulator by at least 25%, at least 50%, at least 75%, at least 85%, at least 90%, at least 95%, up to 100% relative to cancer cell apoptosis in the absence of an AMIGO-2 modulator. In some embodiments the cancer cells differentially express AMIGO-2 relative to other normal cells and/or relative to other cancer cells. Comparisons of cancer cell apoptosis can be accomplished by measuring, for example, DNA fragmentation, caspase activity, loss of mitochondrial membrane potential, increased production of reactive oxygen species (ROS), intracellular acidification, chromatin condensation, phosphatidyl serine (PS) levels at the cell surface, and increased cell membrane permeability.

DNA fragmentation can be measured, for example, with the TUNEL assay (terminal deoxynucleotide transferase dUTP nick end labeling). Commercial versions of the assay are widely available, for example, APO-BrdU™ TUNEL Assay Kit (Invitrogen), APO-DIRECT™ Kit (BD Biosciences Pharmingen) and ApoAlert™ DNA Fragmentation Assay Kit (Clontech, a Takara Bio Company).

Caspase activity can be monitored via fluorogenic, chromogenic and luminescent substrates specific for particular caspases. Commercial assay kits are available for at least caspases 1, 2, 3, 6, 7, 8 and 9. (See, for example, Invitrogen, Chemicon, CalBiochem, BioSource International, Biovision).

Loss of mitochondrial membrane potential can be measured with fluorescent dyes that differentially accumulate in healthy active mitochondria. One non-limiting example is the MitoTracker Red system from Invitrogen.

Production of reactive oxygen species (ROS) can be measured with fluorescent dyes including, for example, H2DCFDA (Invitrogen).

Intracellular acidification can be measured with fluorescent or chromogenic dyes.

Chromatin condensation can be measured with fluorescent dyes including, for example, Hoechst 33342.

Phosphatidyl serine (PS) levels can be measured at the cell surface. For example, Annexin V has a high affinity for PS. Numerous commercially available assays are suitable to monitor the binding of labeled AnnexinV to the cell surface.

Cell membrane permeability can be measured using dyes, such as the fluorescent dye, YO-PRO-1 (Invitrogen) which can enter apoptotic, but not necrotic cells.

As used herein, the term “up-regulates” refers to an increase, activation or stimulation of an activity or quantity.

As used herein, the term “N-terminus” refers to the first 10 amino acids of a protein.

As used herein, the term “C-terminus” refers to the last 10 amino acids of a protein.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “extracellular domain” refers to the portion of a molecule that is outside or external to a cell. In the context of the present invention, an N-terminal extracellular domain refers to the extracellular domain that is present at the N-terminus of the molecule immediately before the first transmembrane domain. In the context of an extracellular domain between two transmembrane (TM) domains, for example between TM 2&3, extracellular domain refers to that portion of AMIGO-2 external to the cell membrane between the second and third transmembrane domains of AMIGO-2.

As used herein, the term “ligand binding domain” refers to any portion or region of a receptor retaining at least one qualitative binding activity of a corresponding native sequence of AMIGO-2.

The term “region” refers to a physically contiguous portion of the primary structure of a biomolecule. In the case of proteins, a region is defined by a contiguous portion of the amino acid sequence of that protein. In some embodiments, a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, fragments of biomolecules have a biological activity. In the context of the present invention, AMIGO-2 polypeptide fragments do not comprises the entire AMIGO-2 polypeptide sequence set forth in SEQ ID NO:2. In some embodiments, AMIGO-2 fragments retain one or more activities of native AMIGO-2.

As used herein, the phrase “AMIGO-2-related cells/tumors/samples” and the like refers to cells, samples, tumors or other pathologies that are characterized by differential expression of AMIGO-2 relative to non-cancerous and/or non-metastatic cells, samples, tumors, or other pathologies. In some embodiments, AMIGO-2-related cells, samples, tumors or other pathologies are characterized by increased AMIGO-2 expression relative to non-metastatic cells, samples, tumors, or other pathologies.

As used herein, the term “antibody” refers to monoclonal and polyclonal antibodies, single chain antibodies, chimeric antibodies, bifunctional/bispecific antibodies, humanized antibodies, human antibodies, and complementary determining region (CDR)-grafted antibodies, that are specific for the target protein or fragments thereof, and also include antibody fragments, including Fab, Fab′, F(ab′)2, scFv, Fv, camelbodies, or microantibodies. The term “antibody” further includes in vivo therapeutic antibody gene transfer.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J. Mol. Biol., 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, in some embodiments comprising the antigen-binding or variable region thereof. Examples of antibody fragments include Fab, Fab′, F(ab′)2, and Fv fragments; diabodies; linear antibodies (Zapata et al., Protein Eng. 8(10): 1057-1062 [1995]); single-chain antibody molecules; and multispecific antibodies formed from antibody fragment(s).

An “intact” antibody is one that comprises an antigen-binding variable region as well as a light chain constant domain (C_{L}) and heavy chain constant domains, C_{H1}, C_{H2} and C_{H3}. The constant domains may be native sequence constant domains (e.g. human native sequence constant domains) or amino acid sequence variants thereof. In some embodiments the intact antibody has one or more effector functions.

Antibody “effector functions” refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g. B cell receptor, BCR), etc.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Human effector cells” are leukocytes that express one or more FcRs and perform effector functions. In some embodiments the cells express at least FcγRIII and perform ADCC effector function. Examples of human leukocytes that mediate ADCC include peripheral blood mononuclear cells (PBMC), natural killer (NK) cells, monocytes, cytotoxic T cells and neutrophils. The effector cells may be isolated from a native source thereof, e.g. from blood or PBMCs as described herein.

The terms “Fc receptor” or “FcR” are used to describe a receptor that binds to the Fc region of an antibody. In some embodiments the FcR is a native sequence human FcR. Moreover, in some embodiments the FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review in Daron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

As used herein, the term “epitope” refers to an antigenic determinant of a polypeptide. In some embodiments an epitope may comprise 3 or more amino acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such amino acids. Methods of determining the spatial conformation of amino acids are known in the art, and include, for example, x-ray crystallography and 2-dimensional nuclear magnetic resonance.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which are incorporated herein by reference.

A wide variety of antibody/immunoglobulin frameworks or scaffolds can be employed so long as the resulting polypeptide includes at least one binding region that is specific for the target protein. Such frameworks or scaffolds include the 5 main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

One can generate non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target. Such compounds are known herein as “polypeptides comprising a target-specific binding region”. Known non-immunoglobulin frameworks or scaffolds include, but are not limited to, Adnectins (fibronectin) (Compound Therapeutics, Inc., Waltham, Mass.), ankyrin (Molecular Partners AG, Zurich, Switzerland), domain antibodies (Domantis, Ltd (Cambridge, Mass.) and Ablynx nv (Zwijnaardc, Belgium)), lipocalin (Anticalin) (Pieris Proteolab AG, Freising, Germany), small modular immuno-pharmaceuticals (Truhion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Sell Proteins GmbH, Halle, Germany).

(iii) Maxybodies/Avimers-Avidia

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, US Patent Publications 20040175756; 20050053973; 20050048512; and 20060008844.

The term “antagonist” is used in the broadest sense, and includes any molecule that partially or fully blocks, inhibits, or neutralizes a biological activity of a tumor cell antigen disclosed herein. In a similar manner, the term “agonist” is used in the broadest sense and includes any molecule that mimics a biological activity of a tumor cell antigen disclosed herein. Suitable agonist or antagonist molecules specifically include agonist or antagonist antibodies or antibody fragments, fragments or amino acid sequence variants of tumor cell antigens, peptides, antisense oligonucleotides, small organic molecules, etc. Methods for identifying agonists or antagonists of a tumor cell antigen may comprise contacting a tumor cell expressing the antigen of interest with a candidate agonist or antagonist molecule and measuring a delectable change in one or more biological activities normally associated with the tumor cell antigen. The antagonist may also be a peptide generated by rational design or by phage display (see, e.g., WO98/35036 published 13 Aug. 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

As used herein, the term “oligonucleotide” refers to a series of linked nucleotide residues. Oligonucleotides include without limitation, antisense and siRNA oligonucleotides. Oligonucleotides comprise portions of a DNA sequence and have at least about 10 nucleotides and as many as about 500 nucleotides. In some embodiments oligonucleotides comprise from about 10 nucleotides to about 50 nucleotides, from about 15 nucleotides to about 30 nucleotides, and from about 20 nucleotides to about 25 nucleotides. Oligonucleotides may be chemically synthesized and can also be used as probes. In some embodiments oligonucleotides are single stranded. In some embodiments oligonucleotides comprise at least one portion which is double stranded. In some embodiments the oligonucleotides are antisense oligonucleotides (ASO). In some embodiments the oligonucleotides are RNA interference oligonucleotides (RNAi oligonucleotides).

As used herein, the term “antisense oligonucleotide” refers to an unmodified or modified nucleic acid having a nucleotide sequence complementary to an AMIGO-2 polynucleotide sequence including polynucleotide sequences associated with the transcription or translation of AMIGO-2 (e.g., a promoter of an AMIGO-2 polynucleotide), where the antisense polynucleotide is capable of hybridizing to an AMIGO-2 polynucleotide sequence. Of particular interest are antisense polynucleotides capable of inhibiting transcription and/or translation of AMIGO-2 polypeptide-encoding polynucleotide either in vitro or in vivo.

As used herein, the terms “siRNA oligonucleotides”, “RNAi oligonucleotides”, “short interfering RNA”, or “siRNA” are used interchangeably and refer to oligonucleotides that work through post-transcriptional gene silencing, also known as RNA interference (RNAi). The terms refer to a double stranded nucleic acid molecule capable of RNA interference “RNAi”, (see Kreutzer et al., WO 00/44895; Zernicka-Goetz et al. WO 01/36646; Fire, WO 99/32619; Mello and Fire, WO 01/29058). siRNA molecules are generally RNA molecules but further encompass chemically modified nucleotides and non-nucleotides. SiRNA gene-targeting experiments have been carried out by transient siRNA transfer into cells (achieved by such classic methods as liposome-mediated transfection, electroporation, or microinjection). Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi.

As used herein, the term “decoy receptor” refers to a receptor comprising at least a portion of a polypeptide, mimetic, or other macromolecule capable of binding an AMIGO-2 ligand. As used herein, the term “therapeutically effective amount” is meant to refer to an amount of a medicament which produces a medicinal effect observed as reduction or reverse in one or more clinical endpoints, growth and/or survival of cancer cell, or metastasis of cancer cells in an individual when a therapeutically effective amount of the medicament is administered to the individual. Therapeutically effective amounts are typically determined by the effect they have compared to the effect observed when a composition which includes no active ingredient is administered to a similarly situated individual. The precise effective amount for a subject will depend upon the subject's size and health, the nature and extent of the condition, and the therapeutics or combination of therapeutics selected for administration. However, the effective amount for a given situation is determined by routine experimentation and is within the judgment of the clinician.

As used herein, the terms “in combination with” or “in conjunction with” refer to administration of the AMIGO-2 modulators of the invention with other therapeutic regimens.

As used herein, the term “susceptible” refers to patients for whom AMIGO-2 therapy is an acceptable method of treatment, i.e., patients who are likely to respond positively. In some embodiments, cancer patients susceptible to AMIGO-2 therapy express high levels of AMIGO-2 relative to those patients not susceptible to AMIGO-2 therapy. In some embodiments, cancer patients who are not good candidates for AMIGO-2 therapy include cancer patients with tumor samples that tack or have lower levels of AMIGO-2 in or on their cancer cells. In some embodiments, patients having a higher proportion of AMIGO-2 localized to the cell membrane as compared to AMIGO-2 localized to other areas of the cancer cells indicates that such patients are more susceptible to AMIGO-2 therapy than those with a lower proportion of cell membrane-localized AMIGO-2 compared to non-cell membrane localized AMIGO-2. In some embodiments a ratio of AMIGO-2 localized to the cell membrane compared to AMIGO-2 localized to other areas of the cancer cells not including the cell membrane of at least 2:1 indicates that the patient has an AMIGO-2-related cancer and is susceptible to AMIGO-2 therapy. In some embodiments a ratio of AMIGO-2 localized to the cell membrane compared to AMIGO-2 localized to other areas of the cancer cells not including the cell membrane of at least 3:1 indicates that the patient has an AMIGO-2-related cancer and is susceptible to AMIGO-2 therapy.

As used herein, the term “detecting” means to establish, discover, or ascertain evidence of an activity (for example, gene expression) or biomolecule (for example, a polypeptide).

A “native sequence” polypeptide is one that has the same amino acid sequence as a polypeptide derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “amino acid sequence variant” refers to polypeptides having amino acid sequences that differ to some extent from a native sequence polypeptide. Ordinarily, amino acid sequence variants will possess at least about 70%, at least about 80% homology or at least about 90% homology with at least one receptor binding domain of a native ligand or with at least one ligand binding domain of a native receptor or ligand binding domains thereof. The amino acid sequence variants possess substitutions, deletions, and/or insertions at certain positions within the amino acid sequence of the native amino acid sequence.

As used herein, the phrase “homologous nucleotide sequence,” or “homologous amino acid sequence,” or variations thereof, refers to sequences characterized by a homology, at the nucleotide level or amino acid level, of at least a specified percentage and is used interchangeably with “sequence identity”. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. Homologous amino acid sequences include those amino acid sequences which contain not more than 50 (e.g., not more than one, two, three, four, five, six, seven, eight, nine, ten, 12, 15, 20, 25, 30, 35, 40, or 50) conservative amino acid substitutions and which polypeptides retain at least 25% (e.g., at least 25%, at least 30%, at least 35%, at least 40%, at least 50%, at least 55%, at least 60%, at least 70%, at least 80%, at least 90%, at least 95%, at least 98%, at least 99%, at least 99.5%, or 100% or more) of the binding ability and/or activity of the native polypeptide. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Homologous amino acid sequences can include deletion variants of the amino acid sequences lacking one, two, three, four, five, six, seven, eight, nine, ten, 11, 12, 13, 14, 15, 16, 17, 18, 19, or 20 amino acid segments (of two or more amino acids) or non-contiguous single amino acids.

In some embodiments, the homologous amino acid sequences can contain insertions of one or more (e.g., one, two, three, four, five, six, seven, eight, nine, ten, 11, 12, 13, 14, 15, 20, more than 20) amino acids.

In some embodiments, the homologous amino acid sequences can contain conservative substitutions, insertions, and/or deletions.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, homology between the probe and target is between about 50% to about 60%. In some embodiments, nucleic acids have nucleotides that are about 70%, about 80%, about 85%, about 90%, about 92%, about 94%, about 95%, about 97%, about 98%, about 99% and about 100% homologous to SEQ ID NO:1, or a portion thereof. In some embodiments, homologs have the same activity as SEQ ID NO:1. In some embodiments, homologs share the same expression profile as SEQ ID NO:1.

Homology may also be at the polypeptide level. In some embodiments, polypeptides are about 60%, about 70%, about 80%, about 85%, about 90%, about 92%, about 94%, about 95%, about 97%, about 98%, about 99% and about 100% homologous to SEQ ID NO:2, or a portion thereof. In some embodiments, homologs have the same activity as SEQ ID NO:2. In some embodiments, homologs share the same expression profile as SEQ ID NO:2.

As used herein, the term “probe” refers to nucleic acid sequences of variable length. In some embodiments probes comprise at least about 10 and as many as about 6,000 nucleotides. In some embodiments probes comprise at least 12, at least 14, at least 16, at least 18, at least 20, at least 25, at least 50 or at least 75 consecutive nucleotides. Probes are used in the detection of identical; similar, or complementary nucleic acid sequences. Longer length probes are usually obtained from natural or recombinant sources, are highly specific to the target sequence, and are much slower to hybridize to the target than are oligomers. Probes may be single- or double-stranded and are designed to have specificity in PCR, hybridization membrane-based, in situ hybridization (ISH), fluorescent in situ hybridization (FISH), or ELISA-like technologies.

As used herein, the term “mixing” refers to the process of combining one or more compounds, cells, molecules, and the like together in the same area. This may be performed, for example, in a test tube, petri dish, or any container that allows the one or more compounds, cells, or molecules, to be mixed.

As used herein the term “isolated” refers to a polynucleotide, a polypeptide, an antibody, or a host cell that is in an environment different from that in which the polynucleotide, the polypeptide, or the antibody naturally occurs. Methods of isolating cells are well known to those skilled in the art. A polynucleotide, a polypeptide, or an antibody which is isolated is generally substantially purified.

As used herein, the term “substantially purified” refers to a compound (e.g., either a polynucleotide or a polypeptide or an antibody) that is removed from its natural environment and is at least 60% free, at least 75% free, and at least 90% free from other components with which it is naturally associated.

As used herein, the term “binding” means the physical or chemical interaction between two or more biomolecules or compounds. Binding includes ionic, non-ionic, hydrogen bonds, Van der Weals, hydrophobic interactions, etc. Binding can be either direct or indirect; indirect being through or due to the effects of another biomolecule or compound. Direct binding refers to interactions that do not take place through or due to the effect of another molecule or compound but instead are without other substantial chemical intermediates.

As used herein, the term “contacting” means bringing together, either directly or indirectly, one molecule into physical proximity to a second molecule. The molecule can be in any number of buffers, salts, solutions, etc. “Contacting” includes, for example, placing a polynucleotide into a beaker, microliter plate, cell culture flask, or a microarray, or the like, which contains a nucleic acid molecule. Contacting also includes, for example, placing an antibody into a beaker, microliter plate, cell culture flask, or microarray, or the like, which contains a polypeptide. Contacting may take place in vivo, ex vivo, or in vitro.

As used herein, the phrase “stringent hybridization conditions” or “stringent conditions” refers to conditions under which a probe, primer, or oligonucleotide will hybridize to its target sequence, but to a minimal number of other sequences. Stringent conditions are sequence-dependent and will be different in different circumstances. Longer sequences will hybridize with specificity to their proper complements at higher temperatures. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present in excess, at Tₘ 50% of the probes are hybridized to their complements at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 to 50 nucleotides) and at least about 60° C. for longer probes, primers or oligonucleotides. Stringent conditions may also be achieved with the addition of destabilizing agents, such as formamide.

As used herein, the term “moderate stringency conditions” refers to conditions under which a probe, primer, or oligonucleotide will hybridize to its target sequence, but to a limited number of other sequences. Moderate conditions are sequence-dependent and will be different in different circumstances. Moderate conditions are well-known to the art skilled and are described in, inter alia, Maniatis et al. (Molecular Cloning: A Laboratory Manual, Cold Spring Harbor Laboratory; 2nd Edition (December 1989)).

The nucleic acid compositions described herein can be used, for example, to produce polypeptides, as probes for the detection of mRNA in biological samples (e.g., extracts of human cells) or cDNA produced from such samples, to generate additional copies of the polynucleotides, to generate ribozymes or oligonucleotides (single and double stranded), and as single stranded DNA probes or as triple-strand forming oligonucleotides. The probes described herein can be used to, for example, determine the presence or absence of the polynucleotides provided herein in a sample. The polypeptides can be used to generate antibodies specific for a polypeptide associated with cancer, which antibodies are in turn useful in diagnostic methods, prognostic methods, and the like as discussed in more detail herein. Polypeptides are also useful as targets for therapeutic intervention, as discussed in more detail herein. Antibodies of the present invention may also be used, for example, to purify, detect, and target the polypeptides of the present invention, including both in vitro and in vivo diagnostic and therapeutic methods. For example, the antibodies are useful in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988). These and other uses are described in more detail below.

As used herein the term “imaging agent” refers to a composition linked to an antibody, small molecule, or probe of the invention that can be detected using techniques known to the art-skilled. As used herein, the term “evidence of gene expression” refers to any measurable indicia that a gene is expressed.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Specific examples of cancers that can be treated by the methods and compositions of the present invention include, but are not limited to, AMIGO-2 associated cancers. As used herein, “AMIGO-2 associated cancer” refers to a cancer characterized by cells that differentially express AMIGO-2 relative to non-cancerous cells. The present invention is also applicable to any tumor cell-type where AMIGO-2 plays a role in chromosomal stability, kinase activity, tumorigenicity, metastasis, signaling, cell adhesion, apoptosis, substrate phosphorylation, cell growth, tumor formation, cyclin production, cell proliferation, cell cycle regulation, cancer cell growth, cancer cell survival, anchorage-independent growth, and angiogenesis, cell migration, cell-cell interaction, among others.

In some embodiments, the cancer is lung, bladder, kidney, colon, breast, uterine, ovarian, or pancreatic cancer, or a cancer metastasis. In some embodiments, the cancer is lung or colon cancer. In some embodiments, the cancer is a cancer other than gastric cancer. In some embodiments, such cancers exhibit differential expression of AMIGO-2 of at least about 25%, at least about 50%, at least about 75%, at least about 100%, at least about 150%, at least about 200%, or at least about 300% as compared to a control.

The present invention provides methods and compositions that provide for the treatment, inhibition, and management of diseases and disorders associated with AMIGO-2 overexpression as well as the treatment, inhibition, and management of symptoms of such diseases and disorders. Some embodiments of the invention relate to methods and compositions comprising compositions that treat, inhibit or manage cancer including, without limitation, cancer metastases, cancer cell survival, cancer cell proliferation, cancer cell growth, cell cycle regulation, angiogenesis and cancer cell invasiveness.

The present invention further provides methods including other active ingredients in combination with the AMIGO-2 modulators of the present invention. In some embodiments, the methods further comprise administering one or more conventional cancer therapeutics to the patient. In some embodiments the methods of the present invention further comprise treating the patient with one or more of chemotherapy, radiation therapy or surgery.

The present invention also provides methods and compositions for the treatment, inhibition, and management of cancer or other hyperproliferative cell disorder or disease that has become partially or completely refractory to current or standard cancer treatment, such as surgery, chemotherapy, radiation therapy, hormonal therapy, and biological therapy.

The invention also provides diagnostic and/or imaging methods using the AMIGO-2 modulators of the invention, particularly AMIGO-2 antibodies, to diagnose cancer and/or predict cancer progression. In some embodiments, the methods of the invention provide methods of imaging and localizing tumors and/or metastases and methods of diagnosis and prognosis. In some embodiments, the methods of the invention provide methods to evaluate the appropriateness and/or effectiveness of AMIGO-2-related therapy.

AMIGO-2 Modulators

The present invention provides AMIGO-2 modulators for, inter alias, the treatment, diagnosis, detection or imaging of cancer. AMIGO-2 modulators are also useful in the preparation of medicaments for the treatment of cancer. In some embodiments the AMIGO-2 modulator is an AMIGO-2 inhibitor.

In some embodiments, the AMIGO-2 modulator is a nucleotide, a small molecule, a mimetic, a decoy, or an antibody. In some embodiments the AMIGO-2 modulator is an isolated double-stranded RNA (dsRNA); an isolated oligonucleotide comprising at least 10 consecutive nucleotides of a sequence selected from the group consisting of SEQ ID NOs:7-16; an antibody that binds an epitope in a domain of AMIGO-2 selected from the group consisting of the signal peptide, the LRRCT domain, LRR1 domain, LRR2 domain, LRR3 domain, LRR4 domain, LRR5 domain, LRR6 domain, LRRCT domain, Ig V-set domain, and Ig domain; a small molecule; a mimetic; a soluble receptor; or a decoy. In some embodiments, an antibody that binds an epitope in a domain of AMIGO-2 does not specifically bind to a polypeptide other than AMIGO-2. For example, in some embodiments, the antibody fragment does not specifically bind to AMIGO-1 or AMIGO-3.

In some embodiments, the AMIGO-2 modulator inhibits an AMIGO-2 activity by at least 25%, 50%, 75%, 80%, 90%, 95%, 97%, 98%, 99% or 100%, as compared to a control. In some embodiments, the AMIGO-2 modulator inhibits LIV-1 expression by at least 25%, 50%, 75%, 80%, 90%, 95%, 97%, 98%, 99% or 100%, as compared to a control.

Antibodies

In some embodiments the AMIGO-2 modulator is a monoclonal antibody, a polyclonal antibody, a chimeric antibody, a human antibody, a humanized antibody, a single-chain antibody, or a Fab fragment. The antibody or Fab fragment may be labeled with, for example, an enzyme, radioisotope, or fluorophore. In some embodiments, the antibody or Fab fragment does not specifically bind to a polypeptide other than AMIGO-2. For example, in some embodiments, the antibody or Fab fragment does not specifically bind to AMIGO-1 or AMIGO-3. In some embodiments the antibody or Fab fragment has a binding affinity less than about 1×10⁵ Ka for a polypeptide other than AMIGO-2. In some embodiments, the AMIGO-2 modulator is a monoclonal antibody which binds to AMIGO-2 with an affinity of at least 1×10⁸ Ka.

The invention also provides antibodies that competitively inhibit binding of an antibody to an epitope of the invention as determined by any method known in the art for determining competitive binding using, for example, immunoassays. In some embodiments, the antibody competitively inhibits binding to the epitope by at least 95%, at least 90%, at least 85%, at least 80%, at least 75%, at least 70%, at least 60%, or at least 50%.

In some embodiments the antibody is a humanized antibody. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

Antibodies of the present invention may function through different mechanisms. In some embodiments, antibodies trigger antibody-dependent cellular cytotoxicity (ADCC), a lytic attack on antibody-targeted cells. In some embodiments, antibodies have multiple therapeutic functions, including, for example, antigen-binding, induction of apoptosis, and complement-dependent cellular cytotoxicity (CDC). In some embodiments, the antibody is conjugated to a toxin or radionuclide.

In some embodiments, antibodies of the present invention may act as AMIGO-2 antagonists. For example, in some embodiments the present invention provides antibodies which disrupt the receptor/ligand interactions with the polypeptides of the invention either partially or fully. In some embodiments antibodies of the present invention bind an epitope disclosed herein, or a portion thereof. In some embodiments, antibodies are provided that modulate ligand activity or receptor activity by at least 95%, at least 90%, at least 85%, at least 80%, at least 75%, at least 70%, at least 60%, or at least 50% compared to the activity in the absence of the antibody.

In some embodiments the present invention provides neutralizing antibodies. A neutralizing antibody binds an infectious agent, such as a virus or a bacterium, such as a virus or bacterium associated with cancer (e.g., a JC polyoma virus, Epstein-Barr virus, or Helicobacter pylori). In some embodiments the neutralizing antibodies can effectively act as receptor antagonists, i.e., inhibiting either all or a subset of the biological activities of the ligand-mediated receptor activation. In some embodiments the antibodies may be specified as agonists, antagonists or inverse agonists for biological activities comprising the specific biological activities of the peptides of the invention disclosed herein.

In some embodiments the antibodies inhibit one or more AMIGO-2 activities selected from the group consisting of chromosomal stability, kinase activity, tumorigenicity, metastasis, signaling, cell adhesion, cell apoptosis, substrate phosphorylation, cancer cell growth, tumor formation, cyclin production, cell proliferation, progression through the cell cycle (e.g., progression into the G2/M stage of the cell cycle), anchorage-independent growth, localization of AMIGO-2 protein to the cell membrane, interactions between AMIGO-2 and one or both of AMIGO-1 or AMIGO-3, and angiogenesis, among others.

In some embodiments, AMIGO-2 antibodies inhibit growth and survival pathways, such as those mediated by activated EGFR and mutated beta-catenin. In some embodiments AMIGO-2 antibodies inhibit (e.g., inhibit mRNA or protein expression) one or more of cyclin D1, cyclin B1, c-Myc, c-Jun, FosL1, Extracellular signal-Regulated Kinase (ERK); Vascular Endothelial Growth Factor (VEGF), urokinase, and Poly(ADP-Ribose)Polymerase 1 (PARP1). Regulation of VEGF and urokinase also indicate a role for Amigo-2 in angiogenesis. In some embodiments AMIGO-2 antibodies modulate cancer cell motility and affect cancer metastasis.

In some embodiments, AMIGO-2 antibodies regulate immediate early genes and pathways comprising immediate early genes. For example, the AMIGO-2 antibodies regulate one of more of cFosL1, E2F1, ELK1, JNK, MEKK, SAPK1 p38, cyclin D, c-Jun, c-fos, c-myc, JE, KC, junB, and BTG2 and related pathways.

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

In addition to chimeric and humanized antibodies, fully human antibodies can be derived from transgenic mice having human immunoglobulin genes (see, e.g., U.S. Pat. Nos. 6,075,181, 6,091,001, and 6,114,598, all of which are incorporated herein by reference), or from phage display libraries of human immunoglobulin genes (see, e.g. McCafferty et al., Nature, 348:552-554 (1990). Clackson et al., Nature, 352:624-628 (1991), and Marks et al., J. Mol. Biol., 222:581-597 (1991)). In some embodiments, antibodies may be produced and identified by scFv-phage display libraries. Antibody phage display technology is available from commercial sources such as from Morphosys.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, in some embodiments in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas for expression, antibodies can be produced in a cell line such as a CHO or myeloma cell line, as disclosed in U.S. Pat. Nos. 5,545,403; 5,545,405; and 5,998,144; each incorporated herein by reference. Briefly the cell line is transfected with vectors capable of expressing a light chain and a heavy chain, respectively. By transfecting the two proteins on separate vectors, chimeric antibodies can be produced. Immunol. 147:8; Banchereau et al. (1991) Clin. Immunol. Spectrum 3:8; and Banchereau et al. (1991) Science 251:70; all of which are herein incorporated by reference.

Human antibodies can also be produced using techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86 95 (1991)). Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779 783 (1992); Lonberg et al., Nature 368 856 859 (1994); Morrison, Nature 368, 812 13 (1994); Fishwild et al., Nature Biotechnology 14, 845 51 (1996); Neuberger, Nature Biotechnology 14, 826 (1996); Lonberg and Huszar, Intern. Rev. Immunol. 13 65 93 (1995); Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference. Fully humanized antibodies can be identified in screening assays using commercial resources such as Morphosys (Martinsried/Planegg, Germany).

Human antibodies can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions; and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Antibodies of the present invention can also be produced using human engineering techniques as discussed in U.S. Pat. No. 5,766,886, which is incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

Antibodies of the present invention may be administered to a subject via in vivo therapeutic antibody gene transfer as discussed by Fang et al. (2005), Nat. Biotechnol. 23, 584-590. For example recombinant vectors can be generated to deliver a multicistronic expression cassette comprising a peptide that mediates enzyme independent, cotranslational self cleavage of polypeptides placed between MAb heavy and light chain encoding sequences. Expression leads to stoichiometric amounts of both MAb chains. In some embodiments the peptide that mediates enzyme independent, cotranslational self cleavage is the foot-and-mouth-disease derived 2A peptide.

Fragments of antibodies are suitable for use in the methods of the invention so long as they retain the desired affinity of the full-length antibody. Thus, a fragment of an anti-AMIGO-2 antibody will retain an ability to bind to AMIGO-2. Such fragments are characterized by properties similar to the corresponding full-length anti-AMIGO-2 antibody, that is, the fragments will specifically bind a human AMIGO-2 antigen expressed on the surface of a human cell.

In some embodiments, the antibodies specifically bind to one or more epitopes in an extracellular domain of AMIGO-2. In some embodiments, the antibodies modulate one or more AMIGO-2 related biological activities. In some embodiments the antibodies inhibit one or more of kinase activity, tumorigenicity, metastasis, AMIGO-2 signaling, AMIGO-2 mediated cell adhesion, cancer cell apoptosis, ERK phosphorylation cell growth, tumor formation, cyclin production, cell proliferation, progression through the cell cycle, anchorage-independent growth, localization of AMIGO-2 protein to the cell membrane, interactions between AMIGO-2 and one or both of AMIGO-1 or AMIGO-3, and angiogenesis, among others.

In some embodiments the antibody is a monoclonal antibody or Fab fragment which specifically binds to one or more AMIGO-2 epitopes in a domain selected from the group consisting of the signal peptide domain, the LRRNT domain, LRR1 domain, LRR2 domain, LRR3 domain, LRR4 domain, LRR5 domain, LRR6 domain, LRRCT domain, Ig V-set domain, and Ig domain of AMIGO-2. The signal peptide domain is from about amino acids 1-33 of SEQ ED NO:2; the LRR-N-terminal (LRR-NT) domain is from about amino acids 41-60 of SEQ ID NO:2; the LRR1 domain is from about amino acids 69-92 of SEQ ID NO:2; the LRR2 domain is from about amino acids 94-116; the LRR3 domain is from about amino acids 118-140; the LRR4 domain is from about amino acids 142-164; the LRR5 domain is from about 167-191; the LRR6 domain is from about amino acids 193-216; the LRR-C-terminal (LRR-CT) domain is from about amino acids 222-282; the Ig V-set domain is from about 293-388; and the Ig domain of AMIGO-2 is within the V-set domain, from about amino acids 303-365. In some embodiments the antibody is a monoclonal antibody that specifically binds to one or more epitopes in the signal peptide of AMIGO-2. In one embodiment, the antibody does not bind specifically to an epitope that is not an AMIGO-2 epitope. For example, in one embodiment, the antibody does not bind specifically to an AMIGO-1 epitope or an AMIGO-3 epitope.

In some embodiments, the AMIGO-2 modulator is a monoclonal antibody or Fab fragment which specifically hinds to one or more epitopes of the LRRNT domain of AMIGO-2.

In some embodiments, the AMIGO-2 modulator is a monoclonal antibody or Fab fragment which specifically binds to one or more epitopes of the LRR1 domain of AMIGO-2. In some embodiments the LRR1 epitope is selected from the group consisting of SEQ ID NO:30 and SEQ ID NO:57.

In some embodiments, the AMIGO-2 modulator is a monoclonal antibody or Fab fragment which specifically binds to one or more epitopes of the LRR2 domain of AMIGO-2. In some embodiments the LRR2 epitope is selected from the group consisting of SEQ ID NO:32, SEQ ED NO:39, and SEQ ID NO:61.

In some embodiments, the AMIGO-2 modulator is a monoclonal antibody or Fab fragment which specifically binds to one or more epitopes of the LRR3 domain of AMIGO-2. In some embodiments the LRR3 epitope is selected from the group consisting of SEQ ID NO:29, SEQ ID NO:39, SEQ ID NO:40, SEQ ID NO:56 and SEQ ID NO:58.

In some embodiments, the AMIGO-2 modulator is a monoclonal antibody or Fab fragment which specifically binds to one or more epitopes of the LRR4 domain of AMIGO-2. In some embodiments said the LRR4 epitope is selected from the group consisting of SEQ ID NO:26, SEQ ID NO:35, and SEQ ID NO:45.

In some embodiments, the AMIGO-2 modulator is a monoclonal antibody or Fab fragment which specifically binds to one or more epitopes of the LRR5 domain of AMIGO-2. In some embodiments the LRR5 epitope is selected from the group consisting of SEQ ID NO:25, SEQ ID NO:27, SEQ ID NO:36, and SEQ ID NO:53.

In some embodiments, the AMIGO-2 modulator is a monoclonal antibody or Fab fragment which specifically binds to one or more epitopes of the LRR6 domain of AMIGO-2. In some embodiments the LRR6 epitope is selected from the group consisting of SEQ ID NO:31, SEQ ID NO:33, SEQ ID NO:41, SEQ ID NO:46, SEQ ID NO:47, and SEQ ID NO:62.

In some embodiments, the AMIGO-2 modulator is a monoclonal antibody or Fab fragment which specifically binds to one or more epitopes of the LRRCT domain of AMIGO-2. In some embodiments the LRRCT epitope is selected from the group consisting of SEQ ID NO:28, SEQ ID NO:34, SEQ ID NO:37, SEQ ID NO:38, SEQ ID NO:44, SEQ ID NO:49, SEQ ID NO:50, SEQ ID NO:51, SEQ ID NO:52, SEQ ID NO:54, SEQ ID NO:59, SEQ ID NO:60, and SEQ ID NO:62.

In some embodiments, the AMIGO-2 modulator is a monoclonal antibody or Fab fragment which specifically binds to one or more epitopes of the Ig V-set domain of AMIGO-2.

In some embodiments, the AMIGO-2 modulator is a monoclonal antibody or Fab fragment which specifically binds to one or more epitopes of the Ig domain of AMIGO-2.

In some embodiments, the AMIGO-2 modulator is a monoclonal antibody or Fab fragment which specifically binds to one or more epitopes in the extracellular domain (ECD) of AMIGO-2. In some embodiments, the AMIGO-2 modulator is a monoclonal antibody or Fab fragment which specifically binds to one or more epitopes in a sequence consisting essentially of SEQ ID NO:3.

Suitable antibodies according to the present invention can recognize linear or conformational epitopes, or combinations thereof. In some embodiments the antibodies of the present invention bind to epitopes of antigenic regions of AMIGO-2 selected from the group consisting of SEQ ID NOs:3-6 and 25-62. In some embodiments, the AMIGO-2 modulator is a monoclonal antibody or Fab fragment which specifically binds to one or more epitopes in a sequence consisting essentially of SEQ ID NO:3. In some embodiments the antibody is specific for an epitope having a sequence selected from the group consisting of SEQ ID NO:3.

It is to be understood that these peptides may not necessarily precisely map one epitope, but may also contain an AMIGO-2 sequence that is not immunogenic.

Methods of predicting other potential epitopes to which an antibody of the invention can bind are well-known to those of skill in the art and include without limitation, Kyte-Doolittle Analysis (Kyte, J. and Dolittle, R. F., J. Mol. Biol. (1982) 157:105-132), Hopp and Woods Analysis (Hopp, T. P. and Woods, K. R., Proc. Natl. Acad. Sci. USA (1981) 78:3824-3828; Hopp, T. J. and Woods, K. R., Mol. Immunol. (1983) 20:483-489; Hopp, T. J., J. Immunol. Methods (1986) 88:1-18.), Jameson-Wolf Analysis (Jameson, B. A. and Wolf, H., Comput. Appl. Biosci. (1988) 4:181-186.), and Emini Analysis (Emini, E. A., Schlief, W. A., Colonno, R. J. and Wimmer, E., Virology (1985) 140:13-20.). In some embodiments, potential epitopes are identified by determining theoretical extracellular domains. Analysis algorithms such as TMpred (see K. Hofmann & W. Stoffel (1993) TMbase—A database of membrane spanning proteins segments Biol. Chem. Hoppe-Seyler 374,166) or TMHMM (A. Krogh, B. Larsson, G. von Heijne, and E. L. L. Sonnhammer. Predicting transmembrane protein topology with a hidden Markov model: Application to complete genomes. Journal of Molecular Biology, 305(3):567-580, January 2001) can be used to make such preditions. Other algorithms, such as SignalP 3.0 (Bednsten et al, (2004) J Mol Biol. 2004 Jul. 16; 340(4):783-95) can be used to predict the presence of signal peptides and to predict where those peptides would be cleaved from the full-length protein. The portions of the proteins on the outside of the cell can serve as targets for antibody interaction.

Antibodies are defined to be “specifically binding” if: 1) they exhibit a threshold level of binding activity, and/or 2) they do not significantly cross-react with known related polypeptide molecules. The binding affinity of an antibody can be readily determined by one of ordinary skill in the art, for example, by Scatchard analysis (Scatchard, Ann. NY Acad. Sci. 51: 660-672, 1949). In some embodiments the antibodies of the present invention bind to their target epitopes or mimetic decoys at least 1.5-fold, 2-fold, 5-fold 10-fold, 100-fold, 10³-fold, 10⁴-fold, 10⁵-fold, 10⁶-fold or greater for the target cancer-associated polypeptide than to other known members of the AMIGO family (e.g. AMIGO-1 and AMIGO-3).

In some embodiments the antibodies bind with high affinity of 10⁻⁴M or less, 10⁻⁷M or less, 10⁻⁹ M or less or with subnanomolar affinity (0.9, 0.8, 0.7, 0.6, 0.5, 0.4, 0.3, 0.2, 0.1 nM or even less). In some embodiments the binding affinity of the antibodies for AMIGO-2 is at least 1×10⁶ Ka. In some embodiments the binding affinity of the antibodies for AMIGO-2 is at least 5×10⁶ Ka, at least 1×10⁷ Ka, at least 2×10⁷ Ka, at least 1×10⁸ Ka, or greater. Antibodies of the present invention may also be described or specified in terms of their binding affinity to a polypeptide of the invention. In some embodiments binding affinities include those with a K_{d} less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶M, 10⁻⁶M, 5×10⁻⁷ M, 10⁻⁷M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies of the present invention do not bind to known related polypeptide molecules, for example, if they bind AMIGO-2 polypeptide but not known related polypeptides using a standard Western blot analysis (Ausubel et al., Current Protocols in Molecular Biology, 1994). Examples of known related polypeptides include, without limitation, other members of the AMIGO family (e.g., AMIGO-1 and AMIGO-3).

In some embodiments, the antibodies of the present invention bind to orthologs, homologs, paralogs or variants, or combinations and subcombinations thereof, of AMIGO-2. In some embodiments, the antibodies of the present invention bind to orthologs of AMIGO-2, In some embodiments, the antibodies of the present invention bind to homologs of AMIGO-2. Homologs of AMIGO-2 refer to the known AMIGO-2-related proteins, including AMIGO-1 and AMIGO-3. In some embodiments, the antibodies of the present invention bind to paralogs of AMIGO-2. In some embodiments, the antibodies of the present invention bind to variants of AMIGO-2. In some embodiments, the antibodies of the present invention do not bind to orthologs, homologs, paralogs or variants, or combinations and subcombinations thereof of AMIGO-2.

In some embodiments, antibodies may be screened against known related polypeptides to isolate an antibody population that specifically binds to AMIGO-2 polypeptides. For example, antibodies specific to human AMIGO-2 polypeptides will flow through a column comprising AMIGO proteins (with the exception of AMIGO-2) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (ads.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

In some embodiments, the antibodies of the present invention do not specifically bind to epitopes within the sequences selected from the group consisting of SEQ ID NO:63 (AMIGO-1) and SEQ ID NO:64 (AMIGO-3). In some embodiments the antibodies or Fab fragments do not cross-react with AMIGO-1 or AMIGO-3.

The invention also provides antibodies that are SMIPs or binding domain immunoglobulin fusion proteins specific for a target protein. These constructs are single-chain polypeptides comprising antigen binding domains fused to immunoglobulin domains necessary to carry out antibody effector functions. See e.g., WO03/041600, U.S. Patent Publication 20030133939 and US Patent Publication 20030118592.

In some embodiments the antibodies of the present invention are neutralizing antibodies. In some embodiments the antibodies are targeting antibodies. In some embodiments, the antibodies are internalized upon binding a target. In some embodiments the antibodies do not become internalized upon binding a target and instead remain on the surface.

In some embodiments, the neutralizing antibody will not have any effector functions. Alternatively, a neutralizing antibody can have effector functions.

The antibodies of the present invention can be screened for the ability to either be rapidly internalized upon binding to the tumor-cell antigen in question, or for the ability to remain on the cell surface following binding. In some embodiments, for example in the construction of some types of immunoconjugates, the ability of an antibody to be internalized may be desired if internalization is required to release the toxin moiety. Alternatively, if the antibody is being used to promote ADCC or CDC, it may be more desirable for the antibody to remain on the cell surface. A screening method can be used to differentiate these types of behaviors. For example, a tumor cell antigen hearing cell may be used where the cells are incubated with human IgG1 (control antibody) or one of the antibodies of the invention at a concentration of approximately 1 μg/mL on ice (with 0.1% sodium azide to block internalization) or 37° C. (without sodium azide) for 3 hours. The cells are then washed with cold staining buffer (PBS+1% BSA+0.1% sodium azide), and are stained with goat anti-human IgG-FITC for 30 minutes on ice. Geometric mean fluorescent intensity (MFI) is recorded by FACS Calibur. If no difference in MFI is observed between cells incubated with the antibody of the invention on ice in the presence of sodium azide and cells observed at 37° C. in the absence of sodium azide, the antibody will be suspected to be one that remains bound to the cell surface, rather than being internalized. If however, a decrease in surface stainable antibody is found when the cells are incubated at 37° C. in the absence of sodium azide, the antibody will be suspected to be one which is capable of internalization.

Antibody Conjugates

In some embodiments, the antibodies of the invention are conjugated. In some embodiments, the conjugated antibodies are useful for cancer therapeutics, cancer diagnosis, or imaging of cancerous cells.

For diagnostic applications, the antibody typically will be labeled with a detectable moiety. Numerous labels are available which can be generally grouped into the following categories:

(a) Radionuclides such as those discussed infra. The antibody can be labeled, for example, with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

The antibodies may also be used for in vivo diagnostic assays. In some embodiments, the antibody is labeled with a radionuclide so that the tumor can be localized using immunoscintiography. As a matter of convenience, the antibodies of the present invention can be provided in a kit, i.e., a packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In some embodiments, antibodies are conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate. In some embodiments, the conjugate may be the highly potent maytansine derivative DM1 (N2′-deacetyl-N2′-(3-mercapto-1-oxopropyl)-maytansine) (see for example WO02/098883 published Dec. 12, 2002) which has an IC50 of approximately 10-11 M (review, see Payne (2003) Cancer Cell 3:207-212) or DM4 (N2′-deacetyl-N2′(4-methyl-4-mercapto-1-oxopentyl)-maytansine) (see, for example, WO2004/103272 published Dec. 2, 2004).

In some embodiments the antibody conjugate comprises an anti-tumor cell antigen antibody conjugated to one or more calicheamicin molecules. The calicheamicin family of antibiotics is capable of producing double-stranded DNA breaks at sub-picomolar concentrations. Structural analogues of calicheamicin which may be used include, but are not limited to, gammal1, alpha2I, alpha3I, N-acetyl-gammal1, PSAG and thetaI1 (Hinman et al. Cancer Research 53: 3336-3342 (1993) and Lode et al. Cancer Research 58: 2925-2928 (1998)). See, also, U.S. Pat. Nos. 5,714,586; 5,712,374; 5,264,586; and 5,773,001, each of which is expressly incorporated herein by reference.

In some embodiments the antibody is conjugated to a prodrug capable of being released in its active form by enzymes overproduced in many cancers. For example, antibody conjugates can be made with a prodrug form of doxorubicin wherein the active component is released from the conjugate by plasmin. Plasmin is known to be over produced in many cancerous tissues (see Decy et al, (2004) FASEB Journal 18(3): 565-567).

In some embodiments the antibodies are conjugated to enzymatically active toxins and fragments thereof. In some embodiments the toxins include, without limitation, diphtheria A chain, nonbinding active fragments of diphtheria toxin, exotoxin A chain (from Pseudomonas aeruginosa), Pseudomonas endotoxin, ricin A chain, abrin A chain, modeccin A chain, alpha-sarcin, Aleurites fordii proteins, dianthin proteins, Phytolaca americana proteins (PAPI, PAPII, and PAP-S), Ribonuclease (Rnase), Deoxyribonuclease (Dnase), pokeweed antiviral protein, momordica charantia inhibitor, curcin, crotin, sapaonaria officinalis inhibitor, gelonin, mitogellin, restrictocin, phenomycin, neomycin and the tricothecenes. See, for example, WO 93/21232 published Oct. 28, 1993. In some embodiments the toxins have low intrinsic immunogenicity and a mechanism of action (e.g. a cytotoxic mechanism versus a cytostatic mechanism) that reduces the opportunity for the cancerous cells to become resistant to the toxin.

In some embodiments conjugates are made between the antibodies of the invention and immunomodulators. For example, in some embodiments immunostimulatory oligonucleotides can be used. These molecules are potent immunogens that can elicit antigen-specific antibody responses (see Datta et al, (2003) Ann N.Y. Acad. Sci 1002: 105-111). Additional immunomodulatory compounds can include stem cell growth factor such as “S1 factor”, lymphotoxins such as tumor necrosis factor (TNF), hematopoietic factor such as an interleukin, colony stimulating factor (CSF) such as granulocyte-colony stimulating factor (G-CSF) or granulocyte macrophage-stimulating factor (GM-CSF), interferon (IFN) such as interferon alpha, beta or gamma, erythropoietin, and thrombopoietin.

In some embodiments radioconjugated antibodies are provided. In some embodiments such antibodies can be made using ³²P, ³³P, ⁴⁷Sc, ⁵⁹Fe, ⁶⁴Cu, ⁶⁷Cu, ⁷⁵Se, ⁷⁷As, ⁸⁹Sr, ⁹⁰Y, ⁹⁹Mo, ¹⁰⁵Rh, ¹⁰⁹Pd, ¹²⁵I, ¹³¹I, ¹⁴²Pr, ¹⁴³Pr, ¹⁴⁹Pm, ¹⁵³Sm, ¹⁶¹Th, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹Pb, ²¹²Pb, ²¹³Bi, ⁵⁸Co, ⁶⁷Ga, ^{80m}Br, ^{99m}Tc, ^{103m}Rh, ¹⁰⁹Pt, ¹⁶¹Ho, ^{189m}Os, ¹⁹²Ir, ¹⁵²Dy, ²¹¹At, ²¹²Bi, ²²³Ra, ²¹⁹Rn, ²¹⁵Po, ²¹¹Bi, ²²⁵Ac, ²²¹Fr, ²¹⁷At, ²¹³Bi, ²⁵⁵Fm and combinations and subcombinations thereof. In some embodiments, boron, gadolinium or uranium atoms are conjugated to the antibodies. In some embodiments the boron atom is ¹⁰B, the gadolinium atom is ¹⁵⁷Gd, and the uranium atom is ²³⁵U.

In some embodiments the radionuclide conjugate has a radionuclide with an energy between 20 and 10,000 keV. The radionuclide can be an Auger emitter, with an energy of less than 1000 keV, a P emitter with an energy between 20 and 5000 keV, or an alpha or ‘a’ emitter with an energy between 2000 and 10,000 keV.

In some embodiments diagnostic radioconjugates are provided which comprise a radionuclide that is a gamma-, beta-, or positron-emitting isotope. In some embodiments the radionuclide has an energy between 20 and 10,000 keV. In some embodiments the radionuclide is selected from the group of ¹⁸F, ⁵¹Mn, ^{52m}Mn, ⁵²Fe, ⁵⁵Co, ⁶²Cu, ⁶⁴Cu, ⁶⁸Ga, ⁷²As, ⁷⁵Br, ⁷⁶Br, ^{82m}Rb, ⁸³Sr, ⁸⁹Zr, ^{94m}Tc, ⁵¹Cr, ⁵⁷Co, ⁵⁸Co, ⁵⁹Fe, ⁶⁷Ga, ⁷⁵Se, ⁹⁷Ru, ^{99m}Tc, ^{114m}In, ¹²³I, ¹²⁵I, ¹³Li and ¹⁹⁷Hg.

In some embodiments the antibodies of the invention are conjugated to diagnostic agents that are photoactive or contrast agents. Photoactive compounds can comprise compounds such as chromagens or dyes. Contrast agents may be, for example a paramagnetic ion, wherein the ion comprises a metal selected from the group of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). The contrast agent may also be a radio-opaque compound used in X-ray techniques or computed tomography, such as an iodine, iridium, barium, gallium and thallium compound. Radioopaque compounds may be selected from the group of barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride. In some embodiments, the diagnostic immunoconjugates may contain ultrasound-enhancing agents such as a gas filled liposome that is conjugated to an antibody of the invention. Diagnostic immunoconjugates may be used for a variety of procedures including, but not limited to, intraoperative, endoscopic or intravascular methods of tumor or cancer diagnosis and detection.

In some embodiments antibody conjugates are made using a variety of bifunctional protein coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used. Agents may additionally be linked to the antibodies of the invention through a carbohydrate moiety.

In some embodiments fusion proteins comprising the antibodies of the invention and cytotoxic agents may be made, e.g. by recombinant techniques or peptide synthesis. In some embodiments such immunoconjugates comprising the anti-tumor antigen antibody conjugated with a cytotoxic agent are administered to the patient. In some embodiments the immunoconjugate and/or tumor cell antigen protein to which it is bound is/are internalized by the cell, resulting in increased therapeutic efficacy of the immunoconjugate in killing the cancer cell to which it binds. In some embodiments, the cytotoxic agent targets or interferes with nucleic acid in the cancer cell. Examples of such cytotoxic agents include maytansinoids, calicheamicins, ribonucleases and DNA endonucleases.

In some embodiments the antibodies are conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

In some embodiments the antibodies are conjugated to a cytotoxic molecule which is released inside a target cell lysozome. For example, the drug monomethyl auristatin E (MMAE) can be conjugated via a valine-citrulline linkage which will be cleaved by the proteolytic lysozomal enzyme cathepsin B following internalization of the antibody conjugate (see for example WO03/026577 published Apr. 3, 2003). In some embodiments, the MMAE can be attached to the antibody using an acid-labile linker containing a hydrazone functionality as the cleavable moiety (see for example WO02/088172 published Nov. 11, 2002).

Antibody Dependent Enzyme Mediated Prodrug Therapy (ADEPT)

In some embodiments the antibodies of the present invention may be used in ADEPT by conjugating the antibody to a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

In some embodiments the enzyme component of the immunoconjugate useful for ADEPT includes any enzyme capable of acting on a prodrug in such a way so as to convert it into its more active, cytotoxic form.

Enzymes that are useful in ADEPT include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. In some embodiments antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

In some embodiments the ADEPT enzymes can be covalently, bound to the antibodies by techniques well known in the art such as the use of the heterobifunctional crosslinking reagents discussed above. In some embodiments, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

In some embodiments identification of an antibody that acts in a cytostatic manner rather than a cytotoxic manner can be accomplished by measuring viability of a treated target cell culture in comparison with a non-treated control culture. Viability can be detected using methods known in the art such as the CellTiter-Blue® Cell Viability Assay or the CellTiter-Glo® Luminescent Cell Viability Assay (Promega, catalog numbers G8080 and G5750 respectively). In some embodiments an antibody is considered as potentially cytostatic if treatment causes a decrease in cell number in comparison to the control culture without any evidence of cell death as measured by the means described above.

In some embodiments an in vitro screening assay can be performed to identify an antibody that promotes ADCC using assays known in the art. One exemplary assay is the In Vitro ADCC Assay. To prepare chromium 51-labeled target cells, tumor cell lines are grown in tissue culture plates and harvested using sterile 10 mM EDTA in PBS. The detached cells are washed twice with cell culture medium. Cells (5×10⁶) are labeled with 200 μCi of chromium 51 (New England Nuclear/DuPont) at 37° C. for one hour with occasional mixing. Labeled cells are washed three times with cell culture medium, then are resuspended to a concentration of 1×10⁵ cells/mL. Cells are used either without opsonization, or are opsonized prior to the assay by incubation with test antibody at 100 ng/mL and 1.25 ng/mL in PBMC assay or 20 ng/mL and 1 ng/mL in NK assay. Peripheral blood mononuclear cells are prepared by collecting blood on heparin from normal healthy donors and diluted with an equal volume of phosphate buffered saline (PBS). The blood is then layered over LYMPHOCYTE SEPARATION MEDIUM® (LSM: Organon Teknika) and centrifuged according to the manufacturer's instructions. Mononuclear cells are collected from the LSM-plasma interface and are washed three times with PBS. Effector cells are suspended in cell culture medium to a final concentration of 1×10⁷ cells/mL. After purification through LSM, natural killer (NK) cells are isolated from PBMCs by negative selection using an NK cell isolation kit and a magnetic column (Miltenyi Biotech) according to the manufacturer's instructions. Isolated NK cells are collected, washed and resuspended in cell culture medium to a concentration of 2×10⁶ cells/mL. The identity of the NK cells is confirmed by flow cytometric analysis. Varying effector:target ratios are prepared by serially diluting the effector (either PBMC or NK) cells two-fold along the rows of a microliter plate (100 μL final volume) in cell culture medium. The concentration of effector cells ranges from 1.0×10⁷/mL to 2.0×10⁴/mL for PBMC and from 2.0×10⁶/mL to 3.9×10³/mL for NK. After titration of effector cells, 100 μL of chromium 51-labeled target cells (opsonized or nonoponsonized) at 1×10⁵ cells/mL are added to each well of the plate. This results in an initial effector:target ratio of 100:1 for PBMC and 20:1 for NK cells. All assays are run in duplicate, and each plate contains controls for both spontaneous lysis (no effector cells) and total lysis (target cells plus 100 μL 1% sodium dodecyl sulfate, 1 N sodium hydroxide). The plates are incubated at 37° C. for 18 hours, after which the cell culture supernatants are harvested using a supernatant collection system (Skatron Instrument, Inc.) and counted in a Minaxi auto-gamma 5000 series gamma counter (Packard) for one minute. Results are then expressed as percent cytotoxicity using the formula: % Cytotoxicity=(sample cpm-spontaneous lysis)/(total lysis-spontaneous lysis)×100.

To identify an antibody that promotes CDC, the skilled artisan may perform an assay known in the art. One exemplary assay is the In Vitro CDC assay. In vitro, CDC activity can be measured by incubating tumor cell antigen expressing cells with human (or alternate source) complement-containing serum in the absence or presence of different concentrations of test antibody. Cytotoxicity is then measured by quantifying live cells using ALAMAR BLUE® (Gazzano-Santoro et al., J. Immunol. Methods 202 163-171 (1997)). Control assays are performed without antibody, and with antibody, but using heat inactivated serum and/or using cells which do not express the tumor cell antigen in question. Alternatively, red blood cells can be coated with tumor antigen or peptides derived from tumor antigen, and then CDC may be assayed by observing red cell lysis (see for example Karjalainen and Mantyjarvi, Acta Pathol Microbiol Scand [C]. 1981 October; 89(5):315-9).

To select for antibodies that induce cell death, loss of membrane integrity as indicated by, e.g., PI, trypan blue or 7AAD uptake may be assessed relative to control. One exemplary assay is the PI uptake assay using tumor antigen expressing cells. According to this assay, tumor cell antigen expressing cells are cultured in Dulbecco's Modified Eagle Medium (D-MEM):Ham's F-12 (50:50) supplemented with 10% heat-inactivated FBS (Hyclone) and 2 mM L-glutamine. (Thus, the assay is performed in the absence of complement and immune effector cells). The tumor cells are seeded at a density of 3×10⁶ per dish in 100×20 mm dishes and allowed to attach overnight. The medium is then removed and replaced with fresh medium alone or medium containing 10 μg/mL of the appropriate monoclonal antibody. The cells are incubated for a 3 day time period. Following each treatment, monolayers are washed with PBS and detached by trypsinization. Cells are then centrifuged at 1200 rpm for 5 minutes at 4° C., the pellet resuspended in 3 mL ice cold Ca²⁺ binding buffer (10 mM Hepes, pH 7.4, 140 mM NaCl, 2.5 mM CaCl₂) and aliquoted into 35 mm strainer-capped 12×75 tubes (1 mL per tube, 3 tubes per treatment group) for removal of cell clumps. Tubes then receive PI (10 μg/mL). Samples may be analyzed using a FACSCAN™ flow cytometer and FACSCONVERT™. CellQuest software (Becton Dickinson). Those antibodies that induce statistically significant levels of cell death as determined by PI uptake may be selected as cell death-inducing antibodies.

Antibodies can also be screened in vivo for apoptotic activity using ¹⁸F-annexin as a PET imaging agent. In this procedure, Annexin V is radiolabeled with ¹⁸F and given to the test animal following dosage with the antibody under investigation. One of the earliest events to occur in the apoptotic process is the eversion of phosphatidylserine from the inner side of the cell membrane to the outer cell surface, where it is accessible to annexin. The animals are then subjected to PET imaging (see Yagle et al, J Nucl Med. 2005 April; 46(4):658-66). Animals can also be sacrificed and individual organs or tumors removed and analyzed for apoptotic markers following standard protocols.

While in some embodiments cancer may be characterized by overexpression of a gene expression product, the present application further provides methods for treating cancer which is not considered to be a tumor antigen-overexpressing cancer. To determine tumor antigen expression in the cancer, various diagnostic/prognostic assays are available. In some embodiments, gene expression product overexpression can be analyzed by IHC. Paraffin embedded tissue sections from a tumor biopsy may be subjected to the IHC assay and accorded a tumor antigen protein staining intensity criteria as follows:

Score 0: no staining is observed or membrane staining is observed in less than 10% of tumor cells.

Score 1+: a faint/barely perceptible membrane staining is detected in more than 10% of the tumor cells. The cells are only stained in part of their membrane.

Score 2+: a weak to moderate complete membrane staining is observed in more than 10% of the tumor cells.

Score 3+: a moderate to strong complete membrane staining is observed in more than 10% of the tumor cells.

In some embodiments those tumors with 0 or 1+ scores for tumor antigen overexpression assessment may be characterized as not overexpressing the tumor antigen, whereas those tumors with 2+ or 3+ scores may be characterized as overexpressing the tumor antigen.

In some embodiments, AMIGO-2 is not upregulated significantly in cancer cells as compared to normal cells, yet there is a differential dependence of cancer cells and normal cells on AMIGO-2 expression. In some embodiments, AMIGO-2 modulation affects tumor-stromal interactions. In some embodiments, inhibition of AMIGO-2 modulates tumor-stromal interactions.

Alternatively, or additionally, FISH assays such as the INFORM™ (sold by Ventana, Ariz.) or PATHVISION™ (Vysis, Ill.) may be carried out on formalin-fixed, paraffin-embedded tumor tissue to determine the extent (if any) of tumor antigen overexpression in the tumor.

Additionally, antibodies can be chemically modified by covalent conjugation to a polymer to increase their circulating half-life, for example. Each antibody molecule may be attached to one or more (i.e. 1, 2, 3, 4, 5 or more) polymer molecules. Polymer molecules are, in some embodiments, attached to antibodies by linker molecules. The polymer may, in general, be a synthetic or naturally occurring polymer, for example an optionally substituted straight or branched chain polyalkene, polyalkenylene or polyoxyalkylene polymer or a branched or unbranched polysaccharide, e.g. homo- or hetero-polysaccharide. In some embodiments the polymers are polyoxyethylene polyols and polyethylene glycol (PEG). PEG is soluble in water at room temperature and has the general formula: R(O—CH₂—CH₂)ₙO—R where R can be hydrogen, or a protective group such as an alkyl or alkanol group. In some embodiments, the protective group has between 1 and 8 carbons. In some embodiments the protective group is methyl. The symbol n is a positive integer, between 1 and 1,000, or 2 and 500. In some embodiments the PEG has an average molecular weight between 1000 and 40,000, between 2000 and 20,000, or between 3,000 and 12,000. In some embodiments, PEG has at least one hydroxy group. In some embodiments the hydroxy is a terminal hydroxy group. In some embodiments it is this hydroxy group which is activated to react with a free amino group on the inhibitor. However, it will be understood that the type and amount of the reactive groups may be varied to achieve a covalently conjugated PEG/antibody of the present invention. Polymers, and methods to attach them to peptides, are shown in U.S. Pat. Nos. 4,766,106; 4,179,337; 4,495,285; and 4,609,546 each of which is hereby incorporated by reference in its entirety.

Safety Studies

The antibodies of the invention can be examined for safety and toxicological characteristics. Guidelines for these types of studies can be found in the document issued by the USDA CBER division, “Points to Consider in the Manufacture and Testing of Monoclonal Antibody Products for Human Use” (Docket No. 94D-0259, Feb. 28, 1997) incorporated herein by reference. In general, the candidate antibodies should be screened in preclinical studies using a number of human tissue samples and/or isolated human cell types to assess non-target tissue binding and cross reactivity. Following a satisfactory outcome from these human tissue studies, a panel of tissue samples or isolated cells from a variety of animal species can be screened to identify a suitable species for use in general toxicological studies. If no cross reactive animal species is identified, other types of models may be deemed appropriate. These other models can include studies such as xenograft models, where human tumor cells are implanted into a rodent host, or the use of a surrogate monoclonal antibody which recognizes the corresponding tumor-cell antigen in the animal species chosen for the toxicological studies. It should be appreciated that the data from these types of alternate models will be first approximations and proceeding into higher species should be done with caution.

For a candidate naked antibody, studies looking at simple tolerability can be performed. In these studies the therapeutic index of the candidate molecule can be characterized by observing any dose-dependent pharmacodynamic effects. A broad range of doses should be used (for example from 0.1 mg/kg to 100 mg/kg). Differences between tumor cell antigen number, affinity of the candidate antibody for the cross reactive animal target and differences in cellular response following binding of the antibody should be considered in estimating therapeutic index. Pharmacodynamic and pharmacokinetic studies should also be carried out in an appropriate animal model to help guild initial dose considerations when the candidate antibody is tested in humans.

For candidate immunoconjugates, stability studies of the conjugate must be performed in vivo. Optimally, pharmacodynamic and pharmacokinetic studies should be carried out on the individual components of the immunoconjugate to determine the consequences of any breakdown products from the candidate immunoconjugate. Pharmacodynamic and pharmacokinetic studies should also be carried out as above in an appropriate animal model to help guild initial dose considerations. Additional consideration must be given to safety study design when the drug will be given in combination with pretreatment with naked antibody. Safety studies must be carried out with the naked antibody alone, and studies must be designed with the immunoconjugate keeping in mind that the ultimate doses of immunoconjugate will be lower in this type of treatment regimen.

For radio-immunoconjugates, animal tissue distribution studies should be carried out to determine biodistribution data. In addition, an accounting of metabolic degradation of the total dose of administered radioactivity should be performed with both early and late time points being taken. Radioimmunoconjugates can be tested for stability in vitro using serum or plasma, and methods should be developed to measure the percentages of free radionuclide, radioimmunoconjugate and labeled, non-antibody compounds.

Oligonucleotides

In some embodiments, the AMIGO-2 modulator is an oligonucleotide. In some embodiments, the AMIGO-2 modulator is an oligonucleotide comprising a sequence selected from the group consisting of SEQ ID NOS:7-24.

In some embodiments the oligonucleotide is an antisense or RNAi oligonucleotide. In some embodiments the oligonucleotide is complementary to a region, domain, portion, or segment of the AMIGO-2 gene or gene expression product. In some embodiments, the oligonucleotide comprises from about 5 to about 100 nucleotides, from about 10 to about 50 nucleotides, from about 12 to about 35, and from about 18 to about 25 nucleotides. In some embodiments, the oligonucleotide is at least 50%, at least 60%, at least 70%, at least 80%, at least 90%, at least 95%, at least 96%, at least 97%, at least 98%, at least 99%, or 100% homologous to a region, portion, domain, or segment of the AMIGO-2 gene or gene expression product. In some embodiments there is substantial sequence homology over at least 15, 20, 25, 30, 35, 40, 50, or 100 consecutive nucleotides of the AMIGO-2 gene or gene expression product. In some embodiments there is substantial sequence homology over the entire length of the AMIGO-2 gene or gene product. In some embodiments, there is full sequence identity over at least 15, 20, 25, 30, 35, 40, 50 or 100 consecutive nucleotides of the AMIGO-2 gene or gene expression product. In some embodiments, there is full sequence identity over the entire length of the AMIGO-2 gene or its gene product. In some embodiments, the oligonucleotide binds under moderate or stringent hybridization conditions to a nucleic acid molecule having a nucleotide sequence of SEQ ID NO:1.

In some embodiments, the AMIGO-2 modulator is a double stranded RNA (dsRNA) molecule and works via RNAi (RNA interference). In some embodiments, one strand of the dsRNA is at least 50%, at least 70%, at least 80%, at least 90%, at least 95%, at least 96%, at least 97%, at least 98%, at least 99%, or 100% homologous to a region, portion, domain, or segment of the AMIGO-2 gene. In some embodiments there is substantial sequence homology over at least 15, 20, 25, 30, 35, 40, 50, 100, 200, 300, 400, 500, or 1000 consecutive nucleotides of the AMIGO-2 gene. In some embodiments there is substantial sequence homology over the entire length of the AMIGO-2 gene.

In some embodiments, the AMIGO-2 modulator is a double stranded RNA (dsRNA) molecule (works via RNAi (RNA interference)), in which one or both strands of the dsRNA is partially complementary (e.g., at least 50%, at least 70%, at least 80%, at least 90%, at least 95%, at least 96%, at least 97%, at least 98%, at least 99%) complementary to a region, portion, domain, or segment of the AMIGO-2 gene. In some embodiments, one or both strands of the dsRNA is fully complementary to a region, portion, domain, or segment of the AMIGO-2 gene. Sequence “complementarity” refers to the chemical affinity between specific nitrogenous bases as a result of their hydrogen bonding properties (i.e., the property of two nucleic acid chains having base sequences such that an antiparallel duplex can form where the adenines and uracils (or thymine, in the case of DNA or modified RNA) are apposed to each other, and the guanines and cytosines are apposed to each other). Fully complementary sequences, thus, would be two sequences that have complete one-to-one correspondence (i.e., adenine to uracil and guanine to cytosine) of the base sequences when the nucleotide sequences form an antiparallel duplex.

In some embodiments oligonucleotides of the invention are used in a polymerase chain reaction (PCR). This sequence may be based on (or designed from) a genomic sequence or cDNA sequence and is used to amplify, confirm, or detect the presence of an identical, similar, or complementary DNA or RNA in a particular cell or tissue.

Small Molecules

In some embodiments, the AMIGO-2 modulator is a small molecule. As used herein, the term “small molecule” refers to an organic or inorganic non-polymer compound that has a molecular weight that is less than about 10 kilodaltons. Examples of small molecules include peptides, oligonucleotides, organic compounds, inorganic compounds, and the like. In some embodiments, the small molecule has a molecular weight that is less than about 9, about 8, about 7, about 6, about 5, about 4, about 3, about 2, or about 1 kilodalton.

Mimetics

In some embodiments, the AMIGO-2 modulator is a mimetic. As used herein, the term “mimetic” is used to refer to compounds which mimic the activity of a peptide. Mimetics are non-peptides but may comprise amino acids linked by non-peptide bonds. U.S. Pat. No. 5,637,677, issued on Jun. 10, 1997, and parent applications thereof, all of which are incorporated herein by reference, contain detailed guidance on the production of mimetics. Briefly, the three-dimensional structure of the peptides which specifically interact with the three dimensional structure of AMIGO-2 is duplicated by a molecule that is not a peptide. In some embodiments the AMIGO-2 mimetic is a mimetic of AMIGO-2 or a mimetic of a ligand of AMIGO-2.

Decoy Receptors

In some embodiments, the AMIGO-2 modulator is a decoy receptor comprising at least a portion of an AMIGO-2 receptor. In some embodiments the decoy receptor competes with natural AMIGO-2 receptors for AMIGO-2 ligands. In some embodiments, the decoy receptor is labeled to facilitate quantification, qualification, and/or visualization. In other embodiments, the decoy receptor further comprises a moiety to facilitate isolation and/or separation of the decoy receptor and or the decoy receptor-AMIGO-2 complex. In some embodiments, the decoy receptor, upon binding with an AMIGO-2 receptor ligand, causes an increased signal (compared to a native AMIGO-2 receptor) to be effected. In some embodiments, the decoy receptor is a non-signaling molecule which functions by capturing AMIGO-2 ligand and preventing it from interacting with the signaling AMIGO-2 receptor. In some embodiments the decoy receptor comprises at least a portion of an AMIGO-2 receptor fused to an antibody or antibody fragment.

Methods of Treating/Preventing Cancer

The present invention provides methods for treating and/or preventing cancer or symptoms of cancer in a subject comprising administering to the subject a therapeutically effective amount of one or more AMIGO-2 modulators of the present invention. In some embodiments the cancer is a cancer associated with overexpression of AMIGO-2. In some embodiments, the cancer is lung, bladder, kidney, colon, breast, uterine, ovarian, or pancreatic cancer. In some embodiments, the cancer is lung or colon cancer. In some embodiments the subject has been diagnosed as having a cancer or as being predisposed to cancer. In some embodiments, subject has been diagnosed as having a cancer or as being predisposed to a cancer other than gastric cancer.

Symptoms of cancer are well-known to those of skill in the art and include, without limitation, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreas metastases, difficulty swallowing, and the like.

A therapeutically effective amount of the modulating compound can be determined empirically, according to procedures well known to medicinal chemists, and will depend, inter alia, on the age of the patient, severity of the condition, and on the ultimate pharmaceutical formulation desired. Administration of the modulators of the present invention can be carried out, for example, by inhalation or suppository or to mucosal tissue such as by lavage to vaginal, rectal, urethral, buccal and sublingual tissue, orally, topically, intranasally, intraperitoneally, parenterally, intravenously, intralymphatically, intratumorly, intramuscularly, interstitially, intra-arterially, subcutaneously, intraoccularly, intrasynovial, transepithelial, and transdermally. In some embodiments, the inhibitors are administered by lavage, orally or inter-arterially. Other suitable methods of introduction can also include rechargeable or biodegradable devices and slow or sustained release polymeric devices. As discussed above, the therapeutic compositions of this invention can also be administered as part, of a combinatorial therapy with other known anti-cancer agents or other known anti-bone disease treatment regimen.

The present invention further provides methods of modulating an AMIGO-2-related biological activity in a patient. The methods comprise administering to the patient an amount of an AMIGO-2 modulator effective to modulate one or more AMIGO-2 biological activities. Suitable assays for measuring AMIGO-2 biological activities are set forth supra and infra.

The present invention also provides methods of inhibiting cancer cell growth in a patient in need thereof comprising administering a therapeutically effective amount of one or more AMIGO-2 modulators to the patient. Suitable assays for measuring AMIGO-2-related cell growth are known to those skilled in the art and are set forth supra and infra.

The present invention also provides methods of inhibiting cancer cell growth (e.g., in a patient in need of such a method) by administering to a patient having a cancer comprising one or more cells expressing AMIGO-2 a compound that modulates of one or more downstream markers of AMIGO-2. The one or more downstream markers of AMIGO-2 can be selected from the group consisting of c-MYC, c-Jun, FosL1, or Extracellular signal-Regulated Kinase (ERK). Modulation of ERK can be modulation of the phosphorylation of ERK or the phosphorylation by ERK of one or more of its substrates (see above). The method can optionally include the step of identifying a patient as having a cancer comprising one or more cells expressing AMIGO-2 (e.g., AMIGO-2 mRNA or AMIGO-2 protein).

The present invention further provides methods of inhibiting cancer in a patient in need thereof. The methods comprise determining if the patient is a candidate for AMIGO-2 therapy as described herein and administering a therapeutically effective amount of one or more AMIGO-2 modulators to the patient if the patient is a candidate for AMIGO-2 therapy. If the patient is not a candidate for AMIGO-2 therapy, the patient is treated with conventional cancer treatment.

The present invention further provides methods of inhibiting cancer in a patient diagnosed or suspected of having a cancer. The methods comprise administering a therapeutically effective amount of one or more AMIGO-2 modulators to the patient.

The present invention also provides methods for inhibiting the interaction of two or more cells in a patient comprising administering a therapeutically effective amount of an AMIGO-2 modulator to said patient. Suitable assays for measuring AMIGO-2-related cell interaction are known to those skilled in the art and are set forth supra and infra.

The present invention also provides methods of modulating one or more symptoms of cancer in a patient comprising administering to said patient a therapeutically effective amount of one or more AMIGO-2 modulators.

The present invention further provides methods for inhibiting cell growth in a patient in need thereof comprising administering to the patient a therapeutically effective amount of an AMIGO-2 modulator. Suitable assays for measuring cell growth are known to those skilled in the art and are set forth supra and infra.

The present invention also provides methods for inhibiting migration of cancer cells in a patient in need thereof comprising administering to the patient a therapeutically effective amount of an AMIGO-2 modulator. Suitable assays for measuring AMIGO-2-related cell migration are known to those skilled in the art and are set forth supra and infra.

The present invention further provides methods for inhibiting adhesion of cancer cells in a patient in need thereof comprising administering to the patient a therapeutically effective amount of an AMIGO-2 modulator. Suitable assays for measuring AMIGO-2-related cell adhesion are known to those skilled in the art and are set forth supra and infra.

The present invention also provides methods for inhibiting angiogenesis in a patient in need thereof comprising administering to the patient a therapeutically effective amount of an AMIGO-2 modulator. Suitable assays for measuring angiogenesis are known to those skilled in the art and are set forth supra and infra.

The present invention also provides methods to prophylactically treat a patient who is predisposed to develop cancer, a cancer metastasis or who has had a metastasis and is therefore susceptible to a relapse or recurrence. The methods are particularly useful in high-risk individuals who, for example, have a family history of cancer or of metastasizing tumors, or show a genetic predisposition for a cancer metastasis. In some embodiments the tumors are AMIGO-2-related tumors. Additionally, the methods are useful to prevent patients from having recurrences of AMIGO-2-related tumors who have had AMIGO-2-related tumors removed by surgical resection or treated with a conventional cancer treatment.

The present invention also provides methods of inhibiting cancer progression and/or causing cancer regression comprising administering to the patient a therapeutically effective amount of an AMIGO-2 modulator.

In some embodiments, the patient in need of anti-cancer treatment is treated with the AMIGO-2 modulators of the present invention in conjunction with chemotherapy and/or radiation therapy. For example, following administration of the AMIGO-2 modulators, the patient may also be treated with a therapeutically effective amount of anti-cancer radiation. In some embodiments chemotherapeutic treatment is provided in combination with AMIGO-2 modulators. In some embodiments AMIGO-2 modulators are administered in combination with chemotherapy and radiation therapy.

Methods of treatment comprise administering single or multiple doses of one or more AMIGO-2 modulators to the patient. In some embodiments the AMIGO-2 modulators are administered as injectable pharmaceutical compositions that are sterile, pyrogen free and comprise the AMIGO-2 modulators in combination with a pharmaceutically acceptable carrier or diluent.

In some embodiments, the therapeutic regimens of the present invention are used with conventional treatment regimens for cancer including, without limitation, surgery, radiation therapy, hormone ablation and/or chemotherapy. Administration of the AMIGO-2 modulators of the present invention may take place prior to, simultaneously with, or after conventional cancer treatment. In some embodiments, two or more different AMIGO-2 modulators are administered to the patient.

In some embodiments the amount of AMIGO-2 modulator administered to the patient is effective to inhibit one or more of chromosomal instability, kinase activity, tumorigenicity, metastasis, AMIGO-2 signaling, cell adhesion, cancer cell survival, ERK phosphorylation, cancer cell growth, tumor formation, cyclin production, cell proliferation, progression through the cell cycle, anchorage-independent growth, localization of AMIGO-2 protein to the cell membrane, interactions between AMIGO-2 and one or both of AMIGO-1 or AMIGO-3, levels of cytoplasmic phosphorylated AMIGO-2 protein, and angiogenesis, among others. In some embodiments the amount of AMIGO-2 modulator administered to the patient is effective to increase cancer cell death through apoptosis.

Methods of Treating Diseases and Disorders Relating to the Nervous System

The present invention also provides methods for treating diseases and disorders of the nervous system in a patient in need thereof comprising administering to the patient a therapeutically effective amount of an AMIGO-2 modulator. In some embodiments, the present invention provides methods for treating one or more of Alzheimers disease, Parkinsons Disease, epilepsy, multiple sclerosis, Huntington Disease, spinal cord injury, stroke, facial nerve damage, diabetes-related nerve damage, and retinal degeneration.

Methods of Perturbing Downstream Gene Expression

In some embodiments, the present invention provides methods of perturbing one or more genes. In some embodiments the method comprises contacting a cell which overexpresses AMIGO-2 with an AMIGO-2 modulator. In some embodiments the expression of one or more genes are perturbed in vivo following administration of a therapeutically effective amount of an AMIGO-2 modulator in the patient. In some embodiments the AMIGO-2 modulator inhibits expression of one or more genes selected from the group consisting of BNIP3L, FAM46C, LOC339988, SATB1, C11orf21, FAM3B, UCP2, FNDC3A, DRE1, PP1C, ASS, KIAA1718, ALDH6A1, LR8, ADAMTSL2, LIPC, FZD10, COL4A2, TPP1, SERPINF1, ADCY9, AZGP1, USH1C, RAB40B, SMOC2, RRAGD, NUDT21, C14orf1, TPP1, RPS6 KB1, KIAA0657, QPRT, RFK, KCNH2, PAQR8, SEPT6, LOC285758, BFNA1, LGALS8, ATP10D, ERBB3, CHST13, FLJ25476, MCF2L, FLJ10159, RAB13, ZNF261, USH1C, OSTM1, BMPR2, IPO9, ZNF226, HRASLS3, ERBB3, PROS1, ALDH6A1, PPT2, TFF3, C21orf86, LRRC8B, BAZ2B, HIP1, RNASE4, FAM46C, STARD10, KLF13, BACE1, FCGRT, QPCT, KCTD14, FRAS1, FAM63B, SPON2, IQWD1, BMPR2, TXNIP, ZBTB4, DDAH2, ZNF420, LGALS8, NEU1, FUCA1, GRN, C20orf194, SEPT6, ASPSCR1, KLHDC5, GRN, STARD10, MGC12981, C14orf1, EPB41, ALDH2, ARHGEF10L, FNDC3A, CYP2R1, PAQR8, RNF38, KIAA1327, ALS2CR3, EPS8L3, BACE1, SEPT6, IL27RA, DTX3L, CBPIN, ZNF627, C1orf85, AZGP1, GRN, SUOX, PSMB8, ARHGAP1, DACH1, COL4A2, PLEKHB1, SEC14L1, C2orf7, TPD52L1, PGM2, C14orf4, ZNF286, CAMK2D, PSME1, ZNF268, TRAF5, SEPT6, MGC45474, WIPI-2, CAMK2D, ZBED1, SEC24A, GGTL3, TRIM4, PPM1H, JMJD1A, DOK4, RPS6KB1, PSAP, EXOC7, C6orf80, RERE, ZNF641, MXRA7, RAC1, NDST1, JAG2, ZNF329, SEPT6, KLHDC5, STXBP1 and UBE2L3, and combinations and subcombinations thereof.

Combination Therapy

In some embodiments the invention provides compositions comprising two or more AMIGO-2 modulators to provide still improved efficacy against cancer. In some embodiments the AMIGO-2 modulators are monoclonal antibodies. Compositions comprising two or more AMIGO-2 antibodies may be administered to persons or mammals suffering from, or predisposed to suffer from, cancer. One or more antibodies may also be administered with another therapeutic agent, such as a cytotoxic agent, or cancer chemotherapeutic. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some embodiments administration of combinations, or “cocktails”, of different antibodies is contemplated. Such antibody cocktails may have certain advantages inasmuch as they contain antibodies which exploit different effector mechanisms or combine directly cytotoxic antibodies with antibodies that rely on immune effector functionality. Such antibodies in combination may exhibit synergistic therapeutic effects.

A cytotoxic agent refers to a substance that inhibits or prevents the function of cells and/or causes destruction of cells. The term is intended to include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody according to the invention.

In some embodiments, conventional cancer medicaments are administered with the compositions of the present invention. Conventional cancer medicaments include:

a) cancer chemotherapeutic agents;

b) additional agents;

c) prodrugs.

Cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabinc (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

In some embodiments, one or more AMIGO-2 modulators could be administered in conjunction with one or more conventional immunotherapy agents for cancer. Immunotherapy agents can include those that aim to stimulate the immune system (e.g., stimulate the production or activity of natural killer cells, macrophages, and neutrophils) such as Interferon alfa, granulocyte-monocyte colony stimulating factor (GM-CSF), Interleukin-12, or Interleukin-2. The immunotherapy agents can also include one or more vaccine agents useful for treating a cancer. For example, one or more AMIGO-2 modulators can be administered with (e.g., at the same time, around the same time, or as one component of the overall treatment strategy for a patient) peptide, polypeptide, or viral vector vaccines for a given cancer or cancer antigen (e.g., a MAGE antigen). The one or more AMIGO-2 modulators can also be administered with one or more monoclonal antibody therapies (e.g., agents) targeted to particular cancers or cancer antigens. For example, Rituximab (IDEC-C2B8)—a chimeric antibody which targets the CD20 antigen present in certain B cell malignancies binds to Fc receptors on immune effector cells (e.g., monocytes, macrophages, or natural killer cells) and promotes the destruction of tumor cells by these immune effector cells (also called-antibody-dependent cell-mediated toxicity, see above). Antibodies that bind to specific tumor cells or tumor cell antigens can also be used to trigger the complement-dependent cytotoxicity response as described herein.

In some embodiments, one or more AMIGO-2 modulators can be administered in conjunction with one or more cell-cycle-targeting agents for cancer. Cell-cycle-targeting agents can include those that target specific protein mediators of the cell cycle (e.g., cyclin-dependent kinases) such as roscovitine or flavopiridol. Cell cycle-targeting agents (e.g., compounds) can also include agents that cause dividing cells (e.g., cancer cells) to arrest at particular phases of the cell cycle such as, but in no way limited to, taxol, staurosporin, UCN-01, roscovitine, or vinblastine.

In some embodiments, the one or more AMIGO-2 modulators and the one or more additional agents are administered at the same time. In other embodiments, the one or more AMIGO-2 modulators are administered first in time and the one or more additional agents are administered second in time. In some embodiments, the one or more additional agents are administered first in time and the AMIGO-2 modulator(s) is administered second in time. The one or more AMIGO-2 modulators can replace or augment a previously or currently administered therapy. For example, upon treating with one or more AMIGO-2 modulators, administration of the one or more additional agents can cease or diminish, e.g., be administered at lower levels. In other embodiments, administration of the previous therapy is maintained. In some embodiments, a previous therapy will be maintained until the level of the one or more AMIGO-2 modulators reach a level sufficient to provide a therapeutic effect (e.g., when determining the optimal dosage for a given patient). The two therapies can be administered in combination.

In some embodiments, the one or more AMIGO-2 modulators can be administered to a subject (e.g., a human patient) to offset the level or dosage of one or more agents being administered concurrently. For example, when a dosage of first therapy (e.g., one or more additional agents such as taxol) is toxic or poorly tolerated by a patient, the dosage can be lowered when one or more AMIGO-2 modulators are administered. Said administration of the one or more AMIGO-2 modulators providing equivalent or greater therapeutic effect to that of the first therapy without the toxic or poorly tolerated side-effects.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

Clinical Aspects

In some embodiments, the methods and compositions of the present invention are particularly useful in lung, bladder, kidney, colon, breast, uterine, ovarian, or pancreatic cancer and cancer metastases. In some embodiments, the cancer is lung or colon cancer.

Pharmaceutical Compositions

The present invention also provides pharmaceutical compositions comprising one or more of AMIGO-2 modulators and a pharmaceutically acceptable carrier. In some embodiments the pharmaceutical compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

Methods of Detecting AMIGO-2

The present invention also provides methods for detecting AMIGO-2. In some embodiments AMIGO-2 is present in a patient or in a patient sample. In some embodiments the method comprises administering a composition comprising one or more AMIGO-2 modulators to the patient and detecting the localization of the imaging agent in the patient. In some embodiments the patient sample comprises cancer cells. In some embodiments the AMIGO-2 modulator is linked to an imaging agent or is detectably labeled. In some embodiments, the AMIGO-2 modulator is an AMIGO-2 antibody conjugated to an imaging agent and is administered to a patient to detect one or more tumors or to determine susceptibility of the patient to AMIGO-2 therapy. The labeled antibodies will bind to the high density of receptors on cells and thereby accumulate on the tumor cells. Using standard imaging techniques, the site of the tumors can be detected.

The present invention also provides methods of imaging/detecting cells or tumors expressing or overexpressing AMIGO-2 comprising contacting a composition comprising an AMIGO-2 modulator to a sample and detecting the presence of the AMIGO-2 modulator in the sample. In some embodiments the sample is a patient sample. In some embodiments the patient sample comprises cancer cells. In some embodiments the AMIGO-2 modulator is linked to an imaging agent or is detectably labeled.

The present invention also provides methods for quantifying the amount of AMIGO-2 present in a patient, cell or sample. The methods comprise administering one or more of antibodies, probes, or small molecules to a patient or sample and detecting the amount of AMIGO-2 present in the sample. In some embodiments the antibodies, probes, or small molecules are linked to an imaging agent or are detectably labeled. Such information indicates, for example, whether or not a tumor is related to AMIGO-2, and, therefore, whether specific treatments should be used or avoided. In some embodiments, using standard techniques well known to the art-skilled, samples believed to include tumor cells are obtained and contacted with labeled antibodies, probes, oligonucleotides, and small molecules. After removing any unbound, labeled antibodies, probes, oligonucleotides or small molecules, the quantity of labeled antibodies, peptides, oligonucleotides or mimetics bound to the cell, or the quantity of antibodies, peptides, oligonucleotides or mimetics removed as unbound is determined. The information directly relates to the amount of AMIGO-2 present.

Imaging can be performed using procedures well known to those of ordinary skill in the art. Imaging can be performed, for example, by radioscintigraphy, nuclear magnetic resonance imaging (MRI) or computed tomography (CT scan). The most commonly employed radiolabels for imaging agents include radioactive iodine and indium. Imaging by CT scan may employ a heavy metal such as an iron chelate. MRI scanning may employ chelates of gadolinium or manganese. Additionally, positron emission tomography (PET) may be possible using positron emitters of oxygen, nitrogen, iron, carbon, or gallium.

In some embodiments the AMIGO-2 modulator is an AMIGO-2 antibody. In some embodiments the modulator is linked to an imaging agent or is detectably labeled. In some embodiments the imaging agent is ¹⁸F, ⁴³K, ⁵²Fe, ⁵⁷Co, ⁶⁷Cu, ⁶⁷Ga, ⁷⁷Br, ⁸⁷MSr, ⁸⁶Y, ⁹⁰Y, ⁹⁹MTc, ¹¹¹In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi.

Methods of detection are well known to those of skill in the art. For example, methods of detecting polynucleotides include, but are not limited to PCR, Northern blotting, Southern blotting, RNA protection, and DNA hybridization (including in situ hybridization). Methods of detecting polypeptides include, but are not limited to, Western blotting, ELISA, enzyme activity assays, slot blotting, peptide mass fingerprinting, electrophoresis, immunochemistry and immunohistochemistry. Other examples of detection methods include, but are not limited to, radioimmunoassay (RIA), chemiluminescence immunoassay, fluoroimmunoassay, time-resolved fluoroimmunoassay (TR-FIA), two color fluorescent microscopy, or immunochromatographic assay (ICA), all well known by those of skill in the art. In some embodiments of the present invention, polynucleotide expression is detected using PCR methodologies and polypeptide production is detected using ELISA technology.

Methods for Delivering a Cytotoxic Agent or a Diagnostic Agent to a Cell

The present invention also provides methods for delivering a cytotoxic agent or a diagnostic agent to one or more cells that express AMIGO-2. In some embodiments the methods comprise contacting an AMIGO-2 modulator of the present invention conjugated to a cytotoxic agent or diagnostic agent with the cell.

Methods for Determining the Prognosis of a Cancer Patient

The present invention also provides methods for determining the prognosis of a patient with an AMIGO-2-associated cancer. In some embodiments the methods comprise determining the ratio of levels of AMIGO-2 localized to the cell membrane compared to levels of AMIGO-2 localized to other areas of the cancer cells. In some embodiments, patients with a higher level of AMIGO-2 localized to the cell membrane than of AMIGO-2 localized to other areas of the cancer cells not including the cell membrane indicates that the patient has an AMIGO-2-related cancer and is susceptible to AMIGO-2 therapy. In some embodiments a ratio of AMIGO-2 localized to the cell membrane compared to AMIGO-2 localized to other areas of the cancer cells not including the cell membrane of at least 2:1 indicates that the patient has an AMIGO-2-related cancer and is susceptible to AMIGO-2 therapy. In some embodiments a ratio of AMIGO-2 localized to the cell membrane to AMIGO-2 localized to other areas of the cancer cells not including the cell membrane of at least 3:1 indicates that the patient has an AMIGO-2-related cancer and is susceptible to AMIGO-2 therapy.

Methods for Determining Susceptibility to AMIGO-2 Therapy

The present invention also provides methods for determining the susceptibility of a patient to AMIGO-2 therapy. The methods comprise detecting the presence or absence of evidence of differential expression of AMIGO-2 in a patient or patient sample. In some embodiments the presence of evidence of differential expression of AMIGO-2 in the patient or sample is indicative of a patient who is susceptible to AMIGO-2 therapy. In some embodiments, the absence of evidence of differential expression of AMIGO-2 in the patient or patient sample is indicative of a patient who is not a candidate for AMIGO-2 therapy. In some embodiments, AMIGO-2 is not up-regulated significantly in cancer cells as compared to normal cells, yet there is a differential dependence of cancer cells and normal cells on AMIGO-2 expression. In some embodiments, AMIGO-2 modulation affects tumor-stromal interactions. In some embodiments, AMIGO-2 modulation inhibits interactions between tumor and stromal tissues.

In some embodiments therapeutic methods comprise first identifying patients susceptible to AMIGO-2 therapy comprising administering to the patient in need thereof a composition comprising an AMIGO-2 modulator linked to an imaging agent and detecting the presence or absence of evidence of the gene or gene product in the patient. In some embodiments, the therapeutic methods further comprise administering one or more AMIGO-2 modulators to the patient if the patient is a candidate for AMIGO-2 therapy and treating the patient with conventional cancer treatment if the patient is not a candidate for AMIGO-2 therapy.

In some therapeutic methods, one or more AMIGO-2 modulators are administered to the patients alone or in combination with other anti-cancer medicaments when the patient is identified as having a cancer or being susceptible to a cancer.

Methods for Assessing the Progression of Cancer

The invention also provides methods for assessing the progression of cancer in a patient comprising comparing the level of an expression product of AMIGO-2 in a biological sample at a first time point to a level of the same expression product at a second time point. A change in the level of the expression product at the second time point relative to the first time point is indicative of the progression of the cancer.

Methods for Screening

The present invention also provides methods of screening for anti-cancer agents. The methods comprise contacting a cell expressing AMIGO-2 with a candidate compound and determining whether an AMIGO-2-related biological activity is modulated. In some embodiments, inhibition of one or more of chromosomal instability, kinase activity, tumorigenicity, cancer cell growth, cancer cell survival, tumor formation, cancer cell proliferation, metastasis, cell migration, substrate phosphorylation, cyclin production, angiogenesis, cell proliferation, cell cycle regulation, signaling, cell-cell adhesion, cell-cell membrane interaction, cell-extracellular matrix interaction, anchorage-independent growth, localization of AMIGO-2 protein to the cell membrane, interactions between AMIGO-2 and one or both of AMIGO-1 or AMIGO-3, and AMIGO-2 expression is indicative of an anti-cancer agent. In some embodiments, anti-cancer agents identified by the methods of the present invention are administered to patients in need thereof in therapeutic and/or diagnostic methods.

In some embodiments, the invention provides methods of screening for anti-cancer agents, particularly anti-metastatic cancer agents, by, for example, screening putative modulators for an ability to modulate the activity or level of a downstream marker. In some embodiments candidate agents that decrease levels of cyclin D1, cyclin B1, c-Myc, c-Jun, Extracellular signal-Regulated Kinase (ERK); Vascular Endothelial Growth Factor (VEGF), urokinase, and Poly(ADP-Ribose)Polymerase 1 (PARP1) are identified as anti-cancer agents.

In some embodiments, the invention provides methods for identifying an AMIGO-2 modulator. In some embodiments the method comprises comparing phosphorylation of AMIGO-2 in a sample comprising one or more cells expressing AMIGO-2 in the presence and absence of a candidate compound. In some embodiments modulation of phosphorylation of AMIGO-2 in the sample in the presence of the candidate compound as compared to phosphorylation of AMIGO-2 in the sample in the absence of the candidate compound indicates that the candidate compound is an AMIGO-2 modulator.

In some embodiments AMIGO-2 is isolated from the sample using an immunoprecipitating antibody. In some embodiments the immunoprecipitating antibody is an anti-AMIGO-2 antibody of the present invention. In some embodiments the AMIGO-2 phosphorylation is serine/threonine phosphorylation. In some embodiments AMIGO-2 phosphorylation is detected and/or quantified using a phosphoserine/threonine antibody. In some embodiments the immunoprecipitating antibody is a phosphoserine/threonine antibody.

Methods for Detecting Modulation of AMIGO-2

In some embodiments, the invention provides methods of detecting modulation of AMIGO-2 activity in cells. In some embodiments the methods comprise contacting a sample comprising cells which express AMIGO-2 with an AMIGO-2 inhibitor for a time sufficient to modulate AMIGO-2 activity, immunoprecipitating AMIGO-2 with an AMIGO-2 antibody of the present invention; and comparing AMIGO-2 serine/threonine phosphorylation in the sample to a control using a phospho-serine/threonine antibody. In some embodiments alteration of serine/threonine phosphorylation of AMIGO-2 in cells of the sample compared to a control is an indication of the modulation of AMIGO-2 activity. In some embodiments the AMIGO-2 phosphorylation is serine/threonine phosphorylation. In some embodiments AMIGO-2 phosphorylation is detected and/or quantified using a phosphoserine/threonine antibody. In some embodiments the immunoprecipitating antibody is a phosphoserine/threonine antibody.

In some embodiments, the invention provides methods of detecting modulation of AMIGO-2 activity in a sample comprising cells which overexpress AMIGO-2. In some embodiments the methods comprise overexpressing AMIGO-2 in the cells for a time sufficient to modulate AMIGO-2 activity, immunoprecipitating AMIGO-2 with an AMIGO-2 antibody of the present invention, and comparing AMIGO-2 serine/threonine phosphorylation in the sample to a control using a phosphoserine/threonine antibody. In some embodiments alteration of serine/threonine phosphorylation of AMIGO-2 in the sample compared to a control is an indication of the modulation of AMIGO-2 activity.

In some embodiments the methods comprise contacting the sample with an AMIGO-2 inhibitor for a time sufficient to modulate AMIGO-2 activity, immunoprecipitating AMIGO-2 with an anti-phospho-serine/threonine antibody; and comparing the level of phosphorylated AMIGO-2 in the sample to a control using an antibody of the present invention. In some embodiments alteration of the level of phosphorylated AMIGO-2 in the sample compared to the control is an indication of the modulation of AMIGO-2 activity.

In some embodiments the method comprises overexpressing AMIGO-2 in the sample for a time sufficient to modulate AMIGO-2 activity, immunoprecipitating AMIGO-2 with an anti-phospho-serine/threonine antibody and comparing the level of phosphorylated AMIGO-2 in the sample to a control using an AMIGO-2 antibody of the present invention. In some embodiments alteration of the level of phosphorylated AMIGO-2 in the sample compared to a control is an indication of the modulation of AMIGO-2 activity.

Methods for Purifying AMIGO-2

In some embodiments, the invention provides methods of purifying AMIGO-2 protein from a sample comprising AMIGO-2. The methods comprise providing an affinity matrix comprising an AMIGO-2 antibody of the present invention bound to a solid support, contacting the sample with the affinity matrix to form an affinity matrix-AMIGO-2 protein complex, separating the affinity matrix-AMIGO-2 protein complex from the remainder of the sample; and releasing AMIGO-2 protein from the affinity matrix.

Kits

In some embodiments, the present invention provides kits for imaging and/or detecting a gene or gene product correlated with differential expression of AMIGO-2. Kits of the invention comprise detectable antibodies, small molecules, oligonucleotides, decoys, mimetics or probes as well as instructions for performing the methods of the invention. Optionally, kits may also contain one or more of the following: controls (positive and/or negative), containers for controls, photographs or depictions of representative examples of positive and/or negative results.

Each of the patents, patent applications, accession numbers and publications described herein is hereby incorporated by reference in its entirety.

Various modifications of the invention, in addition to those described herein, will be apparent to those of skill in the art in view of the foregoing description. Such modifications are also intended to fall within the scope of the appended embodiments. The present invention is further demonstrated in the following examples that are for purposes of illustration and are not intended to limit the scope of the present invention.

### EXAMPLES

### Example 1

### AMIGO-2 Expression is Upregulated in Some Cancer Tissues

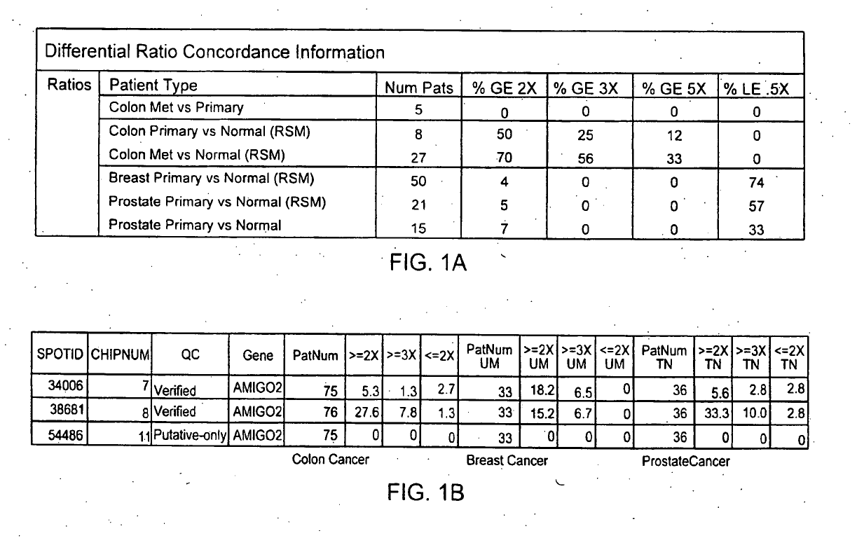

mRNA was isolated from laser capture microdissected (LCM) colon cancer, breast cancer and prostate cancer tissues, and the mRNA was compared to either a pool of respective normal tissue (FIG. 1A; RSM=reference standard mix) or normal cells adjacent to the cancer cells within each tissue sample (FIG. 1B). Samples were tested by oligonucleotide array analysis on either Affymetrix® GeneChips® (Affymetrix, Inc., Santa Clara, Calif.) (FIG. 1A) or arrays that were generated in-house using cDNA libraries made from cancerous tissue (FIG. 1B). For each table in FIGS. 1A and 1B, the number of patients is indicated, followed by the relative expression between the cancer and the normal samples. (“% GE2X” and “>=2X” denote up-regulation by 2-fold; “% LE 0.5X” and “<=2x” denotes down-regulation by 2-fold). Both sets of chips demonstrated that AMIGO-2 is up-regulated in colon cancer. Experiments performed with in-house chips indicated that AMIGO-2 is also upregulated in breast and prostate cancers. Upregulation in these tissues was not observed in the Affymetrix experiments.

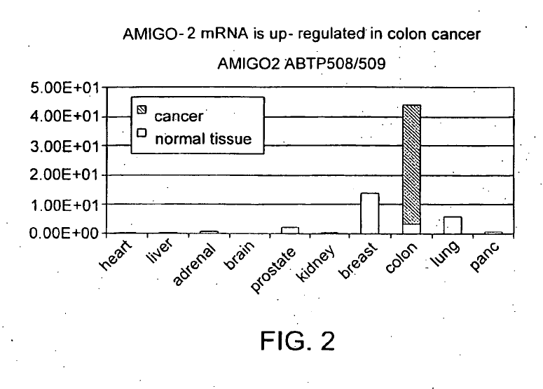

Reverse-transcription-coupled polymerase chain reaction (RT-PCR) was also used to examine relative AMIGO-2 mRNA levels in normal and cancer tissue samples (FIG. 2). A panel of normal tissues and pools of colon, breast and prostate LCM (laser capture dissection) dissected samples (8 patients per pool) were compared by semi-quantitative RT-PCR (GeneAmp®, Applied Biosystems, Foster City, Calif.). Two primer sets were tested with similar results (data from primer set named “ABTP 508/509” is shown in FIG. 2). Among the normal tissues tested, breast and lung showed the highest relative expression. Colon cancer showed a greater than 5-fold up-regulation over normal colon and several-fold up-regulation over normal breast.

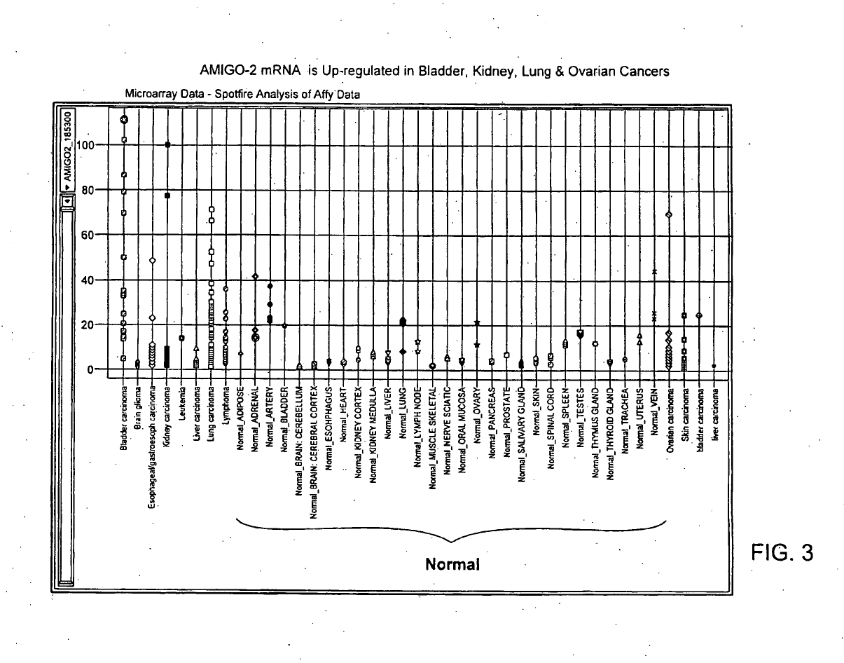

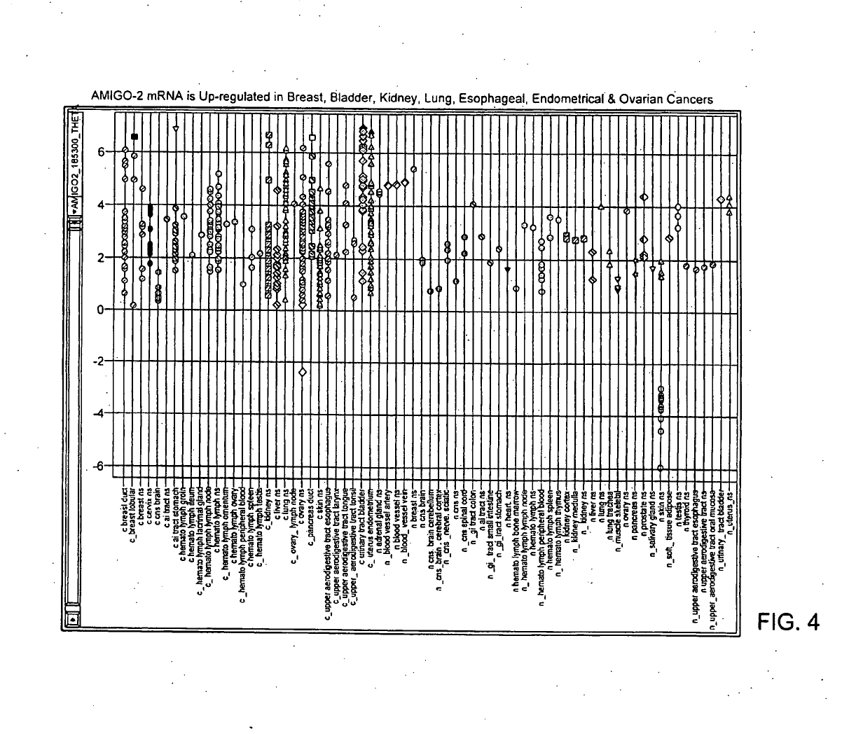

A graphical representation of an oligonucleotide array analysis of AMIGO-2 mRNA expression in cancerous and normal tissues using a Human Genome U133 Plus 2.0 Array (Affymetrix, Inc.) is shown in FIGS. 3 and 4. Normal and cancerous tissue types are presented along the horizontal axis. In FIG. 4, cancerous tissues are labeled with a ‘c_’ (e.g., “c_breast_duct,” which represents a breast cancer tissue sample), and normal tissues are labeled with an ‘n_’. The tissue types are further labeled with respect to the type and subtype of the tissue, if known. For example, “c_breast_duct” is a cancerous tissue from a breast cancer that was localized in a breast duct. If the subtype was not clear during surgical removal or was unknown, the label includes, ‘ns’ for ‘non-specified.’ Each spot on the vertical axes of FIGS. 3 and 4 represents a tissue sample from a single patient, and the height of each spot on the vertical axes (linear) represents the relative expression level of the probeset. FIG. 3 represents a linear analysis, while the data in FIG. 4 is presented on a log₂ scale. Filled circles represent samples with expression levels in the linear detection range. Open circles represent an upper limit on gene expression in samples where the gene was below the probeset's detection limit. Open squares represent a lower limit on gene expression in samples where the probeset was saturated. Before performing an analysis, each probeset was calibrated by analyzing the behavior of its constituent probes across a large, diverse set of samples. This calibration measured the relative sensitivity of each probe, and the range of intensities within which the probeset response was linear between probes. Intensities below this range are called “undetected” while those above it are called “saturated.” Because of variation in the hybridization and labeling efficiency between samples, each array was normalized after applying the calibrations. This caused the upper and lower limits of the range, in terms of gene expression, to vary somewhat from sample to sample.

### Example 2

### Immunohistochemistry Reveals that AMIGO-2 is Expressed in Colon and Lung Cancers

Tissue sections were deparaffinized and antigen retrieval was performed on a Ventana Discovery instrument (Ventana Medical Systems, Inc., Tucson, Ariz.). Standard cell conditioning was performed, and then cells were incubated for 60 minutes with primary antibodies. A rabbit anti-human AMIGO-2 antibody (Chiron, Emeryville, Calif.) and rabbit IgG Prebleed control (Chiron, Emeryville, Calif.) were used at 10 μg/ml. Ventana Universal Secondary Reagent (Ventana Medical Systems, Inc.) followed by Ventana DAB Map Kit (Ventana Medical Systems, Inc.) was used for detection. Ventana Hematoxylin and Bluing Reagents (Ventana Medical Systems, Inc.) were used for counterstain, and sections were dehydrated in graded alcohols, cleared in xylene and coverslipped using a synthetic mounting media.

Staining indicated that AMIGO-2 is expressed in tumor cells and the tumor stroma. AMIGO-2 expression in stromal tissue surrounding the tumors was observed to be equivalent to or greater than expression in the tumors themselves, indicating that AMIGO-2 may be important for vascularization required to support tumor growth.

### Example 3

### AMIGO-2 Protein Levels Vary in Different Cell Lines

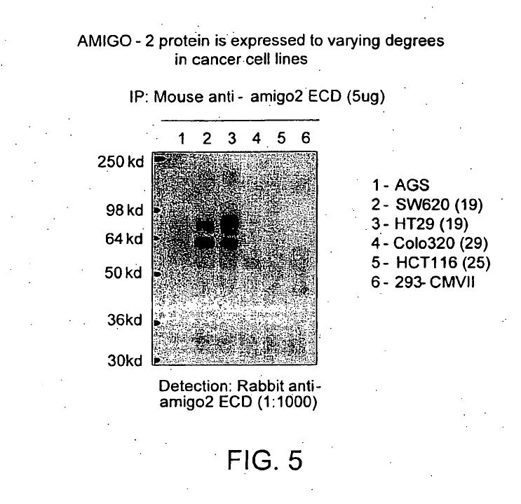

Protein lysates were made from cell pellets of different cell lines, and the lysates were subjected to immunoprecipitation with a commercially available AMIGO-2 antibody (MAB2080 from R&D Systems, Inc., Minneapolis, Minn.), which specifically recognizes AMIGO-2, but not AMIGO-1 or AMIGO-3 proteins. Cell lines included a human gastric cancer line (AGS), two colon cancer lines (SW620 and HT29), two colorectal lines (Colo320 and HCT116) and an embryonic cell line (293-CMVII) (FIG. 5). Proteins captured by immunoprecipitation were separated by acrylamide gel electrophoresis and then subjected to Western analysis using an in-house generated anti-AMIGO-2 antibody (FIG. 5). Two bands (˜0.63 kD & ˜90 kD) were observed in the colon and gastric cell lines. The higher molecular weight product may be a glycosylated, phosphorylated or multimeric form of AMIGO-2. Relative semi-quantitative RT-PCR Ct levels were determined and are indicated in parentheses adjacent to the names of four of the cell lines in FIG. 5. The Ct value indicates the threshold of detection following a certain number of cycles of PCR. A higher Ct value indicates that a higher number of cycles is required to detect the cDNA, and is therefore indicative of lower mRNA levels. All the cell lines tested were positive for AMIGO-2 mRNA, but the ability to detect protein decreased with decreasing amounts of mRNA. FIG. 5 indicates that AMIGO-2 is expressed at significant levels in colon cancer cell lines.

### Example 4

### Functional Assays

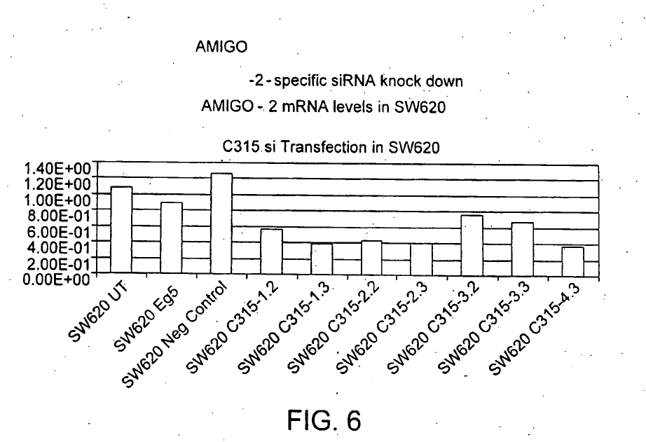

A panel of siRNAs was tested for the ability to knock down AMIGO-2 mRNA in SW620 cells (a colon cancer cell line expressing AMIGO-2) (FIG. 6). The sequences of the siRNAs tested in FIG. 6 are presented in Table 3. The AMIGO-2 siRNAs shown in FIG. 6 all reduced AMIGO-2 mRNA levels to some degree, but siRNA agents C315-1.3 and C315-4.3 appeared to be the most efficient in reducing mRNA levels.

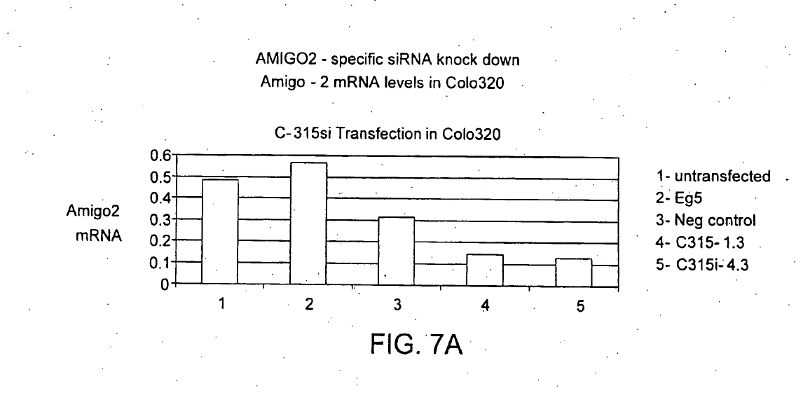

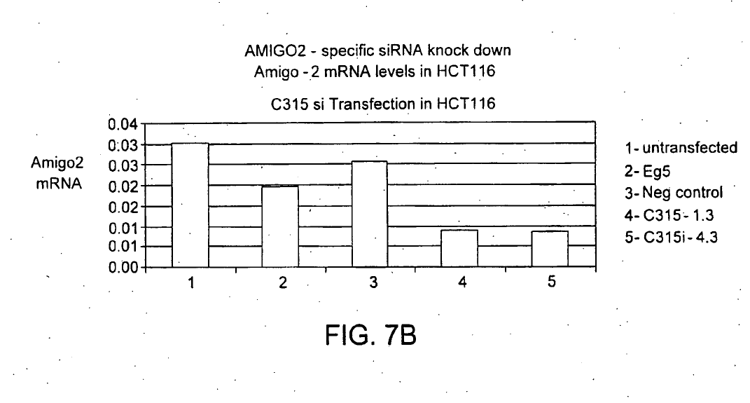

Although AMIGO-2 protein levels were not detectable by Western blot analysis in Colo320 and HCT116 cells (see FIG. 5), AMIGO-2-specific siRNAs C315-1.3 and C315-4.3 reduced AMIGO-2 mRNA levels in these cell lines (FIGS. 7A and 7B), consistent with the positive mRNA expression in Colo320 and HCT116 cells.

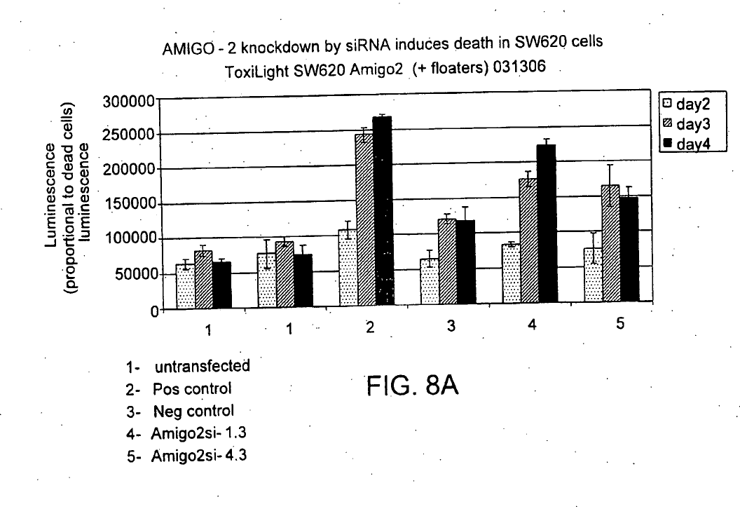

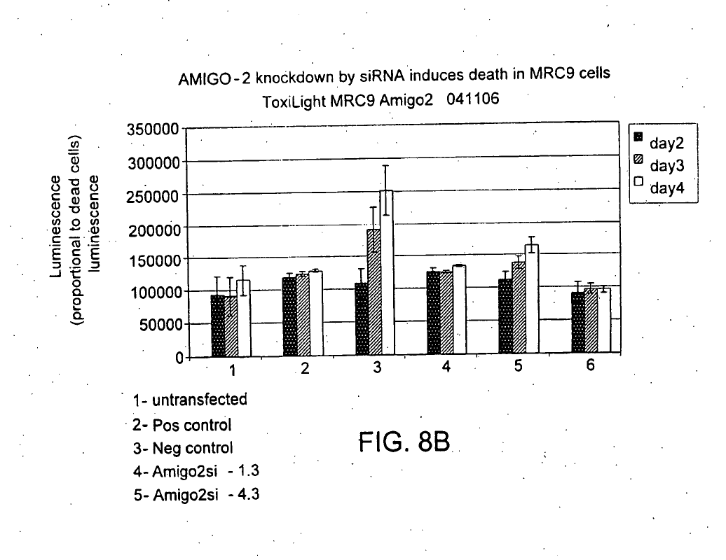

The functional consequence of AMIGO-2 knockdown was tested by several methods. A commercially available kit (ToxiLight®, Cambrex Corporation, East Rutherford, N.J.) was used to assess the degree of cell death upon AMIGO-2 knockdown in the colon cancer cell line, SW620. While very little cell death was observed in an untransfected and a negative control sample, knockdown of the positive control gene and AMIGO-2 (by two different siRNA reagents) showed significant toxicity (FIG. 5A). The functional consequence of AMIGO-2 knockdown in MRC9 cells was also examined. Although a lower amount of cell death was detected in the cells treated with the negative control siRNA, a significant and reproducible amount of cell death was observed in MRC9 cells treated with the CHIR315-1.3SI siRNA (FIG. 8B).

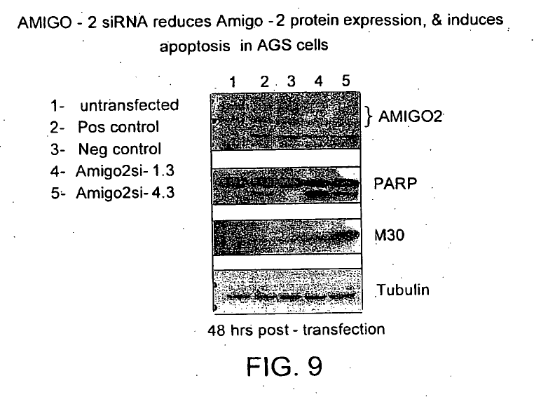

The functional consequence of AMIGO-2 knockdown was also tested by examining the effect of siRNAs CHIR315-1.3SI and CHIR315-4.3SI on PARP cleavage and M30 expression in the gastric cancer cell line, AGS. PARP cleavage and M30 production are indicative of caspase activation, which is a mediator of apoptosis. The cells were incubated with the siRNAs for 48 hrs, and then cell lysates were analyzed by Western blot for expression and processing of AMIGO-2, PARP, M30, and tubulin. PARP cleavage was increased and M30 expression was also increased in the positive control and siRNA treated samples, indicating an increase of apoptosis in these cell lines (FIG. 9). As expected, AMIGO-2 protein levels were decreased following exposure to each of the siRNAs (FIG. 9, top panel). The effect of siRNAs CHIR315-1.3SI and CHIR315-4.3SI on PARP cleavage and M30 expression was also examined in the colon cancer cell line, SW620. SW620 cells were incubated with AMIGO-2-specific siRNAs (or control siRNA) for 72 hours, and cell lysates were analyzed by Western blot as above. PARP cleavage was detected in SW620 cells treated with CHIR315-1.3SI indicating that apoptosis occurred in these cells as a result of the siRNA treatment.

Other assays were also used to examine the functional consequences of AMIGO-2 knockdown in a variety of cell lines, including AGS, SW620, HT29, A549, and Colo320 cancer cell lines; 184B5 and HMEC non-tumorigenic breast epithelial cell lines; and an MRC9 normal human lung fibroblast cell line. Experiments were performed using the two different AMIGO-2 siRNAs, C315-1.3si and C315-4.3si. AMIGO-2 knockdown was also confirmed in all experiments. The results of these experiments are as follows.

Sub-G1 DNA assays were used to measure cell death using flow cytometry. Increased cell death was detected following transfection with AMIGO-2 siRNAs in AGS, SW620, A549, and Colo320 cancer cell lines and to a small degree in HT29, but not in 184B5, or HMEC cells. No data was reported for MRC9 cells.

Apoptosis assays were also used to measure cell death by detecting PARP cleavage and/or M30 production. Increased cell death was detected following transfection with AMIGO-2 siRNAs C315-1si and C315-4si in the AGS and SW620 cancer cell lines, but not in A549 cells. No data was reported for HT29, Colo320, 184B5, HMEC, or MRC9 cell lines.

ToxiLight® assays (Cambrex Corporation, East Rutherford, N.J.) were also used to measure cell death according to manufacturer's instructions.

Cells were plated to a density that will be about 80-95% confluent after 1 day in 96-well dishes. Oligonucleotides were diluted to 2 μM in OptiMEM™. The oligonucleotide-OptiMEM™ was then added to a delivery vehicle, selected so as to be optimized for the particular cell type to be used in the assay. The oligo/delivery vehicle mixture was then further diluted into medium with serum on the cells. The final concentration of siRNA oligonucleotides was 50 nM.

Oligonucleotides were prepared as described above. Cells were transfected from about 4 hours to overnight at 37° C. and the transfection mixture was replaced with fresh medium. Transfected cells were trypsinised and counted for total cells remaining bound to plate at 48 or 72 hours.

Increased cell death was detected following transfection with AMIGO-2 siRNAs in the SW620 and A549 cancer cell lines, but not in MRC9 cells. No data was reported for AGS, HT29, Colo320, 184B5, or HMEC cell lines.

Cell titer glow (ATP measurement; Promega) assays were used to measure anchorage-dependent cell growth. At 24 hours, cells were plated on 96-well plates @ 3000-5000 cells/well. At various time-points (24 hours, 48 hours, 72 hours, 96 hours, 120 hours post-transfection), cells were lysed and assayed using cell titer glow, according to manufacturer's instructions. The output of the cell titer glow assay provides fluorescence that is proportional to relative cell number. A decrease in anchorage-dependent cell growth was observed in AGS, SW620, HT29, Colo320, A549 and MRC9 cells, but not in 184B5, or HMEC cells.

Soft-agar assays were used to measure anchorage-independent cell growth. Soft agar assays were performed by first coating a non-tissue culture treated plate with Poly-HEMA to prevent cells from attaching to the plate. Non-transfected cells were harvested using trypsin and washing twice in media. The cells were counted using a hemacytometer and resuspended to 10⁴ cells per ml in media. Fifty μl aliquots were placed in polyHEMA coated 96-well plates and transfected.

The day after transfection, cells were trypsinized, resuspended and counted. Cells were diluted to about 500 cells/100 μl/well and transferred to a deep well block (max volume=1 ml/well, in triplicate, following standard placement). Cells were plated in two plates: Corning #7007 Ultra Low Adherent U-plate for the assay, and Corning # 3799 tar the plating efficency check. Seaplaque GTG Agarose 3% was melted in a microwave oven by heating about 1 minute. When fully melted, about 10 ml was poured into pre-warmed 50 ml polypropylene tubes (Falcon # 35-2070) and incubated in the 60° C. heatblock for at least 10 minutes. About 18.6 ml complete media was added to a 50 ml polypropylene tube and incubated at about 37° C. in a water bath. A Multimek™ pipettor was used to dispense agarose to cells in 96 well plates. About 18.6 ml warm media was poured into the 10 ml of agarose and mixed well by gentle inversion. Plates were incubated at about 4° C. for 20-30 min to let the agarose solidify quickly. After agarose was solidified, 100 μl complete media was added over the cells. To measure the Day 0 plating efficiency, about 25 μl/well Alamar Blue was added and incubated overnight at 37° C. Plates were then read after 18-24 hr @ 530 ex/590 em on a TECAN plate reader. Assay plates were incubated at 37° C. for 7 days before developing with Alamar Blue (25 μl/well).

A decrease in anchorage-independent cell growth was observed in SW620, A549 and MRC9 cells. No data was reported for AGS, IIT29, or Colo320 cells. This assay is not appropriate for testing MRC9, 184B5, and HMEC cells because normal cells of this type do not typically grow in an anchorage-independent manner. Inhibition of colony formation in cancer cell lines using AMIGO-2 modulators indicates that AMIGO-2 is important in production and/or maintenance of the metastatic phenotype.

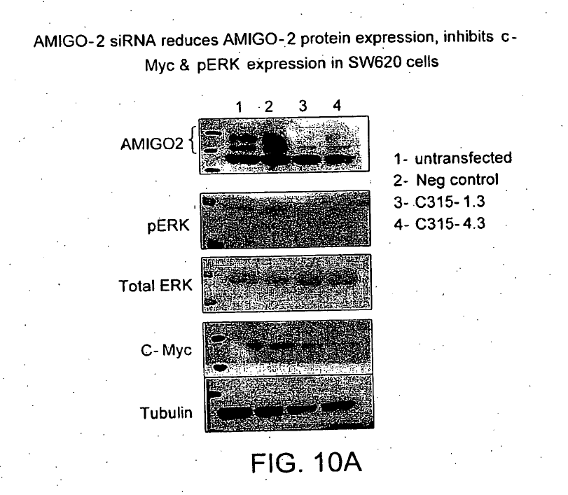

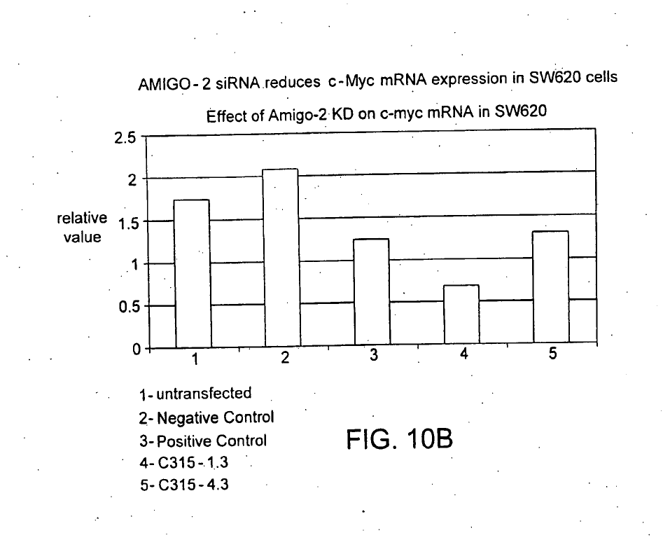

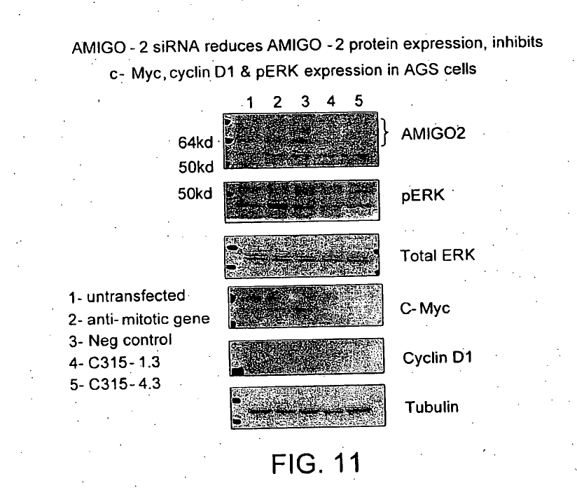

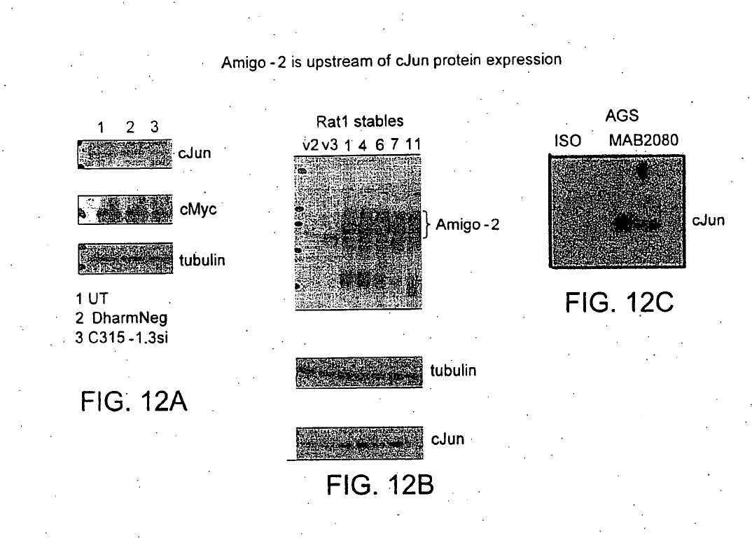

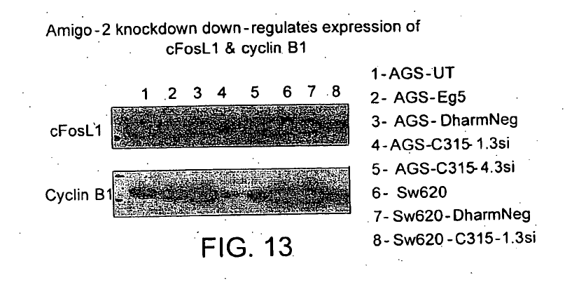

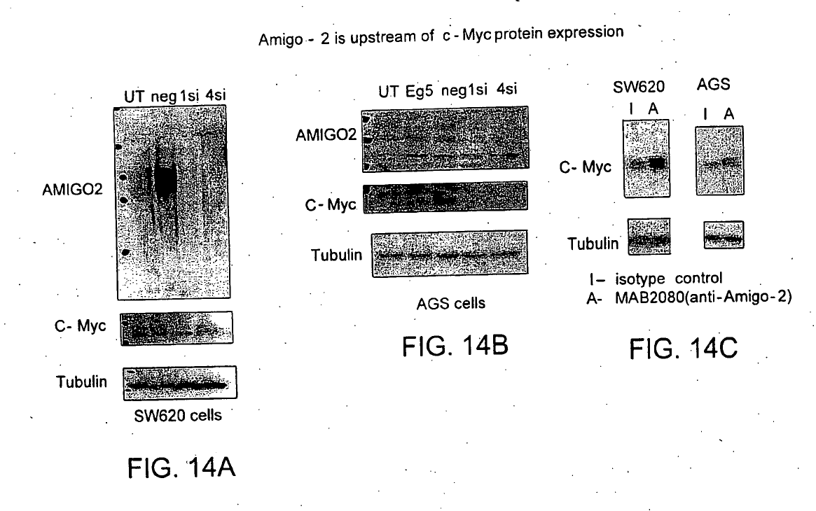

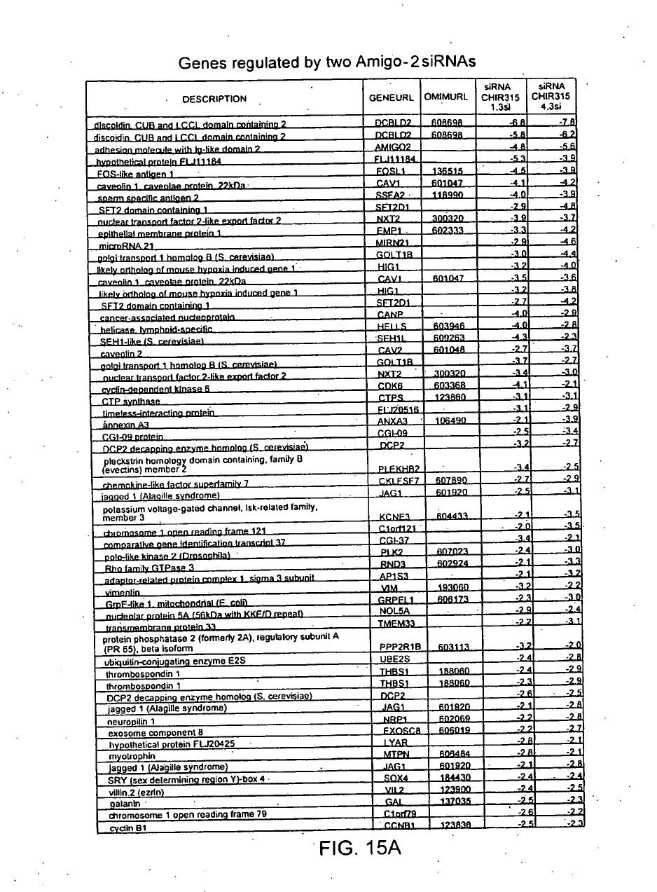

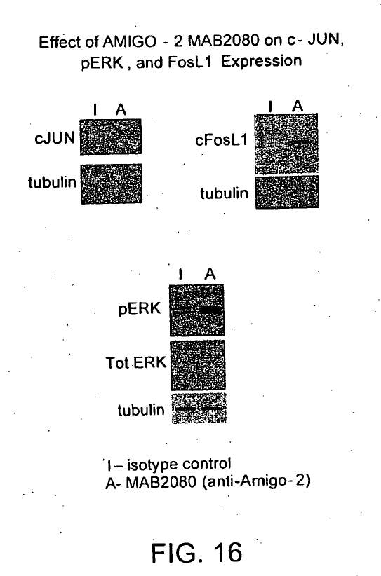

The effect of AMIGO-2 knockdown by C315-1.3si and C315-4.3si siRNAs on functionally relevant downstream markers was tested in SW620 and AGS cells. The cells were incubated with the siRNAs for 48 hrs, and then cell lysates were analyzed by Western blot. The anti-AMIGO-2 antibody RBAd70 (Chiron, Emeryville, Calif.) was used to detect AMIGO-2 protein. Tubulin was used as a loading control. Both siRNAs caused a decrease in AMIGO-2 protein levels in SW620 and AGS cells (FIGS. 10A and 11). In SW620 cells, C315-1.3si appeared to reduce AMIGO-2 protein levels more efficiently than C315-4.3si (FIG. 10A). The effect of AMIGO-2 knockdown on c-myc mRNA levels in SW620 cells was also examined using C315-1.3si and C315-4.3si siRNAs. Cells were incubated with the siRNAs for 72 hours, and then mRNA was isolated and prepared for analysis, cMyc mRNA was reduced by the positive control siRNA and both AMIGO-2-specific siRNAs, indicating that AMIGO-2 affects c-myc expression at the level of transcription (FIG. 10B). Phosphorylated ERK was also reduced in both cell lines by both siRNAs, but in SW620 cells C315-1.3si appeared to cause a greater reduction in phosphorylation than C315-4.3si (FIGS. 10 and 11, respectively). C-myc, cFosL1, and cJun were also reduced by both siRNAs in both cell lines (see FIGS. 11, 12A, 14A and 14B). Cyclin D1 expression was also decreased in AGS cells transfected with both AMIGO-2-specific siRNAs (FIG. 11). Cyclin B1 and cFosl1 levels were decreased in SW620 and AGS cells transfected with C315-1.3si (FIG. 13). SW620 cells exposed to the anti-AMIGO-2 antibody MAB2080, which acts as an agonist of AMIGO-2, exhibited upregulation of cMyc (FIG. 14C), cJun (FIG. 12C), FosL1, and phosho-ERK as determined by Western blot (FIG. 16). AGS cells contacted with MAB2080 also exhibited upregulation of cMyc (FIG. 14C) and cJun (FIG. 12C). In addition, Rat1 cells stably transfected with AMIGO-2 exhibited upregulation of cJun expression (FIG. 12B). The apparent role of AMIGO-2 in c-Myc, cyclin B1, cFosl1 and cJun expression suggests that AMIGO-2 is involved in regulation of the immediate early genes. These observations and the effect of AMIGO-2 downregulation on cyclin gene expression (see above) support a role for AMIGO-2 in cell-cycle regulation.

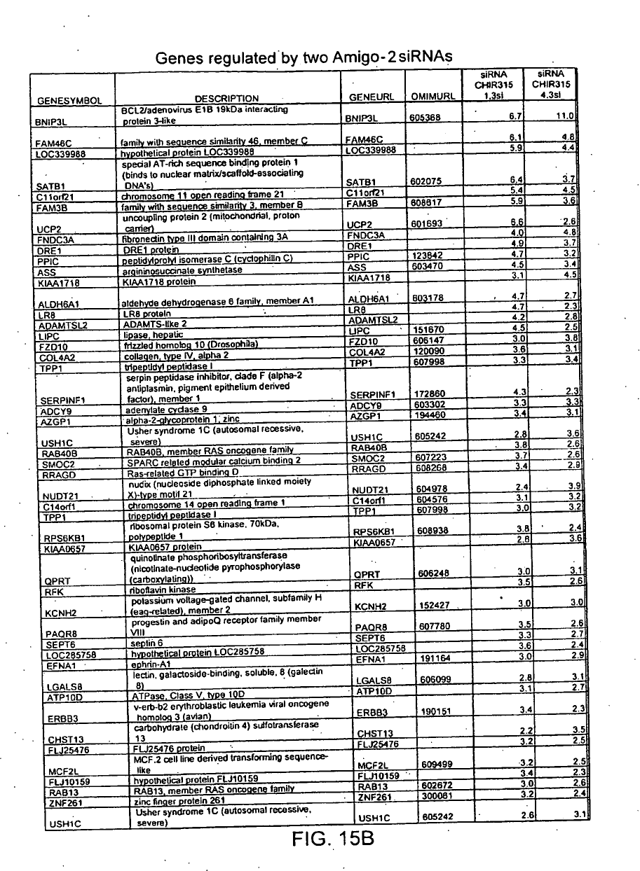

Affymetrix experiments were performed to examine the effect of AMIGO-2 siRNAs on gene expression (FIGS. 15A and 15B). These results confirmed the downregulation of cFosL1 and cyclin B1 associated with AMIGO-2 downregulation, which is consistent with a role for AMIGO-2 in cell growth and survival.

### Example 5

### AMIGO-2 Inhibits Angiogenesis

AMIGO-2 knockdown inhibits urokinase and VEGF expression, which are both involved in angiogenesis. AMIGO-2 expression has been observed in normal vessels, which supports a role for this gene in angiogenesis (see Example 2 above). Also, many genes involved in neuronal guidance and motility, like AMIGO-2, are also involved in angiogenesis.

### Example 6

### AMIGO-2 Antibodies

TABLE 1
Antibodies targeting AMIGO-2 polypeptide

| Vendor | Catalog # | Specificity | Epitope | Species |
|---|---|---|---|---|
| Chiron | RbA | Human | ECD | Rb |
| Chiron | RbB | Human | ECD | Rb |
| Abcam | ab16762 | 81% Human | TGDASADDRKAGKRVV (SEQ ID NO: 5) | Rb |
| R&D | MAB2080 | Human | Recombinant ECD (SEQ ID NO: 3) | Mouse |
| Systems | | | | |
| R&D | MAB2374 | Human | Recombinant ECD (SEQ ID NO: 3) | Rat |
| Systems | | | | |
| R&D | AF2080 | Human | Recombinant ECD (SEQ ID NO: 3) | Goat |
| Systems | | | | |
| Chiron | C3606 | Human | CIAMNKQRLLNETVDVTI (SEQ ID NO: 6) | Rb |
| Chiron | C3607 | Human | CIAMNKQRLLNETVDVTI (SEQ ID NO: 6) | Rb |

### Example 7

### AMIGO-2 Antisense Oligonucleotides

TABLE 2
Antisense RNAs targeting AMIGO-2

| | | | SEQ ID |
|---|---|---|---|
| Sample Name | Native Name | Sequence | NO: |
| CHIR315-1 | NM_181847:P0314 | GTCTGTCCGTGT | 7 |
| | | CTGTCACTCTTG | |
| CHIR315-2 | NM_181847:P0750 | ATGACGAAGAATT | 8 |
| | | AGGGTGTTCAGC | |
| CHIR315-3 | NM_181847:P0857 | TGGAATACAGCAT | 9 |
| | | TTTTCACCGTCT | |
| CHIR315-4 | NM_181847:P0900 | GTGATTGTTGTAA | 10 |
| | | AGCAGAAGCACT | |
| CHIR315-5 | NM_181847:P1160 | AAGGAGTACAGGG | 11 |
| | | AACAGTCACAGA | |
| CHIR315-6 | NM_181847:P1507 | AACGAGGGCTTTC | 12 |
| | | TATAACCAGACT | |
| CHIR315-7 | NM_181847:P1571 | ACAGTTTCATTTA | 13 |
| | | ACAGGCGTTGCT | |
| CHIR315-8 | NM_181847:P1817 | GTTCATCAGCGG | 14 |
| | | AGGCATCACTAG | |
| CHIR315-9 | NM_181847:P2526 | GGTCTAAAACGGT | 15 |
| | | AATTTTGGCTGA | |
| CHIR315-10 | NM_181847:P3219 | AGGTTCAGAGAGG | 16 |
| | | GTACTTGATTGC | |

### Example 8

### AMIGO-2 siRNA

TABLE 3
siRNAs targeting AMIGO-2 mRNA (sense strand)

| | | | SEQ ID |
|---|---|---|---|
| Sample Name | Native Name | Sequence | NO: |
| CHIR315-1SI | Qiagen | UAG AUG UUU CUU AUA ACC GAA | 17 |
| CHIR315-2SI | Qiagen | AAC UGU GGA CGU CAC AAU AAA | 18 |
| CHIR315-1.2SI | | UAG CAU CAU CAC AGA CCU AUA | 19 |
| CHIR315-1.3SI | Dharmacon-1 | GAA UAA GCA ACG CCU GUU AUU | 20 |
| | D-018701-01 (pre-val) | | |
| CHIR315-2.3SI | Dharmacon-2 | GCU CAG UGA UGG AUU UUA AUU | 21 |
| | D-018701-02 (pre-val) | | |
| CHIR315-3.2SI | | AAC UGU GGA CGU CAC AAU AAA | 22 |
| CHIR315-3.3SI | Dharmacon-3 | CCG AUG GAU UUG UAU GUU GUU | 23 |
| | D-018701-03 (pre-val) | | |
| CHIR315-4.3SI | Dharmcon-4 | GCA CUC GCG UCA GGU ACU UUU | 24 |
| | D-018701-04 (pre-val) | | |

### Example 9

### AMIGO-2 Epitopes

| | Amino Acids | | SEQ ID |
|---|---|---|---|
| Epitope | of AMIGO-2 | Domain | NO: |
| LTQFPMDLY | 177-185 | LRR5 | 25 |
| LLYNNHISY | 147-155 | LRR4 | 26 |
| GLSQLQKLY | 163-171 | LRR5 | 27 |
| YLHGNPFVC | 224-232 | LRR-CT | 28 |
| CLDLSSNKL | 121-129 | LRR3 | 29 |
| LIKRLDLSY | 69-77 | LRR1 | 30 |
| ELMFLDVSY | 193-201 | LRR6 | 31 |
| LILRHNNIT | 98-106 | LRR2 | 32 |
| LAELMFLDV | 191-199 | LRR6 | 33 |
| YSLLVFWYR | 237-245 | LRR-CT | 34 |
| LKVLEVLLL | 140-148 | LRR4 | 35 |
| YLSGNFLTQ | 171-179 | LRR5 | 36 |
| LYSLLVFWY | 236-244 | LRR-CT | 37 |
| LLQDSFMNC | 274-282 | LRR-CT | 38 |
| FSTTPNLKC | 113-121 | LRR2-LRR3 | 39 |
| DLSSNKLKTV | 123-132 | LRR3 | 40 |
| KLAELMFLDV | 190-199 | LRR6 | 41 |
| CLLMITVTV | 25-33 | Signal peptide | 42 |
| LLCLLMITV | 23-31 | Signal peptide | 43 |
| IYLHGNPFV | 223-231 | LRR-CT | 44 |
| KVLEVLLLY | 141-149 | LRR4 | 45 |
| RIPSMPMHH | 204-212 | LRR6 | 46 |
| NLVPGKQLR | 213-221 | LRR6 | 47 |
| GAVVRPGCR | 13-21 | Signal peptide | 48 |
| RHSRQVLLL | 267-275 | LRR-CT | 49 |
| LRGIYLHGN | 220-228 | LRR-CT | 50 |
| YRRHFSSVM | 244-252 | LRR-CT | 51 |
| RRHFSSVMDF | 245-254 | LRR-CT | 52 |
| GRFKLAELM | 187-195 | LRR5 | 53 |
| NPFVCDCSL | 228-236 | LRR-CT | 54 |
| LPTLLGAVV | 8-16 | Signal peptide | 55 |
| TPNLKCLDL | 116-124 | LRR3 | 56 |
| RLIKRLDLSY | 68-77 | LRR1 | 57 |
| KNAVFQELK | 133-141 | LRR3 | 58 |
| SLLVFWYRR | 238-246 | LRR-CT | 59 |
| RRHFSSVMD | 245-253 | LRR-CT | 60 |
| LRHNNITSI | 100-108 | LRR2 | 61 |
| VPGKQLRGI | 215-223 | LRR6-LRRCT | 62 |

### Example 10

### AMIGO-2 Expression in Normal Tissues

Normal tissue sections were prepared and stained with rabbit anti-AMIGO-2 or control, prebleed antibodies as described above. Staining indicated that AMIGO-2 is expressed in a variety normal human tissues, including adrenal, breast, cervix, lung, kidney, liver, ovary, pancreas, prostate, skeletal muscle, skin, spleen, testes, colon, and uterus, particularly in stromal cell subsets (e.g., vessels and macrophages) of lung, cervix, heart, and liver. AMIGO-2 protein expression levels in these tissues correlated with corresponding mRNA expression levels determined by Affymetrix experimentation (see above), and indicated that AMIGO-2 is widely expressed in normal tissues, particularly in proliferative tissues (e.g., testes, skin, ovary, spleen, and colon).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention.

What is claimed is:

1. A method of treating cancer or a cancer symptom in a patient in need thereof comprising administering to the patient a therapeutically effective amount of an AMIGO-2 inhibitor, said AMIGO-2 inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

2. The method of claim 1, wherein the AMIGO-2 inhibitor inhibits AMIGO-2 expression by at least 20% as compared to a control.

3. The method of claim 1, wherein the AMIGO-2 inhibitor causes cell death in at least 20% of cancer cells as compared to a control.

4. The method of claim 1, wherein the AMIGO-2 inhibitor is a monoclonal antibody, a polyclonal antibody, a chimeric antibody, a human antibody, a humanized antibody, a single-chain antibody, or a Fab fragment.

5. The method of claim 87, wherein the antibody or Fab fragment binds to one or more epitopes in the ECD of AMIGO-2.

6. The method of claim 1, wherein the antibody or Fab fragment specifically binds to one or more epitopes in a sequence consisting essentially of SEQ ID NO:2 or SEQ ID NO:3.

7. The method of claim 1, wherein the antibody or Fab fragment specifically binds to one or more epitopes of the LRRNT domain of AMIGO-2.

8. The method of claim 1, wherein the antibody or Fab fragment specifically binds to one or more epitopes of the LRR1 domain of AMIGO-2, said epitope selected from the group consisting of SEQ ID NO:30 and SEQ ID NO:57.

9. The method of claim 1, wherein the antibody or Fab fragment specifically binds to one or more epitopes of the LRR2 domain of AMIGO-2, said epitope selected from the group consisting of SEQ ID NO:32, SEQ ID NO:39, and SEQ ID NO:61.

10. The method of claim 1, wherein the antibody or Fab fragment specifically binds to one or more epitopes of the LRR3 domain of AMIGO-2, said epitope selected from the group consisting of SEQ ID NO:29, SEQ ID NO:39, SEQ ID NO:40, SEQ ID NO:56, and SEQ ID NO:58.

11. The method of claim 1, wherein the antibody or Fab fragment specifically binds to one or more epitopes of the LRR4 domain of AMIGO-2, said epitope selected from the group consisting of SEQ ID NO:26, SEQ ID NO:35, and SEQ ID NO:45.

12. The method of claim 1, wherein the antibody or Fab fragment specifically binds to one or more epitopes of the LRR5 domain of AMIGO-2, said epitope selected from the group consisting of SEQ ID NO:25, SEQ ID NO:27, SEQ ID NO:36, and SEQ ID NO:53.

13. The method of claim 1, wherein the antibody or Fab fragment specifically binds to one or more epitopes of the LRR6 domain of AMIGO-2, said epitope selected from the group consisting of SEQ ID NO:31, SEQ ID NO:33, SEQ ID NO:41, SEQ ID NO:46, SEQ ID NO:47, and SEQ ID NO:62.

14. The method of claim 1, wherein the antibody or Fab fragment specifically binds to one or more epitopes of the LRRCT domain of AMIGO-2, said epitope selected from the group consisting of SEQ ID NO:28, SEQ ID NO:34, SEQ ID NO:37, SEQ ID NO:38, SEQ ID NO:44, SEQ ID NO:49, SEQ ID NO:50, SEQ ID NO:51, SEQ ID NO:52, SEQ ID NO:54, SEQ ID NO:59, SEQ ID NO:60, and SEQ ID NO:62.

15. The method of claim 1, wherein the antibody or Fab fragment specifically binds to one or more epitopes of the Ig V-set domain of AMIGO-2.

16. The method of claim 1, wherein the antibody or Fab fragment specifically binds to one or more epitopes of the Ig domain of AMIGO-2.

17. The method of claim 1, wherein the antibody or Fab fragment specifically binds one or more epitopes in the ECD of AMIGO-2.

18. The method of claim 1, wherein the antibody or Fab fragment specifically binds to one or more epitopes selected from the group consisting of SEQ ID NOs:3-6 and 25-62.

19. The method of claim 1, wherein the patient has or is predisposed to lung, bladder, kidney, colon, breast, uterine, ovarian, or pancreatic cancer.

20. The method of claim 1, wherein the cancer is lung or colon cancer.

21. The method of claim 4, wherein the antibody or Fab fragment inhibits an AMIGO-2 activity selected from the group consisting of chromosomal stability, tumorigenicity, cell proliferation, cell cycle regulation, cancer cell motility, cell adhesion, tumor formation, metastasis, AMIGO-2 signaling, cancer cell survival, cyclin production, kinase activity, substrate phosphorylation, anchorage-independent growth, localization of AMIGO-2 protein to the cell-membrane, interactions between AMIGO-2 and one or both of AMIGO-1 or AMIGO-3, levels of cytoplasmic phosphorylated AMIGO-2 protein, interactions between tumor and stromal tissue, and angiogenesis.

22. The method of claim 4, wherein the antibody is labeled.

23. The method of claim 22, wherein the label is an enzyme, radioisotope, toxin or fluorophore.

24. The method of claim 4, wherein the antibody has a binding affinity less than about 1×10⁵Kₐ for a polypeptide other than AMIGO-2.

25. The method of claim 1, wherein the AMIGO-2 inhibitor is a dsRNA molecule comprising a first strand of nucleotides comprising at least 19 consecutive nucleotides of a sequence set forth in SEQ ID NOs:17-24, and a second strand of nucleotides comprising a sequence substantially complementary to the first strand, wherein the dsRNA molecule is less than 3769 nucleotides long.

26. The method of claim 1, wherein the AMIGO-2 inhibitor is an isolated nucleic acid comprising at least 10 consecutive nucleotides of a sequence set forth in SEQ ID NOs:7-16.

27. The method of claim 1, further comprising the treatment of the patient with one or more of chemotherapy, radiation therapy or surgery.

28. The method of claim 1, wherein the cancer symptom is selected from the group consisting of a chronic cough, worsening breathlessness, weight loss, excessive fatigue, pain, coughing up blood, blood in the urine, loss of appetite, heavy sweating, fever, high blood pressure, anemia, diarrhea, constipation, blood in the stool, jaundice, dizziness, weakness, chills, muscle spasms, deep vein thrombosis, abdominal distension, bloating, irregular menses, colon metastases, lung metastases, bladder metastases, kidney metastases, breast metastases, uterine metastases, ovarian metastases, and pancreas metastases.

29. A method of modulating an AMIGO-2 activity in a patient, the method comprising administering to the patient an amount of an AMIGO-2 inhibitor effective to modulate the AMIGO-2 activity, said AMIGO-2 inhibitor selected from the group consisting of:
(a) an antibody that specifically binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

30. The method of claim 29, wherein the AMIGO-2 activity is selected from the group consisting of chromosomal stability, tumorigenicity, cell proliferation, cell cycle regulation, cancer cell motility, cell adhesion, tumor formation, metastasis, AMIGO-2 signaling, modulation of a downstream marker of AMIGO-2, cancer cell survival, cyclin production, kinase activity, substrate phosphorylation, anchorage-independent growth, localization of AMIGO-2 protein to the cell-membrane, interactions between AMIGO-2 and one or both of AMIGO-1 or AMIGO-3, levels of cytoplasmic phosphorylated AMIGO-2 protein, interactions between tumor and stromal tissue, and angiogenesis.

31. The method of claim 29, wherein the AMIGO-2 inhibitor is a monoclonal antibody, a polyclonal antibody, a chimeric antibody, a human antibody, a humanized antibody, a single-chain antibody, or a Fab fragment.

32. A method of identifying a patient susceptible to AMIGO-2 therapy comprising:
(a) detecting the presence or absence of evidence of AMIGO-2 expression in said sample, wherein the presence of evidence of AMIGO-2 expression in said sample is indicative of a patient who is a candidate for AMIGO-2 therapy and the absence of evidence of AMIGO-2 expression in said sample is indicative of a patient who is not a candidate for AMIGO-2 therapy;
(b) administering a therapeutically effective amount of an inhibitor selected from the group consisting of:
(1) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(2) an isolated oligonucleotide comprising at least 10 consecutive nucleotides of a sequence selected from the group consisting of SEQ ID NOs:7-16;
(3) an isolated double-stranded RNA (dsRNA);
(4) a small molecule;
(5) a mimetic;
(6) a soluble receptor; and
(7) a decoy
to the patient if the patient is a candidate for AMIGO-2 therapy; and
(c) administering a traditional cancer therapeutic to the patient if the patient is not a candidate for AMIGO-2 therapy.

33. The method of claim 32, wherein the expression of AMIGO-2 is increased at least 20% compared to a control.

34. The method of claim 32, wherein evidence of AMIGO-2 expression is detected by measuring AMIGO-2 RNA levels.

35. The method of claim 32, wherein evidence of AMIGO-2 expression is detected by measuring AMIGO-2 polypeptide levels.

36. The method of claim 32, wherein the patient has or is predisposed to one or more of lung, bladder, kidney, colon, breast, uterine, ovarian, or pancreatic cancer.

37. A method of inhibiting growth of cancer cells comprising contacting the cancer cells with an amount of an AMIGO-2 inhibitor effective to inhibit growth of the cells by at least 20% as compared to a control, said inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

38. The method of claim 37 wherein the cancer cells are in or are derived from a cancer patient.

39. The method of claim 37, wherein the AMIGO-2 inhibitor is a monoclonal antibody, a polyclonal antibody, a chimeric antibody, a human antibody, a humanized antibody, a single-chain antibody, or a Fab fragment.

40. A method of inhibiting a cancer cell phenotype in a patient in need thereof, said method comprising administering to said patient a therapeutically effective amount of an AMIGO-2 inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

41. The method of claim 40, wherein the cancer cell phenotype is one or more of cell motility in collagen, tumorigenicity, ability to grow in an anchorage-independent manner, cell survival, or cell adhesion.

42. The method of claim 40, wherein the cancer cells are selected from the group consisting of lung, bladder, kidney, colon, breast, uterine, ovarian, and pancreatic cancer cells.

43. A method of inhibiting cancer cell growth, the method comprising administering to a patient having a cancer comprising one or more cells expressing AMIGO-2 a compound that modulates of one or more downstream markers of AMIGO-2.

44. The method of claim 43, wherein the one or more downstream markers of AMIGO-2 are selected from the group consisting of c-MYC, c-Jun, FosL1, and Extracellular signal-Regulated Kinase (ERK).

45. The method of claim 44, wherein the ERK is phosphorylated ERK.

46. A method for detecting a tumor in a patient comprising administering to the patient a composition comprising an AMIGO-2 inhibitor linked to an imaging agent and detecting the localization of the imaging agent in the patient, wherein said inhibitor is selected from the group consisting of
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

47. The method of claim 46, wherein the composition comprises an AMIGO-2 antibody conjugated to an imaging agent.

48. The method of claim 47, wherein the imaging agent is ¹⁸F, ⁴³K, ⁵²Fe, ⁵⁷Co, ⁶⁷Cu, ⁶⁷Ga, ⁷⁷Br, ⁸⁷MSr, ⁸⁶Y, ⁹⁰Y, ⁹⁹MTc, ¹¹¹In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi.

49. A method of identifying a cancer inhibitor, the cancer characterized by overexpression of AMIGO-2 compared to a control, said method comprising contacting a cell expressing AMIGO-2 with a candidate compound and determining whether an AMIGO-2 activity is modulated, wherein modulation of the AMIGO-2 activity is indicative of a cancer inhibitor.

50. The method of claim 49, wherein the candidate compound modulates the AMIGO-2 activity in cancer cells, but not in non-cancer cells.

51. A method of identifying a cancer inhibitor, said cancer characterized by overexpression of AMIGO-2 compared to a control, said method comprising contacting a cell expressing AMIGO-2 with a candidate compound and an AMIGO-2 ligand, and determining whether an activity of a downstream marker of AMIGO-2 is modulated, wherein modulation of the downstream marker is indicative of a cancer inhibitor.

52. The method of claim 51, wherein the downstream marker is selected from the group consisting of decreased expression of cyclin D1, cyclin B1, c-Myc, c-Jun, FosL1, Extracellular signal-Regulated Kinase (ERK), Vascular Endothelial Growth Factor (VEGF), urokinase, and Poly(ADP-Ribose)Polymerase 1 (PARP1).

53. The method of claim 51, wherein the activity of the downstream marker is decreased ERK phosphorylation or decreased ERK expression.

54. The method of claim 51, wherein the activity of the downstream marker is increased PARP1 cleavage.

55. The method of claim 51, wherein the candidate compound and AMIGO-2 ligand induce the modulation of a downstream marker of AMIGO-2 in cancer cells, but not in non-cancer cells.

56. A method of delivering a cytotoxic agent or a diagnostic agent to one or more cells that express AMIGO-2, said method comprising:
(a) providing the cytotoxic agent or the diagnostic agent conjugated to a purified antibody that specifically binds to an epitope of an AMIGO-2 polypeptide, wherein the epitope is in the domain selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain; and
(b) exposing the cell to the antibody-agent or fragment-agent conjugate.

57. A method of treating a cancer patient comprising comparing AMIGO-2 expression in a cancer sample from the patient to AMIGO-2 expression in a control sample and:
(1) treating the patient with a composition comprising an inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy,
if AMIGO-2 expression is upregulated in the cancer sample as compared to the control sample; and
(2) performing a secondary assay if AMIGO-2 expression is unchanged or downregulated in the cancer sample as compared to the control sample.

58. The method of claim 57, wherein the secondary assay comprises comparing a level or activity of an AMIGO-2 downstream marker in the cancer sample in the presence and absence of an inhibitor, wherein:
(1) the patient is treated with a composition comprising an inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy,
if the level or activity of the AMIGO-2 downstream marker in the cancer sample is decreased in the presence of an AMIGO-2 inhibitor compared to the level or activity of the AMIGO-2 downstream marker in the cancer sample in the absence of the AMIGO-2 inhibitor; or
(2) the patient is treated with a traditional cancer therapeutic if the level or activity of the AMIGO-2 downstream marker in the cancer sample is unchanged or decreased in the presence of an AMIGO-2 inhibitor compared to the level or activity of the AMIGO-2 downstream marker in the cancer sample in the absence of the AMIGO-2 inhibitor.

59. The method of claim 57, wherein the AMIGO-2 downstream marker is selected from the group consisting of cyclin D1, cyclin B1, c-Myc, c-Jun, FosL1, VEGF, urokinase, and ERK.

60. The method of claim 57, wherein the activity of the downstream marker is ERK phosphorylation or ERK expression.

61. The method of claim 57, wherein the activity of the downstream AMIGO-2 marker is decreased.

62. The method of claim 57 wherein the secondary assay comprises comparing PARP1 cleavage in the cancer sample in the presence and absence of an inhibitor, wherein:
(a) the patient is treated with a composition comprising an inhibitor selected from the group consisting of:
(1) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(2) an isolated oligonucleotide comprising at least 10 consecutive nucleotides of a sequence selected from the group consisting of SEQ ID NOs:7-16;
(3) an isolated double-stranded RNA (dsRNA);
(4) a small molecule;
(5) a mimetic;
(6) a soluble receptor; and
(7) a decoy,
if PARP1 cleavage is increased in the cancer sample in the presence of an AMIGO-2 inhibitor compared to PARP1 cleavage in the cancer sample in the absence of the AMIGO-2 inhibitor; or
(b) the patient is treated with a traditional cancer therapeutic if PARP1 cleavage is increased or unchanged in the cancer sample in the presence of an AMIGO-2 inhibitor compared to PARP1 cleavage in the cancer sample in the absence of the AMIGO-2 inhibitor.

63. A method for diagnosing cancer in a patient comprising assaying for AMIGO-2 localization in candidate cancer cells, wherein when the ratio of AMIGO-2 localized to the cell membrane to AMIGO-2 localized to other areas of the cancer cells not including the cell membrane is at least 2:1, the patient is diagnosed as having an AMIGO-2-related cancer.

64. The method of claim 63, wherein the ratio of AMIGO-2 localized to the cell membrane to AMIGO-2 localized to other areas of the cancer cells not including the cell membrane is at least 3:1.

65. The method of claim 63, further comprising administering a composition comprising an inhibitor selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy,
to the patient when the patient is diagnosed as having an AMIGO-2-related cancer.

66. A method of identifying an AMIGO-2 modulator comprising comparing phosphorylation of AMIGO-2 in a sample comprising one or more cells expressing AMIGO-2 in the presence and absence of a candidate compound, wherein modulation of phosphorylation of AMIGO-2 in the sample in the presence of the candidate compound as compared to phosphorylation of AMIGO-2 in the sample in the absence of the candidate compound indicates that the candidate compound is an AMIGO-2 modulator.

67. The method of claim 66, wherein AMIGO-2 is isolated from the sample using an immunoprecipitating antibody.

68. The method of claim 67, wherein the immunoprecipitating antibody is an antibody that specifically binds to an epitope of an AMIGO-2 polypeptide, wherein the epitope is in the domain selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain.

69. The method of claim 66, wherein the AMIGO-2 phosphorylation is serine/threonine phosphorylation.

70. The method of claim 66, wherein the AMIGO-2 phosphorylation is determined using a phosphoserine/threonine antibody.

71. The method of claim 67, wherein the immunoprecipitating antibody is a phosphoserine/threonine antibody.

72. A composition comprising an AMIGO-2 inhibitor and one or more pharmaceutically acceptable carriers, wherein the AMIGO-2 inhibitor is selected from the group consisting of:
(a) an antibody that binds an epitope in a domain of AMIGO-2 selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain;
(b) an isolated oligonucleotide comprising at least 10 consecutive nucleotides of a sequence selected from the group consisting of SEQ ID NOs:7-16;
(c) an isolated double-stranded RNA (dsRNA);
(d) a small molecule;
(e) a mimetic;
(f) a soluble receptor; and
(g) a decoy.

73. The composition of claim 72, wherein the composition inhibits at least one AMIGO-2 activity selected from the group consisting of chromosomal stability, tumorigenicity, cell proliferation, cell cycle regulation, cancer cell motility, cell adhesion, tumor formation, metastasis, AMIGO-2 signaling, cancer cell survival, cyclin production, kinase activity, substrate phosphorylation, anchorage-independent growth, localization of AMIGO-2 protein to the cell-membrane, interactions between AMIGO-2 and one or both of AMIGO-1 or AMIGO-3, levels of cytoplasmic phosphorylated AMIGO-2 protein, interactions between tumor and stromal tissue and angiogenesis.

74. The composition of claim 72, wherein the composition induces at least one cell phenotype in cancer cells, but not in non-cancer cells.

75. The composition of claim 72, wherein the composition inhibits cancer cell survival, composition inhibits cytoplasmic phosphorylation of AMIGO-2, inhibits angiogenesis or cell proliferation, serine/threonine kinase activity, inhibits Erk phosphorylation, inhibits cJun, cMyc, or FosL1 expression, or inhibits progression of dividing cells into the G2/M stage of the cell cycle.

76. The composition of claim 72, wherein the composition is a sterile injectable.

77. The composition of claim 72, wherein the AMIGO-2 inhibitor inhibits AMIGO-2 translation or induces AMIGO-2 mRNA degradation.

78. The composition of claim 72, wherein the AMIGO-2 inhibitor is a monoclonal antibody, a polyclonal antibody, a chimeric antibody, a human antibody, a humanized antibody, a single-chain antibody, or a Fab fragment.

79. The composition of claim 78, wherein the antibody or Fab fragment specifically binds to one or more epitopes of the LRRNT domain of AMIGO-2.

80. The composition of claim 78, wherein the antibody or Fab fragment specifically binds to one or more epitopes of the LRR1 domain of AMIGO-2, said epitope selected from the group consisting of SEQ ID NO:30 and SEQ ID NO:57.

81. The composition of claim 78, wherein the antibody or Fab fragment specifically binds to one or more epitopes of the LRR2 domain of AMIGO-2, said epitope selected from the group consisting of SEQ ID NO:32, SEQ ID NO:39, and SEQ ID NO:61.

82. The composition of claim 78, wherein the antibody or Fab fragment specifically binds to one or more epitopes of the LRR3 domain of AMIGO-2, said epitope selected from the group consisting of SEQ ID NO:29, SEQ ID NO:39, SEQ ID NO:40, SEQ ID NO:56, and SEQ ID NO:58.

83. The composition of claim 78, wherein the antibody or Fab fragment specifically binds to one or more epitopes of the LRR4 domain of AMIGO-2, said epitope selected from the group consisting of SEQ ID NO:26, SEQ ID NO:35, and SEQ ID NO:45.

84. The composition of claim 78, wherein the antibody or Fab fragment specifically binds to one or more epitopes of the LRR5 domain of AMIGO-2, said epitope selected from the group consisting of SEQ ID NO:25, SEQ ID NO:27, SEQ ID NO:36, and SEQ ID NO:53.

85. The composition of claim 78, wherein the antibody or Fab fragment specifically binds to one or more epitopes of the LRR6 domain of AMIGO-2, said epitope selected from the group consisting of SEQ ID NO:31, SEQ ID NO:33, SEQ ID NO:41, SEQ ID NO:46, SEQ ID NO:47, and SEQ ID NO:62.

86. The composition of claim 78, wherein the antibody or Fab fragment specifically binds to one or more epitopes of the LRR-CT domain of AMIGO-2, said epitope selected from the group consisting of SEQ ID NO:28, SEQ ID NO:34, SEQ ID NO:37, SEQ ID NO:38, SEQ ID NO:44, SEQ ID NO:49, SEQ ID NO:50, SEQ ID NO:51, SEQ ID NO:52, SEQ ID NO:54, SEQ ID NO:59, SEQ ID NO:60, and SEQ ID NO:62.

87. The composition of claim 78, wherein the antibody or Fab fragment specifically binds to one or more epitopes of the Ig V-set domain of AMIGO-2.

88. The composition of claim 78, wherein the antibody or Fab fragment specifically binds to one or more epitopes of the Ig domain of AMIGO-2.

89. The composition of claim 78, wherein the antibody or Fab fragment specifically binds one or more epitopes in the extracellular domain (ECD) of AMIGO-2.

90. The composition of claim 78, wherein the antibody or Fab fragment specifically binds to one or more epitopes in a sequence consisting essentially of a sequence selected from the group consisting of SEQ ID NOs:3-6 and 25-62.

91. The composition of claim 78, wherein the antibody or Fab fragment specifically binds to one or more epitopes in the sequence consisting essentially of SEQ ID NO:2 or SEQ ID NO:3.

92. The composition of claim 78, wherein the antibody or Fab fragment binds to AMIGO-2 with an affinity of at least 1×10⁸ Kₐ.

93. The composition of claim 72, wherein the AMIGO-2 activity is selected from the group consisting of chromosomal stability, tumorigenicity, cell proliferation, cell cycle regulation, cancer cell motility, cell adhesion, tumor formation, metastasis, AMIGO-2 signaling, cancer cell survival, cyclin production, kinase activity, substrate phosphorylation, anchorage-independent growth, localization of AMIGO-2 protein to the cell-membrane, interactions between AMIGO-2 and one or both of AMIGO-1 or AMIGO-3, levels of cytoplasmic phosphorylated AMIGO-2 protein, interactions between tumor and stromal tissue, and angiogenesis.

94. The composition of claim 72, wherein the antibody or Fab fragment inhibits cancer cell survival, cytoplasmic phosphorylation of AMIGO-2, or Erk phosphorylation.

95. The composition of claim 72, wherein the composition inhibits cJun, cMyc, or FosL1 expression.

96. The composition of claim 72, wherein the composition inhibits progression of dividing cells into the G2/M stage of the cell cycle.

97. The composition of claim 72, wherein the AMIGO-2 inhibitor is a dsRNA molecule comprising a first strand of nucleotides comprising at least 19 consecutive nucleotides of a sequence selected from the group consisting of SEQ ID NOs:17-24, and a second strand of nucleotides comprising a sequence substantially complementary to the first strand, wherein the dsRNA molecule is less than 3769 nucleotides long.

98. The composition of claim 97, wherein the dsRNA inhibits AMIGO-2 translation by at least 20% as compared to a control.

99. A purified antibody that specifically binds to an epitope of an AMIGO-2 polypeptide, wherein the epitope is in the domain selected from the group consisting of the signal peptide, the LRRNT domain, LRR1 domain, LRR2 domain, LRR3 domain, LRR4 domain, LRR5 domain, LRR6 domain, LRRCT domain, Ig V-set domain, and Ig domain.

100. The purified antibody of claim 99, wherein the antibody specifically binds to one or more epitopes of the LRR1 domain of AMIGO-2, said epitope selected from the group consisting of SEQ ID NO:30 and SEQ ID NO:57.

101. The purified antibody of claim 99, wherein the antibody specifically binds to one or more epitopes of the LRR2 domain of AMIGO-2, said epitope selected from the group consisting of SEQ ID NO:32, SEQ ID NO:39, and SEQ ID NO:61.

102. The purified antibody of claim 99, wherein the antibody specifically binds to one or more epitopes of the LRR3 domain of AMIGO-2, said epitope selected from the group consisting of SEQ ID NO:29, SEQ ID NO:39, SEQ ID NO:40, SEQ ID NO:56, and SEQ ID NO:58.

103. The purified antibody of claim 99, wherein the antibody specifically binds to one or more epitopes of the LRR4 domain of AMIGO-2, said epitope selected from the group consisting of SEQ ID NO:26, SEQ ID NO:35, and SEQ ID NO:45.

104. The purified antibody of claim 99, wherein the antibody specifically binds to one or more epitopes of the LRR5 domain of AMIGO-2, said epitope selected from the group consisting of SEQ ID NO:25, SEQ ID NO:27, SEQ ID NO:36, and SEQ ID NO:53.

105. The purified antibody of claim 99, wherein the antibody specifically binds to one or more epitopes of the LRR6 domain of AMIGO-2, said epitope selected from the group consisting of SEQ ID NO:31, SEQ ID NO:33, SEQ ID NO:41, SEQ ID NO:46, SEQ ID NO:47, and SEQ ID NO:62.

106. The purified antibody of claim 99, wherein the antibody specifically binds to one or more epitopes of the LRRCT domain of AMIGO-2, said epitope selected from the group consisting of SEQ ID NO:28, SEQ ID NO:34, SEQ ID NO:37, SEQ ID NO:38, SEQ ID NO:44, SEQ ID NO:49, SEQ ID NO:50, SEQ ID NO:51, SEQ ID NO:52, SEQ ID NO:54, SEQ ID NO:59, SEQ ID NO:60, and SEQ ID NO:62.

107. The purified antibody of claim 99, wherein the antibody specifically binds to one or more epitopes selected from the group consisting of SEQ ID NOs:25-62.

108. The purified antibody of claim 119, wherein the antibody specifically binds to one or more epitopes in the sequence consisting essentially of SEQ ID NO:2 or SEQ ID NO:3.

109. The purified antibody of claim 99, wherein the antibody inhibits an AMIGO-2 activity selected from the group consisting of chromosomal stability, tumorigenicity, cell proliferation, cell cycle regulation, cancer cell motility, cell adhesion, tumor formation, metastasis, AMIGO-2 signaling, cancer cell survival, cyclin production, kinase activity, substrate phosphorylation, anchorage-independent growth, localization of AMIGO-2 protein to the cell-membrane, interactions between AMIGO-2 and one or both of AMIGO-1 or AMIGO-3, levels of cytoplasmic phosphorylated AMIGO-2 protein, interactions between tumor and stromal tissue, and angiogenesis.

110. A purified antibody that specifically binds to one or more epitopes of an AMIGO-2 polypeptide, wherein the epitope comprises a sequence selected from the group consisting of SEQ ID NOs:3-6 and 25-62.

111. An isolated cell that produces the antibody of claim 99.

112. A hybridoma that produces the antibody of claim 99.

113. A non-human transgenic animal that produces the antibody of claim 99.

114. An isolated epitope-bearing fragment of the polypeptide of SEQ ID NO:2, said fragment comprising one or more epitopes selected from the group consisting of SEQ ID NOs:3-6 and 25-62.

115. The epitope-bearing fragment of claim 114, which comprises between about 6 and about 20 contiguous amino acids of SEQ ID NO:2.

116. The epitope-bearing fragment of claim 114, which comprises at least 21 contiguous amino acids of SEQ ID NO:2 and less than 522 contiguous amino acids of SEQ ID NO:2.

117. A polynucleotide that encodes an isolated epitope-bearing fragment of claim 114.

118. A purified AMIGO-2 antibody which is obtained by immunization of a subject with the epitope-bearing fragment of claim 114.

119. An isolated dsRNA molecule comprising a first strand of nucleotides comprising at least 19 consecutive nucleotides of a sequence set forth in SEQ ID NOs:17-24, and a second strand of nucleotides comprising a sequence substantially complementary to the first strand, wherein the dsRNA molecule is less than 3769 nucleotides long.

120. The isolated dsRNA molecule of claim 119, wherein the second strand of nucleotides comprises a sequence fully complementary to the first strand, wherein the dsRNA molecule is less than 3769 nucleotides long.

121. An isolated nucleic acid comprising at least 10 consecutive nucleotides of a sequence set forth in SEQ ID NOs:7-16.
